(19) United States
(12) Patent Application Publication
(10) Pub. No.: US 2025/0341454 A1
(43) Pub. Date: Nov. 6, 2025

(54) SOLID-STATE AND CONFIGURABLE OPTICAL TEST TARGETS AND FLOW CELL DEVICES

(71) Applicant: Element Biosciences, Inc., San Diego, CA (US)

(72) Inventors: Hui Zhen MAH, San Diego, CA (US); Sanchari SAHA, San Diego, CA (US); Michael PREVITE, San Diego, CA (US); Xiyi CHEN, Pearland, TX (US); Derek FULLER, San Diego, CA (US); Chiting CHANG, San Diego, CA (US); Cassandra NIMAN, San Diego, CA (US); Steve CHEN, San Diego, CA (US); Arash GHORBANI, San Diego, CA (US); Minghao GUO, San Diego, CA (US); Chaoyi JIN, San Diego, CA (US); Rui MA, San Diego, CA (US); Jordan NEYSMITH, San Diego, CA (US); Geoffrey PILAND, San Diego, CA (US); Daisong RONG, San Diego, CA (US); Bowen SCHWOERER, San Diego, CA (US); Russell HUDYMA, San Diego, CA (US)

(21) Appl. No.: 19/269,395

(22) Filed: Jul. 15, 2025

Related U.S. Application Data
(63) Continuation of application No. PCT/IB2024/059112, filed on Sep. 19, 2024.
(60) Provisional application No. 63/584,056, filed on Sep. 20, 2023.

(51) Int. Cl. G01N 15/1429; C12Q 1/6869; G01N 15/01; G01N 15/1434; G01N 21/64

(52) U.S. Cl. CPC G01N 15/1429; C12Q 1/6869; G01N 15/01; G01N 15/1436; G01N 2021/6439

(57) ABSTRACT

The present disclosure provides solid-state optical test targets useful for evaluating the performance of an optical imaging system. The solid-state optical test targets comprise at least a first substrate made from a flat transparent material. In some embodiments, the solid-state optical test targets further comprise an opaque coating that forms a micropattern. The first substrate is positioned in direct contact with the micropattern. The optical test targets described herein lack a flow cell and lack a liquid, and are therefore solid-state apparatus. Since the solid-state optical test targets lack a flow cell and liquid, the thickness of the first substrate is adjusted to simulate the presence of a hypothetic flow cell which could be located for example below the first substrate. The adjusted thickness of the first substrate can simulate the collective effects of the first substrate and the hypothetical flow cell containing a fluid/liquid. The disclosure also provides flow cells comprising pluralities of fluorescent beads for use as fiducials, and methods of using same in high throughput sequencing applications.

### CROSS REFERENCE TO RELATED APPLICATIONS

This application is a continuation of International Application No. PCT/IB2024/059112, filed on Sep. 19, 2024, which claims priority to, and benefit of, U.S. Provisional Application No. 63/584,056 filed on Sep. 20, 2023, the contents of each of which are incorporated by reference in their entirety herein.

### TECHNICAL FIELD

The present disclosure provides apparatus and methods for evaluating the performance of optical imaging systems included in sequencing systems, using solid-state and configurable optical test targets and flow cell devices. The present disclosure also provides apparatus and methods for image registration of flow cell images during sequencing using the flow cell devices.

### BACKGROUND

Many types of technologies, including next generation sequencing (NGS) technologies, use image analysis of samples in flow cells. In an exemplary NGS protocol, DNA template molecules are clonally amplified on a surface of the flow cell to generate polonies. The polonies on the flow cell are then sequenced in parallel through progressive rounds of hybridization of labeled bases to the template molecule, or incorporation of labeled bases in extension products, followed by imaging of the labeled bases to identify the corresponding base identity in the template molecule. There thus exists a need for systems and methods that can enable accurate imaging, and image registration, of flow cells through repeating rounds of base addition and imaging during NGS.

### SUMMARY

The disclosure provides solid-state optical test targets comprising: (a) a first substrate comprising a transparent medium, and having top surface, bottom surface and one or more side surfaces, the top and bottom surfaces being flat, and the first substrate having a refractive index of [n-top substrate(1)]; and (b) a second substrate having top surface, bottom surface and side surfaces, the top surface being flat, wherein (i) at least a portion of the top surface of the second substrate comprises an opaque coating that forms a micropattern, the micropattern configured to include opaque portions and transparent portions, (ii) the first substrate is positioned on top of the second substrate, and the first substrate is positioned in direct contact with the micropattern on the second substrate, (iii) the solid-state optical test target lacks a flow cell and lacks a liquid, (iv) the thickness of the first substrate simulates the presence of a first hypothetical flow cell located between the first and second substrates, wherein the first hypothetical flow cell includes a first channel having a top surface and bottom surface, the first channel containing a first fluid, wherein the first channel has a first thickness of [T-channel(1)] and the first fluid has a refractive index of [n-fluid(1)], and (v) the thickness of the first substrate is configured to permit imaging of the bottom surface of the first channel of the first hypothetical flow cell.

In some embodiments of the solid-state optical test targets of the disclosure, the top surface, bottom surface and one or more side surfaces have an even thickness. In some embodiments, the first substrate and/or second substrate comprise transparent glass. In some embodiments, the top, side and/or bottom surfaces of the second substrate are transparent to permit light transmission through the top, side and/or bottom surfaces. In some embodiments, the thickness of the opaque coating that forms the micropattern on the top surface of the second substrate is about 100 nm. In some embodiments, the opaque coating comprises chromium or aluminum. In some embodiments, the transparent portions of the micropattern comprise regions of the top surface of the second substrate without the opaque coating. In some embodiments, the transparent portions of the micropattern comprise repeating shapes arranged in an array. In some embodiments, the transparent portions of the micropattern form a plurality of one type of shape, or a mixture of different types of shapes, and the one type of shape and the mixture of different types of shapes are selected from a group consisting of circles, squares and triangles. In some embodiments, the transparent portions of the micropattern forms the shape of at least one line. In some embodiments, the transparent portions of the micropattern form at least one alphanumeric character. In some embodiments, the transparent portions of the micropattern form a plurality of pinholes, and the plurality of pinholes fill a full field of view when the solid-state optical test target is positioned in an optical imaging system which comprises at least one objective lens, at least one tube lens, and a camera. In some embodiments, the transparent portions of the micropattern form a non-repeating rotationally symmetrical shape including concentric circles. In some embodiments, the symmetrical shape comprises a bullseye or a plus sign (+). In some embodiments, the dimension of the transparent portions of the micropattern is about 1 micron. In some embodiments, the opaque portions of the micropattern comprising repeating shapes arranged in an array. In some embodiments, the opaque portions of the micropattern form a plurality of one type of shape, or a mixture of different types of shapes, where the one type of shape and the mixture of different types of shapes are selected from a group consisting of circles, squares and triangles. In some embodiments, the opaque portions of the micropattern form the shape of at least one line. In some embodiments, the opaque portions of the micropattern form at least one alphanumeric character. In some embodiments, the opaque portions of the micropattern form a plurality of pinholes. In some embodiments, the plurality of pinholes fill a full field of view when the solid-state optical test target is positioned in an optical imaging system which comprises at least one objective lens, at least one tube lens, and a camera. In some embodiments, the opaque portions of the micropattern form a non-repeating rotationally symmetrical shape including concentric circles. In some embodiments, the symmetrical shape comprises a bullseye or plus sign (+). In some embodiments, the dimension of the opaque portions of the micropattern is about 1 micron. In some embodiments, the height/thickness of the first substrate [T-top substrate(1)] is related to the refractive index of the first substrate [n-top substrate(1)], the first height of the first channel [T-channel(1)] and the refractive index of the first fluid [n-fluid(1)], in an equation

$$[ T - top substrate ( 1 ) ] = ( [ ( T - channel ( 1 ) ] * ( [ ( n - fluid ( 1 ) ] / [ n - top substrate ( 1 ) ] ) ) . \qquad ( Equation 2 )$$

In some embodiments of the solid-state optical test targets of the disclosure, the optical test target is positioned in an optical imaging system which comprises at least one objective lens, at least one tube lens, and a camera. In some embodiments, the optical imaging system further comprises at least on light source and at least one filter. In some embodiments, the at least on light source comprises a laser or LED excitation light. In some embodiments, the at least one light source is positioned to excite a fluorophore in a sample.

In some embodiments of the solid-state optical test targets of the disclosure, the first substrate is removable from the second substrate. In some embodiments, the first substrate is replaced with a third substrate which comprises a transparent medium and having a top surface, a bottom surface and one or more side surfaces, and the third substrate having a refractive index of [n-top substrate(3)], and (i) the third substrate is positioned on top of the second substrate, and the third substrate is positioned in direct contact with the micropattern on the second substrate, (ii) the solid-state optical test target lacks a flow cell and lacks a liquid, (iii) the height (thickness) of the third substrate is configured to simulate the presence of a second hypothetical flow cell located between the third and second substrates, the second hypothetical flow cell includes a second channel having a top surface and a bottom surface, and the second channel containing a second fluid, the second channel has a second thickness of [T-channel(2)] and the second fluid has a refractive index of [n-fluid(2)], and (iv) the thickness of the third substrate is configured to permit imaging of the bottom surface of the second channel of the second hypothetical flow cell. In some embodiments, the top surface, bottom surface and one or more side surfaces have an even thickness. In some embodiments, the third substrate comprises transparent glass. In some embodiments, the height/thickness of the third substrate [T-top substrate(3)] is related to the refractive index of the third substrate [n-top substrate(3)], the second height of the second channel [T-channel(2)] and the refractive index of the second fluid [n-fluid(2)], in an equation

$$[ T - top substrate ( 3 ) ] = ( [ ( T - channel ( 2 ) ] * ( [ ( n - fluid ( 2 ) ] / [ n - top substrate ( 3 ) ] ) ) . \qquad ( Equation 3 )$$

In some embodiments of the solid-state optical test targets of the disclosure, the refractive index of the first substrate [n-top substrate(1)] is the same or different from the refractive index of the third substrate [n-top substrate(3)]. In some embodiments, the height of the first hypothetical channel [T-channel(1)] is the same or different from the height of the second hypothetical channel [T-channel(2)]. In some embodiments, the refractive index of the first fluid [n-fluid(1)] is the same or different from the refractive index of the second fluid [n-fluid(2)]. In some embodiments, the solid-state optical test target is positioned in an optical imaging system which comprises at least one objective lens, at least one tube lens, and a camera. In some embodiments, the optical imaging system further comprises at least on light source and at least one filter. In some embodiments, the bottom surface of the second substrate comprises a reflecting coating, or the bottom surface of the second substrate comprises a rough scatter surface. In some embodiments, the second substrate comprises a first fluorescent microscope slide that provides a continuous fluorescent field and comprises a material that has first fluorescence spectrum. In some embodiments, first fluorescence spectrum comprises a green band emission. In some embodiments, the solid-state optical test target comprises a second fluorescent microscope slide located under the first fluorescent microscope slide, and the second fluorescent microscope slide provides a continuous fluorescent field and has a second fluorescence spectrum that differs or does not substantially overlap with the first fluorescence spectrum of the first fluorescent microscope slide. In some embodiments, the second fluorescent microscope slide has a fluorescence spectrum that produces a red band emission. In some embodiments, the second substrate comprises a planar-shaped LED light.

The disclosure provides adaptive solid-state optical test targets comprising: (a) a first substrate comprising a transparent medium and a top surface, a bottom surface and one or more side surfaces, the first substrate having at least two regions comprising different thicknesses, wherein the first region and has a first thickness and the second region and has a second thickness, and the first substrate comprises a refractive index of [n-top substrate(1)]; and (b) a second substrate comprising a top surface, a bottom surface and one or more side surfaces, the top surface being flat, wherein (i) at least a portion of the top surface of the second substrate comprises an opaque coating that forms a micropattern, wherein the micropattern is configured to include opaque portions and transparent portions, (ii) the first substrate is positioned on top of the second substrate, and the first substrate is positioned in direct contact with the micropattern on the second substrate, (iii) the solid-state optical test target lacks a flow cell and lacks a liquid, (iv) the thickness of the first region of the first substrate is configured to simulate the presence of a first hypothetical flow cell located between the first and second substrates, wherein the first hypothetical flow cell includes a first channel having a top surface and a bottom surface, and the first channel comprises a first fluid, wherein the first channel has a first thickness of [T-channel(1)] and the first fluid has a refractive index of [n-fluid(1)], (v) the thickness of the first region of the first substrate is configured to permit imaging of the bottom surface of the first channel of the first hypothetical cell, and (v) the thickness of the second region of the first substrate is configured to permit imaging of the top surface of the first channel of the first hypothetical cell.

In some embodiments of the adaptive solid-state optical test targets of the disclosure, the bottom surface is flat. In some embodiments, the first and second regions are flat. In some embodiments, the transparent medium comprises transparent glass. In some embodiments, the second substrate comprises transparent glass. In some embodiments, the top surface, one or more side surfaces and/or bottom surface of the second substrate are transparent to permit light transmission through the top surface, one or more side surfaces and/or bottom surface. In some embodiments, the thickness of the opaque coating that forms the micropattern on the top surface of the second substrate is about 100 nm. In some embodiments, the opaque coating comprises chromium or aluminum. In some embodiments, the transparent portions of the micropattern comprise repeating shapes arranged in an array. In some embodiments, the transparent portions of the micropattern form a plurality of one type of shape, or a mixture of different types of shapes, where the one type of shape and the mixture of different types of shapes are selected from a group consisting of circles, squares and triangles. In some embodiments, the transparent portions of the micropattern forms the shape of at least one line. In some embodiments, the transparent portions of the micropattern form at least one alphanumeric character. In some embodiments, the transparent portions of the micropattern comprise regions of the top surface of the second substrate without the opaque coating. In some embodiments, the transparent portions of the micropattern form a plurality of pinholes, and the plurality of pinholes fill a full field of view when the solid-state optical test target is positioned in an optical imaging system which comprises at least one objective lens, at least one tube lens, and a camera. In some embodiments, the transparent portions of the micropattern form a non-repeating rotationally symmetrical shape including concentric circles. In some embodiments, the symmetrical shape comprises a bullseye or plus sign (+). In some embodiments, the dimension of the transparent portions of the micropattern is about 1 micron. In some embodiments, the opaque portions of the micropattern comprising repeating shapes arranged in an array. In some embodiments, the opaque portions of the micropattern form a plurality of one type of shape, or a mixture of different types of shapes, where the one type of shape and the mixture of different types of shapes are selected from a group consisting of circles, squares and triangles. In some embodiments, the opaque portions of the micropattern forms the shape of at least one line. In some embodiments, the opaque portions of the micropattern form at least one alphanumeric character. In some embodiments, the opaque portions of the micropattern form a plurality of pinholes such that the pinholes fill a full field of view when the solid-state optical test target is positioned in an optical imaging system which comprises at least one objective lens, at least one tube lens, and a camera. In some embodiments, the opaque portions of the micropattern form a non-repeating rotationally symmetrical shape including concentric circles. In some embodiments, the symmetrical shape comprises a bullseye or plus sign (+). In some embodiments, the dimension of the opaque portions of the micropattern is about 1 micron. In some embodiments, the height/thickness of the first region of the first substrate [T-top substrate(1)] is related to the refractive index of the first substrate [n-top substrate(1)], the first designated height of the first channel [T-channel(1)] and the refractive index of the first designated fluid [n-fluid(1)], in an equation

$$[ T - top substrate ( 1 ) ] = ( [ ( T - channel ( 1 ) ] * ( [ ( n - fluid ( 1 ) ] / [ n - top substrate ( 1 ) ] ) ) . \qquad ( Equation 2 )$$

In some embodiments of the adaptive solid-state optical test targets of the disclosure, the adaptive solid-state optical test target is positioned in an optical imaging system which comprises at least one objective lens, at least one tube lens, and a camera. In some embodiments, the optical imaging system further comprises at least on light source and at least one filter. In some embodiments, the at least on light source comprises a laser or LED excitation light. In some embodiments, the at least one light source is positioned to excite a fluorophore in a sample.

The disclosure provides adaptive solid-state optical test targets comprising: (a) a substrate comprising a transparent medium with a top surface, a bottom surface and one or more side surfaces, the substrate having at least two regions with different thicknesses, wherein the first region has a first thickness and the second region has a second thickness, and the substrate comprises a refractive index of [n-top substrate(1)]; and (b) the substrate comprises at least one layer of fluorescent dye layered on the bottom surface of the substrate, wherein (i) at least a portion of the bottom surface of the substrate comprises an opaque coating that forms a micropattern, the micropattern configured to include opaque portions and transparent portions, (ii) the at least one fluorescent dye layer is layered on the opaque coating such that the opaque coating is disposed between the bottom surface of the substrate and the at least one fluorescent dye layer, (iii) the adaptive solid-state optical test target lacks a flow cell and lacks a liquid, (iv) the thickness of the first region of the substrate is configured to simulate the presence of a first hypothetical flow cell located between the first and second substrates, wherein the first hypothetical flow cell includes a first channel having a top surface and a bottom surface, the first channel containing a first fluid, wherein the first channel has a first thickness of [T-channel(1)] and the first fluid has a refractive index of [n-fluid(1)], (v) the thickness of the first region of the substrate is configured to permit imaging of the bottom surface of the first channel of the first hypothetical flow cell, and (vi) the thickness of the second region of the substrate is configured to permit imaging of the top surface of the first channel of the first hypothetical flow cell.

In some embodiments of the adaptive solid-state optical test targets of the disclosure, the bottom surface is flat. In some embodiments, the first and second regions are flat. In some embodiments, the transparent medium comprises transparent glass. In some embodiments, the thickness of the opaque coating that forms the micropattern on the bottom surface of the substrate is about 100 nm. In some embodiments, the opaque coating comprises chromium or aluminum. In some embodiments, the transparent portions of the micropattern comprise repeating shapes arranged in an array. In some embodiments, the transparent portions of the micropattern form a plurality of one type of shape, or a mixture of different types of shapes, where the one type of shape and the mixture of different types of shapes are selected from a group consisting of circles, squares and triangles. In some embodiments, the transparent portions of the micropattern forms the shape of at least one line. In some embodiments, the transparent portions of the micropattern form at least one alphanumeric character. In some embodiments, the transparent portions of the micropattern comprise regions of the top surface of the second substrate without the opaque coating. In some embodiments, the transparent portions of the micropattern form a plurality of pinholes, and the plurality of pinholes fill a full field of view when the solid-state optical test target is positioned in an optical imaging system which comprises at least one objective lens, at least one tube lens, and a camera. In some embodiments, the transparent portions of the micropattern form a non-repeating rotationally symmetrical shape including concentric circles. In some embodiments, the symmetrical shape comprises a bullseye or plus sign (+). In some embodiments, the dimension of the transparent portions of the micropattern is about 1 micron. In some embodiments, the opaque portions of the micropattern comprising repeating shapes arranged in an array. In some embodiments, the opaque portions of the micropattern form a plurality of one type of shape, or a mixture of different types of shapes, where the one type of shape and the mixture of different types of shapes are selected from a group consisting of circles, squares and triangles. In some embodiments, the opaque portions of the micropattern forms the shape of at least one line. In some embodiments, the opaque portions of the micropattern form at least one alphanumeric character. In some embodiments, the opaque portions of the micropattern form a plurality of pinholes, and the plurality of pinholes fill a full field of view when the solid-state optical test target is positioned in an optical imaging system which comprises at least one objective lens, at least one tube lens, and a camera. In some embodiments, the opaque portions of the micropattern form a non-repeating rotationally symmetrical shape including concentric circles. In some embodiments, the symmetrical shape comprises a bullseye or plus sign (+). In some embodiments, the dimension of the opaque portions of the micropattern is about 1 micron. In some embodiments, the height/thickness of the first region of the substrate [T-top substrate(1)] is related to the refractive index of the substrate [n-top substrate(1)], the first designated height of the first channel [T-channel(1)] and the refractive index of the first designated fluid [n-fluid(1)], in an equation

$$[ T - top substrate ( 1 ) ] = ( [ ( T - channel ( 1 ) ] * ( [ ( n - fluid ( 1 ) ] / [ n - top substrate ( 1 ) ] ) ) . \qquad ( Equation 2 )$$

In some embodiments of the adaptive solid-state optical test targets of the disclosure, the adaptive solid-state optical test target further comprises a second layer of fluorescent dye layered on the first layer of fluorescent dye. In some embodiments, the first layer of fluorescent dye comprises a fluorescent dye having an excitation spectrum of 520-540 nm which can produce a green band emission of 530-630 nm. In some embodiments, the first layer of fluorescent dye comprises a mixture of two fluorescent dyes. In some embodiments, the first fluorescent dye comprises an excitation spectrum of 520-540 nm which can produce a green band emission of 530-630 nm, and the second fluorescent dye comprises an excitation spectrum of 630-650 nm which can produce a red band emission of 630-690 nm. In some embodiments, the first layer of fluorescent dye comprises a first fluorescent dye having an excitation spectrum of 520-540 nm which can produce a green band emission of 530-630 nm, and the second layer of fluorescent dye comprises a second fluorescent dye having an excitation spectrum of 630-650 nm which can produce a red band emission of 630-690 nm.

In some embodiments, the adaptive solid-state optical test target is positioned in an optical imaging system which comprises at least one objective lens, at least one tube lens, and a camera. In some embodiments, the optical imaging system further comprises at least on light source and at least one filter. In some embodiments, the at least on light source comprises a laser or LED excitation light. In some embodiments, the at least one light source is positioned to excite a fluorophore in a sample.

The disclosure provides fluorescent solid-state optical test targets comprising: (a) a substrate comprising a transparent medium comprising a top surface, a bottom surface and one or more side surfaces, wherein at least a portion of the bottom surface of the substrate comprises an opaque coating that forms a micropattern, the micropattern configured to include opaque portions and transparent portions, and the substrate having a refractive index of [n-top substrate(1)]; and (b) at least one layer of fluorescent dyes layered on the bottom surface of the substrate where the fluorescent dye layer is layered on the opaque coating, wherein (i) the fluorescent solid-state optical test target lacks a flow cell and lacks a liquid, (ii) the thickness of the substrate is configured to simulate the presence of a first hypothetical flow cell located between the first and second substrates, wherein the first hypothetical flow cell includes a first channel having a top surface and bottom surface, and the first channel containing a designated first fluid, wherein the first channel has a first designated thickness of [T-channel(1)] and the first designated fluid has a refractive index of [n-fluid(1)], and (iii) the thickness of the first substrate is configured to permit imaging of the bottom surface of the first channel of the first hypothetical flow cell.

In some embodiments of the fluorescent solid-state optical test targets of the disclosure, the top surface, bottom surface and one or more side surfaces have an even thickness. In some embodiments, the top surface and the bottom surface are flat. In some embodiments, the transparent medium comprises transparent glass. In some embodiments, the thickness of the opaque coating that forms the micropattern on the bottom surface of the substrate is about 100 nm. In some embodiments, the opaque coating comprises chromium or aluminum. In some embodiments, the transparent portions of the micropattern comprise repeating shapes arranged in an array. In some embodiments, the transparent portions of the micropattern form a plurality of one type of shape, or a mixture of different types of shapes, where the one type of shape and the mixture of different types of shapes are selected from a group consisting of circles, squares and triangles. In some embodiments, the transparent portions of the micropattern forms the shape of at least one line. In some embodiments, the transparent portions of the micropattern form at least one alphanumeric character. In some embodiments, the transparent portions of the micropattern comprise regions of the bottom surface of the substrate without the opaque coating. In some embodiments, the transparent portions of the micropattern form a plurality of pinholes such that the pinholes fill a full field of view when the solid-state optical test target is positioned in an optical imaging system which comprises at least one objective lens, at least one tube lens, and a camera. In some embodiments, the transparent portions of the micropattern form a non-repeating rotationally symmetrical shape including concentric circles. In some embodiments, the symmetrical shape comprises a bullseye or plus sign (+). In some embodiments, the dimension of the transparent portions of the micropattern is about 1 micron. In some embodiments, the opaque portions of the micropattern comprising repeating shapes arranged in an array. In some embodiments, the opaque portions of the micropattern form a plurality of one type of shape, or a mixture of different types of shapes, where the one type of shape and the mixture of different types of shapes are selected from a group consisting of circles, squares and triangles. In some embodiments, the opaque portions of the micropattern forms the shape of at least one line. In some embodiments, the opaque portions of the micropattern form at least one alphanumeric character. In some embodiments, the opaque portions of the micropattern form a plurality of pinholes such that the pinholes fill a full field of view when the solid-state optical test target is positioned in an optical imaging system which comprises at least one objective lens, at least one tube lens, and a camera. In some embodiments, opaque portions of the micropattern form a non-repeating rotationally symmetrical shape including concentric circles. In some embodiments, the symmetrical shape comprises a bullseye or plus sign (+). In some embodiments, the dimension of the opaque portions of the micropattern is about 1 micron. In some embodiments, the height/thickness of the first region of the substrate [T-top substrate(1)] is related to the refractive index of the substrate [n-top substrate(1)], the first designated height of the first channel [T-channel(1)] and the refractive index of the first designated fluid [n-fluid(1)], in an equation

$$[ T - top substrate ( 1 ) ] = ( [ ( T - channel ( 1 ) ] * ( [ ( n - fluid ( 1 ) ] / [ n - top substrate ( 1 ) ] ) ) . \qquad ( Equation 2 )$$

In some embodiments of the fluorescent solid-state optical test targets of the disclosure, the fluorescent solid-state optical test target further comprises a second layer of fluorescent dye layered on the first layer of fluorescent dye. In some embodiments, the first layer of fluorescent dyes comprises a fluorescent dye having an excitation spectrum of 520-540 nm which can produce a green band emission of 530-630 nm. In some embodiments, the first layer of fluorescent dyes comprises a mixture of two fluorescent dyes. In some embodiments, the first fluorescent dye having an excitation spectrum of 520-540 nm which can produce a green band emission of 530-630 nm, and the second fluorescent dye having an excitation spectrum of 630-650 nm which can produce a red band emission of 630-690 nm. In some embodiments, the first layer of fluorescent dye comprises a first fluorescent dye having an excitation spectrum of 520-540 nm which can produce a green band emission of 530-630 nm, and the second layer of fluorescent dye comprises a second fluorescent dye having an excitation spectrum of 630-650 nm which can produce a red band emission of 630-690 nm.

In some embodiments of the fluorescent solid-state optical test targets of the disclosure, the fluorescent solid-state optical test target is positioned in an optical imaging system which comprises at least one objective lens, at least one tube lens, and a camera. In some embodiments, the optical imaging system further comprises at least on light source and at least one filter. In some embodiments, the at least on light source comprises a laser or LED excitation light. In some embodiments, the at least one light source is positioned to excite a fluorophore in a sample.

The disclosure provides methods for evaluating the performance of an optical imaging system, comprising the steps: (a) positioning the solid-state optical test target of the disclosure in an optical imaging system comprising at least one objective lens, at least one tube lens, a camera, at least on light source and at least one filter; (b) detecting light transmitted through the first substrate; and (c) evaluating the performance of the optical imaging system based on the light that is transmitted through the first substrate.

In some embodiments of the methods for evaluating the performance of an optical imaging system of the disclosure, the methods comprise the steps: (a) positioning the adaptive solid-state optical test target of the disclosure in an optical imaging system comprising at least one objective lens, at least one tube lens, a camera, at least on light source and at least one filter; (b) detecting light transmitted through the third substrate; and (c) evaluating the performance of the optical imaging system based on the light that is transmitted through the third substrate.

The disclosure provides methods for evaluating the performance of an optical imaging system, comprising the steps: (a) positioning the solid-state optical test target of the disclosure in an optical imaging system which comprises at least one objective lens, at least one tube lens, a camera, at least on light source and at least one filter; (b) detecting light transmitted through the substrate; and (c) evaluating the performance of the optical imaging system based on the light that is transmitted through the substrate.

The disclosure provides methods for evaluating the performance of an optical imaging system, comprising the steps: (a) positioning the adaptive solid-state optical test target of the disclosure in an optical imaging system comprising at least one objective lens, at least one tube lens, a camera, at least on light source and at least one filter; (b) detecting light transmitted through the first region of the first substrate; (c) detecting light transmitted through the second region of the first substrate; and (d) evaluating the performance of the optical imaging system based on the light that is transmitted through the first and second regions of the first substrate.

The disclosure provides methods for evaluating the performance of an optical imaging system, comprising the steps: (a) positioning the adaptive solid-state optical test target of the disclosure in an optical imaging system comprising at least one objective lens, at least one tube lens, a camera, at least on light source and at least one filter; (b) detecting light transmitted through the first region of the first substrate; (c) detecting light transmitted through the second region of the first substrate; and (d) evaluating the performance of the optical imaging system based on the light that is transmitted through the first and second regions of the first substrate.

The disclosure provides methods for evaluating the performance of an optical imaging system, comprising the steps: (a) positioning the fluorescent solid-state optical test target of the disclosure in an optical imaging system comprising at least one objective lens, at least one tube lens, a camera, at least on light source and at least one filter; (b) detecting light transmitted through the substrate; and (c) evaluating the performance of the optical imaging system based on the light that is transmitted through the substrate.

In some embodiments of the methods of the disclosure, the at least on light source comprises a laser or LED excitation light. In some embodiments, the at least one light source is positioned to excite a fluorophore in a sample. In some embodiments, evaluating the performance of the optical imaging system comprises any one or more of: (i) determining the accuracy of the optical alignment; (ii) determining the autofocus accuracy; (iii) calibrating the light source; (iv) calibrating the camera; (v) determining an image uniformity correction; (vi) determining distortion levels; (vii) determining contrast across a field of view; (viii) determining alignment of the camera (sensor); (ix) determining the focal distance of the camera (sensor); (x) determining flat field correction; (xi) determining focus repeatability; (xii) determining point spread function measurement; and/or (xiii) determining a modulated transfer function (MTF).

The disclosure provides flow cell devices comprising: a support comprising one or more substrates comprising one or more channels; an inlet in the one or more substrates; and an outlet in the one or more substrates, wherein the one or more channels run from the inlet to the outlet, and wherein the one or more channels comprise a surface coated with a plurality of fluorescent beads that are immobilized to the surface.

In some embodiments of the flow cell devices of the disclosure, the plurality of fluorescent beads are chemically immobilized to the surface. In some embodiments, the surface is passivated. In some embodiments, the surface is passivated with a coating that can immobilize polynucleotides. In some embodiments, the polynucleotides comprise surface capture primers, nucleic acid template molecules, or both. In some embodiments, the surface comprises a plurality of polynucleotides captured thereon. In some embodiments, the flow cell device is configured to simultaneous image the polynucleotides and the plurality fluorescent beads in a first sequencing cycle via a first channel using a sequencing system. In some embodiments, the polynucleotides are imaged in a first sequencing cycle via a first channel, and the plurality of fluorescent beads are imaged in a second sequencing cycle via a second channel using a sequencing system. In some embodiments, the first cycle is not a dark cycle and the second cycle is a dark cycle. In some embodiments, the plurality of fluorescent beads comprise one, two, three, four, five or six different types of beads, and each type of bead emits a different color or combination of colors in response to excitement by a laser. In some embodiments, the flow cell devices comprises an about equal amount of the two, three, four, five, or six different types of beads. In some embodiments, at least a portion of the polynucleotides moves relative to the surface and the plurality fluorescent beads remain immobilized relative to the surface from a first sequencing cycle to a second sequencing cycle in a sequencing run. In some embodiments, at least a portion of the polynucleotides move from first positions in a first flow cell image acquired via a first channel in a first sequencing cycle to second positions in a second flow cell image acquired via a second channel in the first sequencing cycle, and the plurality of fluorescent beads remain immobilized relative to the surface in the first and second flow cell images.

In some embodiments of the flow cell devices of the disclosure, the flow cell device is configured to be used on a sequencing system for calibrating the sequencing system. In some embodiments, the sequencing system comprises one, two, three, four, five, or six color channels, and the flow cell devised is used for calibrating the one, two, three, four, five, or six color channels of the sequencing system. In some embodiments, the flow cell device enables image registration of images of the polynucleotides taken on a sequencing system between different sequencing cycles or color channels of the same sequencing cycle, or a combination thereof, and the image registration is based on the relative positions of the plurality of fluorescent beads.

In some embodiments of the flow cell devices of the disclosure, the one or more substrates comprises a top substrate and a bottom substrate. In some embodiments, the one or more channels are defined between the top substrate and the bottom substrate. In some embodiments, the surface is an interior top surface, an interior bottom surface, or both, of the one or more channels.

In some embodiments of the flow cell devices of the disclosure, the plurality of fluorescent beads emit a first fluorescent light in response to laser excitement in a first sequencing cycle in a first sequencing run. In some embodiments, the first fluorescent light comprises a first wavelength, a first intensity, a first color, or a combination thereof. In some embodiments, the plurality of fluorescent beads emit a second fluorescent light in response to laser excitement in an additional sequencing cycle in the first sequencing run. In some embodiments, the additional sequencing cycle is a 100th cycle, a 110th cycle, a 120th cycle, or a 130th cycle. In some embodiments, the second fluorescent light comprises a second wavelength, a second intensity, a second color, or a combination thereof. In some embodiments, the second intensity is less than about 10%, 8%, or 5% different from the first intensity. In some embodiments, the plurality of fluorescent beads emit a third fluorescent light in response to laser excitement in a first sequencing cycle in a second sequencing run after storage of the flow cell device. In some embodiments, the storage comprises about 6 months at about room temperature. In some embodiments, the third fluorescent light comprises a third wavelength, a third intensity, a third color, or a combination thereof. In some embodiments, the third intensity is less than about 10%, 8%, or 5% different from the first intensity. In some embodiments, the plurality of fluorescent beads emit a fourth fluorescent light in response to laser excitement in a first sequencing cycle in a third sequencing run after exposing the flow cell device for about 30 minutes to an about 100° C. environment after the first sequencing run. In some embodiments, the fourth fluorescent light comprises a fourth wavelength, a fourth intensity, a fourth color, or a combination thereof. In some embodiments, the fourth intensity is about less than 10%, 8%, or 5% different from the first intensity. In some embodiments, the plurality of fluorescent beads emit a fifth fluorescent light in response to laser excitement in a first sequencing cycle in a fourth sequencing run after drying the flow cell device and refilling the flow cell with reagents at least once, twice, 5 times, 10 times, or 15 times after the first sequencing run. In some embodiments, the flow cell has been dried and refilled more than 20 times after the first sequencing run. In some embodiments, the fifth fluorescent light comprises a fifth wavelength, a fifth intensity, a fifth color, or a combination thereof. In some embodiments, the fifth intensity is less than about 10%, 8%, or 5% different from the first intensity.

In some embodiments of the flow cell devices of the disclosure, two or more of the first, second, third, fourth, and fifth wavelengths are about identical. In some embodiments, the first fluorescent light is obtained from a first channel, and the plurality of fluorescent beads emit sixth fluorescent light in response to laser excitement in the first sequencing cycle in a second channel in the first sequencing run. In some embodiments, the sixth fluorescent light comprises a sixth wavelength, a sixth intensity, a sixth color, or a combination thereof. In some embodiments, the sixth intensity is about less than 10%, 8%, or 5% different from the first intensity. In some embodiments, two or more of the first, second, third, fourth, fifth, and sixth wavelengths are about identical. In some embodiments, the first and the sixth wavelengths are different, and the first and the sixth colors are different. In some embodiments, the first, second, third, fourth, fifth, or sixth wavelength is within a range from about 150 nm to about 850 nm. In some embodiments, the first, second, third, fourth, fifth, or sixth color is red, green, blue, yellow, or a combination thereof. In some embodiments, two or more of the first, second, third, fourth, fifth, and sixth colors are about identical.

In some embodiments of the flow cell devices of the disclosure, the fluorescent beads are about randomly distributed on the surface. In some embodiments, an imaging area on the surface comprises about 150,000 to about 450,000 fluorescent beads. In some embodiments, an imaging area comprises at least a portion of a subtile of the flow cell device. In some embodiments, the fluorescent beads comprise microspheres loaded with fluorescent dyes. In some embodiments, the microspheres comprise a diameter of about 0.1 μm to about 1.0 um. In some embodiments, the fluorescent beads comprise quantum dots. In some embodiments, the one or more substrates comprise glass or plastic.

In some embodiments of the flow cell devices of the disclosure, the first, second, or sixth wavelength, the first, second, or sixth color, the first, second, or sixth intensity, or combinations thereof are configured to enable image registration of polynucleotides imaged using a sequencing system between different sequencing cycles or between different color channels. In some embodiments, the first, second, sixth wavelength, the first, second, or sixth color, the first, second, or sixth intensity, or combinations thereof are configured to enable calibration of a sequencing system.

In some embodiments of the flow cell devices of the disclosure, the fluorescent beads are covalently attached to the surface. In some embodiments, at least a portion of the polynucleotides move from first positions in a first flow cell image acquired via a first channel in a first sequencing cycle to second positions in a second flow cell image acquired via a second channel in a second sequencing cycle, the fluorescent beads in the plurality remain immobilized relative to the surface in the first and second flow cell images, and the first sequencing cycle and the second sequencing cycle are different. In some embodiments, at least a portion of the polynucleotides move from first positions in a first flow cell image acquired via a first channel in a first sequencing cycle to second positions in a second flow cell image acquired via a second channel in a second sequencing cycle, the fluorescent beads in the plurality remain immobilized relative to the surface in the first and second flow cell images, the first sequencing cycle and the second sequencing cycle are different and the first channel and the second channel are identical.

In some embodiments of the flow cell devices of the disclosure, at least part of the support is transparent. In some embodiments, at least part of the one or more substrates is transparent. In some embodiments, the support is solid.

In some embodiments of the flow cell devices of the disclosure, an intensity of some or all of the fluorescent beads are less than 50%, 40%, 30%, 20%, or 10% different from an intensity of some or all of polynucleotides in flow cell images obtained from a same channel. In some embodiments, the one or more channels comprises 1, 2, 3, 4, 5, 6, 7, or 8 channels. In some embodiments, a first sequencing cycle and a second sequencing cycle are a same sequencing cycle. In some embodiments, a first sequencing cycle and a second sequencing cycle are different sequencing cycles. In some embodiments, the first channel and the second channel are a same channel. In some embodiments, the first channel and the second channel are a different channel.

The disclosure provides methods of calibrating a sequencing system, the methods comprising: generating first flow cell images by imaging the flow cell device of the disclosure using a sequencing system in a first sequencing cycle using a first channel; generating second flow cell images by imaging the flow cell device using the sequencing system in a second sequencing cycle using a second channel; and calibrating the sequencing system by analyzing the first flow cell images and the second flow cell images.

In some embodiments of the methods of calibrating a sequencing system of the disclosure, the first and second sequencing cycle are a same cycle. In some embodiments, the first and second sequencing cycle are different cycles. In some embodiments, the first and second channel are a same channel. In some embodiments, the first and second channel are different channels.

The disclosure provides methods of performing image registration of flow cell images, the methods comprising: generating first images by imaging the flow cell device of the disclosure using a sequencing system in a first sequencing cycle using a first channel; generating second images by imaging the flow cell device using the sequencing system in a second sequencing cycle using a second channel; and performing image registration by analyzing the first flow cell images and the second flow cell images.

In some embodiments of the methods of performing image registration of flow cell images of the disclosure, the first and second sequencing cycle are a same cycle. In some embodiments, the first and second sequencing cycle are different cycles. In some embodiments, the first and second channel are a same channel. In some embodiments, the first and second channel are different channels.

The disclosure provides methods of performing image registration of flow cell images, the methods comprising: generating first images by imaging the flow cell device of the disclosure using a sequencing system in a first sequencing cycle using a first channel; generating second images by imaging the flow cell device using the sequencing system in a second sequencing cycle using a second channel; and performing image registration based on positions of fluorescent beads and positions of polynucleotides of the flow cell device in the first flow cell images and the second flow cell images.

In some embodiments of the methods of performing image registration of flow cell images of the disclosure, the first and second sequencing cycle are a same cycle. In some embodiments, the first and second sequencing cycle are different cycles. In some embodiments, the first and second channel are a same channel. In some embodiments, the first and second channel are different channels.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic representation of a solid-state optical test target and a hypothetical flow cell, according to an embodiment. The Left side of FIG. 1 shows the solid-state optical test target which includes a first substrate (also referred to as a top substrate), a second substrate (also referred to as a bottom substrate), and an opaque layer disposed between the first (top) and second (bottom) substrate. The opaque layer can be coated on a lower and/or bottom surface of the first (top) substrate. Alternatively, in some implementations the opaque layer can be coated on an upper and/or top surface of the second (bottom) substrate. The opaque layer forms a micropattern. The top substrate, or a portion thereof, can be made of a transparent material which permits incident light to penetrate the top substrate (as shown in FIG. 1) and be transmitted from its bottom surface, facilitating view of the micropattern. The solid-state optical test target lacks a flow cell and liquid. The thickness of the first substrate can be adjusted to simulate the effect of a hypothetic flow cell which contains a fluid/liquid on the transmission of light through the solid-state optical test target, when the hypothetical flow cell is disposed between the top and the bottom substrates. The Right side of FIG. 1 shows a hypothetical flow cell which includes a channel having a thickness (the T-channel), with the channel containing a fluid/liquid having a refractive index (n-fluid). The solid-state optical test target shown in FIG. 1 can be positioned on an optical imaging system and used to evaluate the performance of the optical imaging system by obtaining image information about the top surface of the hypothetical flow cell channel.

FIG. 2 is a schematic representation of a solid-state optical test target and a hypothetical flow cell, according to an embodiment. The Left side of FIG. 2 shows the solid-state optical test target, which includes a first substrate (top substrate), a second substrate (bottom substrate), and an opaque layer disposed between the first (top) substrate and second (bottom) substrate. The opaque layer can be coated on a lower and/or bottom surface of the first (top) substrate or on an upper and/or top surface of the second (bottom) substrate. The opaque layer forms a micropattern. The top substrate, or a portion thereof, can be made of a transparent material which permits incident light to penetrate the top substrate (as shown in FIG. 2) and be transmitted from its bottom surface, facilitating a view of the micropattern. The solid-state optical test target lacks a flow cell and liquid. The thickness of the first substrate can be adjusted to simulate the effect of a hypothetic flow cell which contains a fluid/liquid on the transmission of light through the solid-state optical test target, when the hypothetical flow cell is disposed between the top and the bottom substrates. For example, the top substrate can be thicker, having an add-on thickness (T-top substrate, shown in FIG. 2) which simulates the light transmission effects of a hypothetical flow cell disposed between the top and the bottom substrates. The Right side of FIG. 2 shows a hypothetical flow cell which includes a channel having a thickness (the T-channel) and the channel contains a fluid/liquid having a refractive index (n-fluid). The solid-state optical test target shown in FIG. 2 can be positioned on an optical imaging system and used to evaluate the performance of the optical imaging system by obtaining image information about the bottom surface of the hypothetical flow cell channel.

FIG. 3 is a schematic representation of an exemplary solid-state optical test target with an exchangeable top substrate, according to an embodiment. The Left side of FIG. 3 shows a solid-state optical test target having a first substrate (also referred to as an original top substrate), a second substrate (also referred to as an original bottom substrate), and an opaque layer disposed between the first and the second substrates. The opaque layer can be coated on a lower and/or bottom surface of the original top substrate or on an upper and/or top surface of the original bottom substrate. The opaque layer forms a micropattern. The original top substrate, or a portion thereof, is made of a transparent material which permits incident light to penetrate the original top substrate (as shown in FIG. 3) and be transmitted from its bottom surface, facilitating a view of the micropattern. The solid-state optical test target lacks a flow cell and liquid. The thickness of the original top substrate can be adjusted (e.g., by adding an add-on thickness as shown in FIG. 3) to simulate the effect of a first hypothetic flow cell which contains a fluid/liquid on the transmission of light through the solid-state optical test target, when a first hypothetical flow cell is located between the original top substrate and the original bottom substrate. FIG. 3 shows the original top substrate and the add-on thickness, which can be collectively referred to as a T-top substrate(1), which can be replaced by a replacement top substrate with a respective add-on thickness (e.g., the T-top substrate(3) as shown on the Right side of FIG. 3). The replacement T-top substrate(3) can be positioned on the original bottom substrate, where the replacement T-top substrate(3) is thicker than the T-top substrate(1), and the add-on thickness of the replacement substrate T-top substrate(3) can be adjusted to simulate the effect of a second hypothetic flow cell which contains a fluid/liquid on the transmission of light through the solid-state optical test target, when the second hypothetical flow cell is located between the replacement top substrate and the original bottom substrate. The solid-state optical test targets shown in FIG. 3 can be positioned on an optical imaging system and used to evaluate the performance of the optical imaging system by obtaining image information about the bottom surface of the hypothetical flow cell channel. In some embodiments, the replacement T-top substrate(3) may be thicker than the original T-top substrate(1). The add-on thickness of the replacement T-top substrate(3) may be greater than zero. In some embodiments, alone or in combination with using the replacement T-top substrate(3), the solid-state optical test target may have a replacement bottom substrate. The replacement bottom substrate can be positioned underneath the original top substrate or the replacement T-top substrate(3).

FIG. 4 is a schematic representation of an exemplary solid-state optical test target including a first substrate (top substrate), an opaque (micropattern) layer, and two fluorescent dye layers (e.g., a 1^{st} fluorescent dye layer and a 2^{nd} fluorescent dye layer), according to an embodiment. The opaque (micropattern) layer is coated on a lower and/or bottom surface of the top substrate. The opaque layer forms a micropattern. The top substrate, or a portion thereof, can be made of a transparent material which permits incident light to penetrate the top substrate and be transmitted from its bottom surface, facilitating a view of the micropattern. The solid-state optical test target lacks a flow cell and liquid. The thickness of the top substrate can be adjusted to simulate the effect of a hypothetic flow cell which contains a fluid/liquid on the transmission of light through the solid-state optical test target, when the hypothetical flow cell is located between the top substrate and the fluorescent dye layers. The solid-state optical test targets shown in FIG. 4 can be positioned on an optical imaging system and used to evaluate the performance of the optical imaging system by obtaining image information about the bottom surface of the hypothetical flow cell channel.

FIG. 5A is a schematic representation of an exemplary adaptive solid-state optical test target having a first substrate (top substrate), a second substrate (bottom substrate), and an opaque layer disposed between the top substrate and the bottom substrate, where the top substrate has at least two regions with different thicknesses, according to an embodiment. The bottom substrate can comprise a transparent glass substrate. The opaque layer can be coated on a lower and/or bottom surface of the top substrate or an upper and/or top surface of the bottom substrate. The opaque layer forms a micropattern. The top substrate, or a portion thereof, can be made of a transparent material which permits incident light to penetrate the top substrate and be transmitted from its bottom surface, facilitating view of the micropattern. The solid-state optical test target lacks a flow cell and liquid. At least one of the regions of the top substrate has a thickness that is configured to simulate the effect of a first hypothetical flow cell which contains a fluid/liquid on the transmission of light through the solid-state optical test target (Add-on thickness, producing the T-top substrate), when the first hypothetical flow cell is located between the top substrate and bottom substrate. For example, one of the regions of the top substrate can be thicker, having an add-on thickness. The solid-state optical test targets shown in FIG. 5A can be positioned on an optical imaging system and used to evaluate the performance of the optical imaging system by obtaining image information about the bottom surface of the hypothetical flow cell channel.

FIG. 5B is a schematic representation of an exemplary adaptive solid-state optical test target having a first substrate (top substrate), at least one of a 1^{st} fluorescent dye layer and/or a 2^{nd} fluorescent dye layer, and an opaque layer between the top substrate and the fluorescent dye layer(s), where the top substrate has at least two regions with different thicknesses. The opaque layer can be coated on a lower and/or bottom surface of the top substrate. The opaque layer forms a micropattern. The top substrate, or a portion thereof, can be made of a transparent material which permits incident light to be transmitted from its bottom surface, facilitating a view of the micropattern. The solid-state optical test target lacks a flow cell and liquid. At least one of the regions of the top substrate has a thickness that is configured to simulate the effect of a first hypothetical flow cell which contains a fluid/liquid on the transmission of light through the solid-state optical test target, when the hypothetical flow cell is located between the top substrate and the fluorescent dye layer(s). For example, one of the regions of the top substrate is thicker, having an add-on thickness (the add-on thickness and top substrate are referred to collectively as the T-top substrate). The solid-state optical test targets shown in FIG. 5B can be positioned on an optical imaging system and used to evaluate the performance of the optical imaging system by obtaining image information about the bottom surface of the hypothetical flow cell channel.

FIG. 6 is a schematic representation of an exemplary solid-state optical test target having a first substrate (top substrate), a fluorescent microscope slide, and an opaque layer disposed between the top substrate and the fluorescent microscope slide. The opaque layer can be coated on a lower and/or bottom surface of the top substrate. The opaque layer forms a micropattern. The top substrate, or a portion thereof, can be made of a transparent material which permits incident light to be transmitted from its bottom surface, facilitating a view of the micropattern. The solid-state optical test target lacks a flow cell and liquid. The thickness of the top substrate can be adjusted to simulate the effect of a hypothetic flow cell which contains a fluid/liquid on the transmission of light through the solid-state optical test target, when the hypothetical flow cell is located between the top substrate and the fluorescent microscope slide (top substrate and add-on thickness, referred to collectively as the T-top substrate). The solid-state optical test targets shown in FIG. 6 can be positioned on an optical imaging system and used to evaluate the performance of the optical imaging system by obtaining image information about the bottom surface of the hypothetical flow cell channel.

FIG. 7 is a schematic showing an exemplary solid-state optical test target having a first substrate (top substrate), a light emitting diode (LED) light, and an opaque layer disposed between the top substrate and the LED light. The LED light can be a planar LED light. The opaque layer can be coated on a lower and/or bottom surface of the top substrate. The opaque layer forms a micropattern. The first substrate is transparent which permits light transmission from its bottom surface and a view of the micropattern. The solid-state optical test target lacks a flow cell and liquid. The thickness of the top substrate can be adjusted to simulate the effect of a hypothetic flow cell which contains a fluid/liquid on the transmission of light through the solid-state optical test target, when the hypothetical flow cell is located between the top substrate and the LED light (top substrate and add-on thickness, referred to collectively as the T-top substrate). The solid-state optical test targets shown in FIG. 7 can be positioned on an optical imaging system and used to evaluate the performance of the optical imaging system by obtaining image information about the bottom surface of the hypothetical flow cell channel.

FIG. 8 is a fluorescence image recorded with an optical imaging system when a solid-state optical test target having a first substrate (top substrate), an LED light, and an opaque layer between the top substrate and the LED light (see FIG. 7) is positioned on the optical imaging system. FIG. 8 shows an opaque chromium layer and pinhole arrays, which were prepared using a semi-conductor manufacturing process. The pinholes are 0.5 um in diameter and are positioned 30 microns apart. The solid-state optical test target shown in FIG. 8 was used to evaluate sub-resolution transmissive pinholes for point spread function measurements and distortion. The pinholes filled the field of view. Slanted edges over the full field of view were disposed for modulated transfer function (MTF). A multi-channel alignment (camera alignment) was used to record images, an image from the red channel is shown at left, and an image from the green channel is shown at right. The high density of pinholes permitted measurements of contrast across field of view. This optical test target was used to align tip/tilt and focal distance of sensors. Optical transmission was determined to be dependent on wavelength and opaque layer, e.g., chromium layer, thickness. The opaque layer thickness was selected to be >3 OD for all wavelengths. A unique, non-repeating structure such as concentric circles (e.g., bulls-eye) or a plus character (+) will allow rotation and translation of cameras to be corrected.

FIG. 9 shows a schematic drawing of an exemplary flow cell device having a first surface coated with a 1^{st} coating of fluorescent beads and a second surface coated with a 2^{nd} coating of fluorescent beads. The coatings of fluorescence beads can be directly applied on the substrates of a solid support of the flow cell device. The flow cell device with the coating(s) can be positioned on a sequencing system for performing or facilitating sequencing analysis by obtaining and analyzing images of the fluorescent beads.

FIG. 10 shows images of an exemplary flow cell device disclosed herein obtained using a sequencing system. The images are from the same sequencing run at a first sequencing cycle (left) and at a 100th sequencing cycle (right).

FIG. 11 shows fluorescence images of an exemplary flow cell device disclosed herein obtained using a sequencing system in a first sequencing run (left), and in another sequencing run, after loading the flow device with water and drying the flow cell device for 5 times using vacuum drying (middle), in yet another sequencing run, after loading and drying the flow cell device for 20 times (right).

FIG. 12 shows images of an exemplary flow cell device disclosed herein obtained using a sequencing system in a first sequencing run at day 0 (left), in another sequencing run, after storing the flow cell device at room temperature for 41 days (middle), and in yet another sequencing run, after storing the flow cell device for 220 days (right).

FIG. 13 shows an image of an exemplary flow cell device disclosed herein obtained using a sequencing system in a sequencing run after exposing the flow cell device to a 100° C. environment for about 30 minutes.

FIG. 14 illustrates a block diagram of a computer-implemented system for performing DNA sequencing and sequencing analysis, according to some embodiments.

FIG. 15 is a schematic showing an exemplary linear single stranded library molecule (700) according to an embodiment, which comprises: a surface pinning primer binding site (720); an optional left unique identification sequence (780); a left index sequence (760); a forward sequencing primer binding site (740); an insert region having a sequence of interest (710); reverse sequencing primer binding site (750); a right index sequence (770); and a surface capture primer binding site (730).

FIG. 16 is a schematic showing an exemplary linear single stranded library molecule (700) according to an embodiment, which comprises: a surface pinning primer binding site (720); a left index sequence (760); a forward sequencing primer binding site (740); an insert region having a sequence of interest (710); a reverse sequencing primer binding site (750); a right index sequence (770); an optional right unique identification sequence (790); and a surface capture primer binding site (730).

FIG. 17 is a schematic of various exemplary configurations of multivalent molecules. Left (Class I): schematics of multivalent molecules having a “starburst” or “helter-skelter” configuration. Center (Class II): a schematic of a multivalent molecule having a dendrimer configuration. Right (Class III): a schematic of multiple multivalent molecules formed by reacting streptavidin with 4-arm or 8-arm PEG-NHS with biotin and dNTPs. Nucleotide units are designated ‘N’, biotin is designated ‘B’, and streptavidin is designated ‘SA’.

FIG. 18 is a schematic of an exemplary multivalent molecule comprising a generic core attached to a plurality of nucleotide-arms.

FIG. 19 is a schematic of an exemplary multivalent molecule comprising a dendrimer core attached to a plurality of nucleotide-arms.

FIG. 20 shows a schematic of an exemplary multivalent molecule comprising a core attached to a plurality of nucleotide-arms, where the nucleotide arms comprise biotin, spacer, linker and a nucleotide unit.

FIG. 21 is a schematic of an exemplary nucleotide-arm comprising a core attachment moiety, spacer, linker and nucleotide unit.

FIG. 22 shows the chemical structure of an exemplary spacer (top), and the chemical structures of various exemplary linkers, including an 11-atom Linker, 16-atom Linker, 23-atom Linker and an N3 Linker (bottom).

FIG. 23 shows the chemical structures of various exemplary linkers, including Linkers 1-9.

FIG. 24 shows the chemical structures of various exemplary linkers joined/attached to nucleotide units.

FIG. 25 shows the chemical structures of various exemplary linkers joined/attached to nucleotide units.

FIG. 26 shows the chemical structures of various exemplary linkers joined/attached to nucleotide units.

FIG. 27 shows the chemical structures of various exemplary linkers joined/attached to nucleotide units.

FIG. 28 shows the chemical structure of an exemplary biotinylated nucleotide-arm. In this example, the nucleotide unit is connected to the linker via a propargyl amine attachment at the 5 position of a pyrimidine base or the 7 position of a purine base.

FIGS. 29A-29B show a central portion of an exemplary solid-state optical test target disclosed herein with a pinhole array and a center cross.

FIGS. 29C-29D show exemplary solid-state optical test targets, in this case, relative to the LED light in the excitation path of the optical test target.

FIG. 30 shows an exemplary image of the optical test target in FIGS. 29A-29B detected using the optical imaging system disclosed herein. In this case, the image shows spatial offsets of pinholes and center cross acquired from different color channels indicating a need for alignment of different color channels of the optical imaging system.

In the drawings, like reference numbers generally indicate identical or similar elements. Additionally, generally, the left-most digit(s) of a reference number identifies the drawing in which the reference number first appears.

### DETAILED DESCRIPTION

### Definitions

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative term (e.g., “or”) is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include: “A and B”; “A or B”; “A” (A alone); and “B” (B alone). In a similar manner, the term “and/or” as used in a phrase such as “A, B, and/or C” is intended to encompass each of the following aspects: “A, B, and C”; “A, B, or C”; “A or C”; “A or B”; “B or C”; “A and B”; “B and C”; “A and C”; “A” (A alone); “B” (B alone); and “C” (C alone).

As used herein and in the appended claims, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the terms “about,” “approximately,” and “substantially” refer to a value or composition that is within an acceptable error range for the particular value or composition as determined by one of ordinary skill in the art, which will depend in part on how the value or composition is measured or determined, i.e., the limitations of the measurement system. For example, “about,” “approximately,” or “substantially” can mean within one or more than one standard deviation per the practice in the art. Alternatively, “about” or “approximately” can mean a range of up to 10% (i.e., +10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about,” “approximately,” “substantially” should be assumed to be within an acceptable error range for that particular value or composition. Also, where ranges and/or subranges of values are provided, the ranges and/or subranges can include the endpoints of the ranges and/or subranges.

The term “glass” refers to silica-based material, including silicate, borosilicate, fused silica, fused quartz, glass, quartz, or lead glass.

The term “array” refers to a plurality of sites (the sites can be any shape, e.g. pinholes) located at pre-determined locations on an opaque layer on the substrate to form an array of sites. The sites can be discrete and separated by interstitial regions. The pre-determined sites on the substrate can be arranged in one dimension in a row or a column, or arranged in two dimensions in rows and columns. The plurality of pre-determined sites can be arranged on the substrate in an organized fashion, for example in any organized pattern, including rectilinear, hexagonal patterns, grid patterns, patterns having reflective symmetry, patterns having rotational symmetry, or the like. The pitch between any two sites can be that same or can vary. In some embodiments, the substrate comprises at least 2 sites, at least 10 sites, at least 20 sites, at least 40 sites, at least 60 sites, at least 80 sites, at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, wherein the sites are located at pre-determined locations on the substrate.

The term “flow cell” or “flow cell device” refers to a support employed in next generation sequencing (NGS) methods. The support can be solid. At least some portion of the support can be transparent, or all of the support can be transparent. The support can include a multi-layer structure fabricated from substrates and other flow cell components which are then bonded through mechanical, chemical, or laser bonding techniques to form fluid flow channels. For example, the support can be multiple layers of glass plates or substrates fabricated together. The glass plate or substrate can have a pre-determined thickness. The support can have a plurality of oligonucleotide surface primers immobilized thereon. The support can have surface capture primers, nucleic acid template molecules, or both attached or immobilized thereon. In some embodiments, the support is passivated with a low non-specific binding coating. The surface coatings can exhibit very low non-specific binding to reagents typically used for nucleic acid capture, amplification and sequencing workflows, such as dyes, nucleotides, enzymes, and nucleic acid primers. The surface coatings can exhibit low background fluorescence signals or high contrast-to-noise (CNR) ratios compared to conventional surface coatings.

The term “nucleotides” and related terms refers to a molecule comprising an aromatic base, a five-carbon sugar (e.g., ribose or deoxyribose), and at least one phosphate group. Canonical or non-canonical nucleotides are consistent with use of the term. The phosphate in some embodiments comprises a monophosphate, diphosphate, or triphosphate, or corresponding phosphate analog. In some embodiments, the nucleotide comprises 1, 2, 3, 4, 5, 6, 7, 8, 9 or 10 phosphate groups. The term “nucleoside” refers to a molecule comprising an aromatic base and a sugar.

Nucleotides (and nucleosides) typically comprise a hetero cyclic base including substituted or unsubstituted nitrogen-containing parent heteroaromatic ring which are commonly found in nucleic acids, including naturally-occurring, substituted, modified, or engineered variants, or analogs of the same. The base of a nucleotide (or nucleoside) is capable of forming Watson-Crick and/or Hoogstein hydrogen bonds with an appropriate complementary base. Exemplary bases include, but are not limited to, purines and pyrimidines such as: 2-aminopurine, 2,6-diaminopurine, adenine (A), ethenoadenine, N⁶-Δ²-isopentenyladenine (6iA), N⁶-Δ²-isopentenyl-2-methylthioadenine (2ms6iA), N⁶-methyladenine, guanine (G), isoguanine, N²-dimethylguanine (dmG), 7-methylguanine (7 mG), 2-thiopyrimidine, 6-thioguanine (6sG), hypoxanthine and O⁶-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O⁴-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional exemplary bases can be found in Fasman, 1989, in “Practical Handbook of Biochemistry and Molecular Biology”, pp. 385-394, CRC Press, Boca Raton, Fla.

Nucleotides (and nucleosides) typically comprise a sugar moiety, such as carbocyclic moiety (Ferraro and Gotor 2000 Chem. Rev. 100:4319-48), acyclic moieties (Martinez, et al., 1999 Nucleic Acids Research 27:1271-1274; Martinez, et al., 1997 Bioorganic & Medicinal Chemistry Letters vol. 7:3013-3016), and other sugar moieties (Joeng, et al., 1993 J. Med. Chem. 36:2627-2638; Kim, et al., 1993 J. Med. Chem. 36:30-7; Eschenmosser 1999 Science 284:2118-2124; and U.S. Pat. No. 5,558,991). The sugar moiety comprises: ribosyl; 2′-deoxyribosyl; 3′-deoxyribosyl; 2′,3′-dideoxyribosyl; 2′,3′-didehydrodideoxyribosyl; 2′-alkoxyribosyl; 2′-azidoribosyl; 2′-aminoribosyl; 2′-fluororibosyl; 2′-mercaptoriboxyl; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some embodiments, nucleotides comprise a chain of one, two or three phosphorus atoms where the chain is typically attached to the 5′ carbon of the sugar moiety via an ester or phosphoramide linkage. In some embodiments, the nucleotide is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

The terms “nucleic acid”, “polynucleotide” and “oligonucleotide” and other related terms used herein are used interchangeably and refer to polymers of nucleotides, and are not limited to any particular length. Nucleic acids include recombinant and chemically-synthesized forms. Nucleic acids include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and chimeric forms containing DNA and RNA. Nucleic acids can be single-stranded or double-stranded. Nucleic acids comprise polymers of nucleotides, where the nucleotides include natural or non-natural bases and/or sugars. Nucleic acids comprise naturally-occurring internucleosidic linkages, for example phosphdiester linkages. Nucleic acids can also comprise non-natural internucleosidic linkages, including phosphorothioate, phosphorothiolate, or peptide nucleic acid (PNA) linkages. Nucleic acids can comprise a mixture of naturally-occurring internucleosidic linkages, and non-natural internucleosidic linkages in the same nucleic acid molecule. In some embodiments, nucleic acids comprise a one type of polynucleotides or a mixture of two or more different types of polynucleotides.

The term “primer” and related terms used herein refer to an oligonucleotide, either natural or synthetic, that is capable of hybridizing with a DNA and/or RNA polynucleotide template to form a duplex molecule. The skilled artisan will appreciate that primers can be fully complementary to the DNA and/or RNA polynucleotide template to which they hybridize, or contain one or more mismatches, but still be capable of hybridizing with the DNA and/or RNA template. Primers may have any length, but typically range from 4-50 nucleotides. A typical primer comprises a 5′ end and 3′ end. The 3′ end of the primer can include a 3′ OH moiety which serves as a nucleotide polymerization initiation site in a polymerase-mediated primer extension reaction. Alternatively, 3′ end of the primer can lack a 3′ OH moiety, or can include a terminal 3′ blocking group that inhibits nucleotide polymerization in a polymerase-mediated reaction. Any one nucleotide, or more than one nucleotide, along the length of the primer can be labeled with a detectable reporter moiety. A primer can be in solution (e.g., a soluble primer) or can be immobilized to a support (e.g., a capture primer).

The term “template nucleic acid”, “template polynucleotide”, “target nucleic acid” “target polynucleotide”, “template strand” and other variations refer to a nucleic acid strand that serves as the basis nucleic acid molecule for generating a complementary nucleic acid strand. The template nucleic acid can be single-stranded or double-stranded, or the template nucleic acid can have single-stranded or double-stranded portions. The sequence of the template nucleic acid can be partially or wholly complementary to the sequence of the complementary strand. The template nucleic acid can be obtained from a naturally-occurring source, recombinant form, or chemically synthesized to include any type of nucleic acid analog. The template nucleic acid can be linear, circular, or other forms. The template nucleic acids can include an insert region having an insert sequence which is also known as a “sequence of interest,” “target sequence” or “target sequence of interest.” The template nucleic acids can also include at least one adaptor sequence. The template nucleic acid can be a concatemer having two or more tandem copies of a sequence of interest and at least one adaptor sequence. The insert region can be isolated from any samples in any suitable form, including samples of chromosomal, genomic, organellar (e.g., mitochondrial, chloroplast or ribosomal) nucleic acids, recombinant molecules, cloned, amplified, cDNA, RNA such as precursor mRNA or mRNA, oligonucleotides, whole genomic DNA, nucleic acids obtained from fresh frozen or paraffin embedded tissue, needle biopsies, cell free circulating DNA, or any type of nucleic acid library. The insert region can be isolated from any source including from organisms such as prokaryotes, eukaryotes (e.g., humans, plants and animals), fungus, viruses cells, tissues, cells (e.g. single cells), normal or diseased cells or tissues, body fluids including blood, urine, serum, lymph, tumor, saliva, anal and vaginal secretions, amniotic samples, perspiration, semen, environmental samples, culture samples, or synthesized nucleic acid molecules prepared using recombinant molecular biology or chemical synthesis methods. The insert region can be isolated from any organ, including head, neck, brain, breast, ovary, cervix, colon, rectum, endometrium, gallbladder, intestines, bladder, prostate, testicles, liver, lung, kidney, esophagus, pancreas, thyroid, pituitary, thymus, skin, heart, larynx, or other organs. The template nucleic acid can be subjected to nucleic acid analysis, including sequencing and composition analysis.

The term “polony” or “PCR colony” used herein refers to a nucleic acid library molecule that has been clonally amplified in-solution or on-support to generate an amplicon that can serve as a template molecule for sequencing. In an exemplary polony, a linear library molecule can be circularized to generate a circularized library molecule, and the circularized library molecule can be clonally amplified in-solution or on-support to generate a concatemer. This concatemer can serve as a nucleic acid template molecule which can be sequenced, and is sometimes referred to as a polony. A polony can include nucleotide strands. Alternatively, a polony can refer to a plurality of library molecules, fixed in position on a substrate, that have been generated by amplification of a single parental molecule.

As used herein, the term “sequencing” and its variants refer to methods that comprise obtaining sequence information from a nucleic acid strand, typically by determining the identity of at least some nucleotides (including their nucleobase components) within the nucleic acid template molecule. “Sequencing” can refer to sequencing a given region of a nucleic acid molecule, and include identifying each and every nucleotide within the region that is sequenced. Alternatively, “sequencing” comprises methods whereby the identity of only some of the nucleotides in the region is determined, while the identity of some nucleotides remains undetermined or incorrectly determined. Any suitable method of sequencing may be used. For example, sequencing can include label-free or ion based sequencing methods. As a further example, sequencing can include labeled or dye-containing nucleotide or fluorescent based nucleotide sequencing methods. Sequencing can include polony-based sequencing or bridge sequencing methods. Sequencing also includes massively parallel sequencing platforms that employ sequence-by-synthesis, sequence-by-hybridization or sequence-by-binding procedures. Examples of massively parallel sequence-by-synthesis procedures include polony sequencing, pyrosequencing (e.g., from 454 Life Sciences; U.S. Pat. Nos. 7,211,390, 7,244,559 and 7,264,929), chain-terminator sequencing (e.g., from Illumina; U.S. Pat. No. 7,566,537; Bentley 2006 Current Opinion Genetics and Development 16:545-552; and Bentley, et al., 2008 Nature 456:53-59, ion-sensitive sequencing (e.g., from Ion Torrent), probe-anchor ligation sequencing (e.g., Complete Genomics), DNA nanoball sequencing, nanopore DNA sequencing. Examples of single molecule sequencing include Heliscope single molecule sequencing, and single molecule real time (SMRT) sequencing from Pacific Biosciences (Levene, et al., 2003 Science 299 (5607): 682-686; Eid, et al., 2009 Science 323 (5910): 133-138; U.S. Pat. Nos. 7,170,050; 7,302,146; and 7,405,281). An example of sequence-by-hybridization includes SOLID sequencing (e.g., from Life Technologies; WO 2006/084132). An example of sequence-by-binding includes Omniome sequencing (e.g., U.S. Pat. No. 10,246,744).

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules that have not yet been subjected to post-translation modification such as proteolytic cleavage, cleavage due to ribosomal skipping, hydroxylation, methylation, lipidation, acetylation, SUMOylation, ubiquitination, glycosylation, phosphorylation and/or disulfide bond formation. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins.

The term “polymerase” and its variants, as used herein, encompasses any enzyme that can catalyze polymerization of nucleotides (including analogs thereof) into a nucleic acid strand. Typically, but not necessarily, such nucleotide polymerization can occur in a template-dependent fashion. Typically, a polymerase comprises one or more active sites at which nucleotide binding and/or catalysis of nucleotide polymerization can occur. Polymerases can also include other enzymatic activities, such as for example, 3′ to 5′ exonuclease activity or 5′ to 3′ exonuclease activity. For example, some polymerases have strand displacing activity. A polymerase can include, without limitation, naturally occurring polymerases and any subunits, functional fragments or truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze nucleotide polymerization (e.g., catalytically active fragment). Polymerases can be isolated from cells, or generated using recombinant DNA technology or chemical synthesis methods. Polymerases can be expressed in prokaryote, eukaryote, viral, or phage organisms. A polymerase can be a post-translationally modified protein or fragment thereof. A polymerase can be derived from a prokaryote, eukaryote, virus or phage. A polymerase can comprise DNA-directed DNA polymerase activity, and/or RNA-directed DNA polymerase activity.

As used herein, the term “fidelity” refers to the accuracy of DNA polymerization by a template-dependent DNA polymerase. The fidelity of a DNA polymerase is typically measured by the error rate (the frequency of incorporating an inaccurate nucleotide, i.e., a nucleotide that is not complementary to the corresponding nucleotide in the template molecule). The accuracy or fidelity of DNA polymerization is maintained by both the polymerase activity and 3′-5′ exonuclease activity of a DNA polymerase.

When used in reference to nucleic acids, the terms “extend”, “extending”, “extension” and other variants, refers to incorporation of one or more nucleotides into a nucleic acid molecule. Nucleotide incorporation comprises polymerization of one or more nucleotides into the terminal 3′ OH end of a nucleic acid strand, resulting in extension of the nucleic acid strand. Nucleotide incorporation can be conducted with natural nucleotides and/or nucleotide analogs. Typically, but not necessarily, nucleotide incorporation occurs in a template-dependent fashion. Any suitable method of extending a nucleic acid molecule may be used, including primer extension catalyzed by a DNA polymerase or RNA polymerase.

As used herein, the term “binding complex” refers to a complex formed by binding together a nucleic acid duplex, a polymerase, and a free nucleotide or a nucleotide unit (or moiety) of a multivalent molecule, where the nucleic acid duplex comprises a nucleic acid template molecule hybridized to a nucleic acid primer. In the binding complex, the free nucleotide or nucleotide unit may or may not be bound to the 3′ end of the nucleic acid primer at a position that is opposite a complementary nucleotide in the nucleic acid template molecule. A “ternary complex” is an example of a binding complex which is formed by binding together a nucleic acid duplex, a polymerase, and a free nucleotide or nucleotide unit of a multivalent molecule, where the free nucleotide or nucleotide unit is bound to the 3′ end of the nucleic acid primer (as part of the nucleic acid duplex) at a position that is opposite a complementary nucleotide in the nucleic acid template molecule.

The term “persistence time” and related terms refer to the length of time that a binding complex remains stable without dissociation of any of the components. For example, a binding complex where the components of the binding complex include a nucleic acid template and nucleic acid primer, a polymerase, a nucleotide unit of a multivalent molecule or a free (e.g., unconjugated) nucleotide. The nucleotide unit or the free nucleotide can be complementary or non-complementary to a nucleotide residue in the template molecule. The nucleotide unit or the free nucleotide can bind to the 3′ end of the nucleic acid primer at a position that is opposite a complementary nucleotide residue in the nucleic acid template molecule. The persistence time is indicative of the stability of the binding complex and strength of the binding interactions. Persistence time can be measured by observing the onset and/or duration of a binding complex, such as by observing a signal from a labeled component of the binding complex. For example, a labeled nucleotide or a labeled reagent comprising one or more nucleotides may be present in a binding complex, thus allowing the signal from the label to be detected during the persistence time of the binding complex. One exemplary label is a fluorescent label. In some cases, the binding complex (e.g., ternary complex) remains stable until subjected to a condition that causes dissociation of interactions between any of its members, for example the polymerase, template molecule, primer and/or the nucleotide unit or the nucleotide. For example, a dissociating condition comprises contacting the binding complex with any one or any combination of a detergent, EDTA and/or water.

When used in reference to nucleic acid molecules, the terms “hybridize” or “hybridizing” or “hybridization” or other related terms refers to hydrogen bonding between two different nucleic acids to form a duplex nucleic acid. Hybridization also includes hydrogen bonding between two different regions of a single nucleic acid molecule to form a self-hybridizing molecule having a duplex region. Hybridization can comprise Watson-Crick or Hoogstein binding to form a duplex double-stranded nucleic acid, or a double-stranded region within a nucleic acid molecule. The double-stranded nucleic acid, or the two different regions of a single nucleic acid, may be wholly complementary, or partially complementary. Complementary nucleic acid strands need not hybridize with each other across their entire length. The complementary base pairing can be the standard A-T or C-G base pairing, or can be other forms of base-pairing interactions. Duplex nucleic acids can include mismatched base-paired nucleotides.

The terms “linked”, “joined”, “attached”, and variants thereof comprise any type of fusion, bond, adherence or association between any combination of substrates, compounds or molecules that is of sufficient stability to withstand use in the particular procedure. The procedure can include but are not limited to: nucleotide transient-binding; nucleotide incorporation; de-blocking; washing; removing; flowing; detecting; imaging and/or identifying. Such linkage can comprise, for example, covalent, ionic, hydrogen, dipole-dipole, hydrophilic, hydrophobic, or affinity bonding, bonds or associations involving van der Waals forces, mechanical bonding, and the like. In some embodiments, such linkage occurs intramolecularly, for example linking together the ends of a single-stranded or double-stranded linear nucleic acid molecule to form a circular molecule. In some embodiments, such linkage can occur between a combination of different molecules, or between a molecule and a non-molecule, including but not limited to: linkage between a nucleic acid molecule and a solid surface; linkage between a protein and a detectable reporter moiety; linkage between a nucleotide and detectable reporter moiety; and the like. Some examples of linkages can be found, for example, in Hermanson, G., “Bioconjugate Techniques”, Second Edition (2008); Aslam, M., Dent, A., “Bioconjugation: Protein Coupling Techniques for the Biomedical Sciences”, London: Macmillan (1998); Aslam, M., Dent, A., “Bioconjugation: Protein Coupling Techniques for the Biomedical Sciences”, London: Macmillan (1998). In the context of a device such as a flow cell, the various components of the device that are in close physical proximity (e.g., touching each other, or touching other components which touch each other), such as the top, bottom and walls, or surfaces and their coatings, can be termed to be linked, joined or attached.

The term “operably linked” and “operably joined” or related terms as used herein refers to juxtaposition of components. The juxtapositioned components can be linked by any suitable means known in the art. For example, two molecules can be linked together covalently. For example, two nucleic acid components can be enzymatically ligated together where the linkage that joins together the two components comprises phosphodiester linkage. A first and second nucleic acid component can be linked together, where the first nucleic acid component can confer a function on a second nucleic acid component. For example, linkage between a primer binding sequence and a sequence of interest forms a nucleic acid library molecule having a portion that can bind to a primer. In another example, a transgene (e.g., a nucleic acid encoding a polypeptide or a nucleic acid sequence of interest) can be ligated to a vector where the linkage permits expression or functioning of the transgene sequence contained in the vector. In some embodiments, a transgene is operably linked to a host cell regulatory sequence (e.g., a promoter sequence) that affects expression of the transgene. In some embodiments, the vector comprises at least one host cell regulatory sequence, including a promoter sequence, enhancer, transcription and/or translation initiation sequence, transcription and/or translation termination sequence, polypeptide secretion signal sequences, and the like. In some embodiments, the host cell regulatory sequence controls expression of the level, timing and/or location of the transgene.

The term “adaptor” and related terms refers to oligonucleotides that can be operably linked (appended) to a target polynucleotide, wherein the adaptor confers a function to the co-joined adaptor-target molecule. Adaptors comprise DNA, RNA, chimeric DNA/RNA, or analogs thereof. Adaptors can include at least one ribonucleoside residue. Adaptors can be single-stranded, double-stranded, or have single-stranded and/or double-stranded portions. Adaptors can be configured to be linear, stem-looped, hairpin, or Y-shaped forms. Adaptors can be any length, including 4-100 nucleotides or longer. Adaptors can have blunt ends, overhang ends, or a combination of both. Overhang ends include 5′ overhang and 3′ overhang ends. The 5′ end of a single-stranded adaptor, or one strand of a double-stranded adaptor, can have a 5′ phosphate group or lack a 5′ phosphate group. Adaptors can include a 5′ tail that does not hybridize to a target polynucleotide (e.g., tailed adaptor), or adaptors can be non-tailed. An adaptor can include a sequence that is complementary to at least a portion of a primer, such as an amplification primer, a sequencing primer, or a capture primer (e.g., soluble or immobilized capture primers). Adaptors can include a random sequence or degenerate sequence. Adaptors can include at least one inosine residue. Adaptors can include at least one phosphorothioate, phosphorothiolate and/or phosphoramidate linkage. Adaptors can include a barcode sequence which can be used to distinguish polynucleotides (e.g., insert sequences) from different sample sources in a multiplex assay. Adaptors can include a unique identification sequence (e.g., unique molecular index, UMI; or a unique molecular tag) that can be used to uniquely identify a nucleic acid molecule to which the adaptor is appended. In some embodiments, a unique identification sequence can be used to increase error correction and accuracy, reduce the rate of false-positive variant calls and/or increase sensitivity of variant detection. Adaptors can include at least one restriction enzyme recognition sequence, including any one or any combination of two or more selected from a group consisting of type I, type II, type III, type IV, type Hs or type IIB.

The term “universal sequence”, “universal adaptor sequences” and related terms refers to a sequence in a nucleic acid molecule that is common among two or more polynucleotide molecules. For example, adaptors having the same universal sequence can be joined to a plurality of polynucleotides so that the population of co-joined molecules carry the same universal adaptor sequence. Examples of universal adaptor sequences include an amplification primer sequence, a sequencing primer sequence or a capture primer sequence (e.g., soluble or support-immobilized capture primers).

The term “branched polymer” and related terms refers to a polymer having a plurality of functional groups that help conjugate a biologically active molecule such as a nucleotide, and the functional group can be either on the side chain of the polymer or directly attaches to a central core or central backbone of the polymer. The branched polymer can have linear backbone with one or more functional groups coming off the backbone for conjugation. The branched polymer can also be a polymer having one or more sidechains, wherein the side chain has a site suitable for conjugation. Examples of the functional group include but are limited to hydroxyl, ester, amine, carbonate, acetal, aldehyde, aldehyde hydrate, alkenyl, acrylate, methacrylate, acrylamide, active sulfone, hydrazide, thiol, alkanoic acid, acid halide, isocyanate, isothiocyanate, maleimide, vinylsulfone, dithiopyridine, vinylpyridine, iodoacetamide, epoxide, glyoxal, dione, mesylate, tosylate, and tresylate.

As used herein, the term “clonally amplified” and it variants refers to a nucleic acid template molecule that has been subjected to one or more amplification reactions either in-solution or on-support. In the case of in-solution amplified template molecules, the resulting amplicons are distributed onto the support. Prior to amplification, the template molecule comprises a sequence of interest and at least one universal adaptor sequence. In some embodiments, clonal amplification comprises the use of a polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification (RCA), circle-to-circle amplification, helicase-dependent amplification, recombinase-dependent amplification, single-stranded binding (SSB) protein-dependent amplification, or any combination thereof.

The term “flow cell” as used herein refers to a chamber comprising a solid surface across which one or more fluid reagents can be flowed. Examples of flow cells and related fluidic systems and detection platforms are described, for example, in U.S. Pat. No. 11,028,435, the contents of which are incorporated herein by reference.

As used herein, a “hypothetical flow cell” refers to a flow cell used in applications such as next generation sequencing whose physical properties (components and their dimensions, index of refraction, light transmission when imaging, and the like) are known to the person of ordinary skill in the art, such that the behavior of this hypothetical flow cell in an optical imaging system can be mimicked by the configuration of the optical test target. For example, the thickness of a substrate the optical test target can be configured by adjusting its thickness to simulate the presence of the hypothetical flow cell in the optical imaging system.

As used herein, a “nucleotide unit” or ‘nucleotide moiety” refers to nucleotides (dATP, dTTP, dGTP, dCTP, or dUTP, e.g.), or analogs thereof, comprising comprises a base, sugar and at least one phosphate group. Nucleotide units can be attached to the multivalent molecules used in the sequencing reactions described herein. In general, all nucleotide units attached to the same multivalent molecule will have the same identity (e.g., all A, all T, all C, or all G), although the skilled artisan will appreciate that there may be situations in which a multivalent molecule comprising nucleotide units of differing identity will be advantageous.

An “optical imaging system” refers to a system configured to illuminate samples using a light source, an illuminator, or the like, and then receive optical signals from the samples, such as flow cells or optical test targets, during imaging sessions. Optical systems of the disclosure can be suitable for fluorescence imaging-based genomics applications. An exemplary optical imaging system includes an objective lens that has a collecting end that is positioned proximate to a sample and configured to receive optical signals therefrom, a tube lens configured to focus the signal from the objective at the detector, a detector that is configured to detect the optical signals (e.g., a camera and/or an eye piece), as well as a light source, such as an excitation light source (e.g., one or more lasers or LED light sources) that can be used to excite the sample during imaging. The optical imaging system may also contain one or more filters (e.g., bandpass filters or acousto-optic tunable filters) that are positioned to filter the light emissions from the sample, such that the sample may be imaged to separately detect different emission spectra (channels, or colors). For example, the optical imaging system may contain one or more excitation light sources and one or more filters, such that a sample may be excited and imaged in each of four different channels corresponding to different labels, each with its own excitation and emission spectrum. The optical imaging system may contain one or more image sensors, e.g., charge-couple device (CCD) image sensors, CMOS image sensors, and the like. In some embodiments, the optical imaging system may only contain a single image sensor. In some embodiments, the optical imaging system may contain multiple image sensors, and each sensor may be used to collect images from a single color channel or shared by multiple different color channels. In some embodiments, the optical imaging system herein may lack any filters that block some portion of the emission light from the one or more samples before the emission light is detected by the image sensor(s). One or more of the components of the optical imaging system may be moveable, such that the system may be adjusted to detect optical signals from a different part (e.g., depth) of the same sample or to calibrate the system. Exemplary optical imaging systems are described in U.S. Pat. No. 9,139,875, the contents of which are incorporated by reference herein.

As used herein, the term “optical signals” includes electromagnetic energy capable of being detected. The term includes light emissions from labeled biological or chemical substances and also includes transmitted light that is refracted or reflected by optical substrates. Optical signals, including excitation radiation that is incident upon the sample and light emissions that are provided by the sample, may have one or more spectral patterns. For example, more than one type of label may be excited in an imaging session. In such cases, the different types of labels may be excited by a common excitation light source or may be excited by different excitation light sources that simultaneously provide incident light. Each type of label may emit optical signals having a spectral pattern that is different from the spectral pattern of other labels. For example, the spectral patterns may have different emission spectra. The light emissions may be filtered to separately detect the optical signals from other emission spectra. As used herein, when the term “different” is used with respect to emission spectra, the emission spectra may have wavelength ranges that at least partially overlap so long as at least a portion of one emission spectrum does not completely overlap the other emission spectrum. Different emission spectra may have other characteristics that do not overlap, such as emission anisotropy or fluorescence lifetime. When the light emissions are filtered, the wavelength ranges of the emission spectra may be narrowed.

As used herein, “refractive index” or “index of refraction” refers to a value calculated from the ratio of the speed of light in a vacuum to that in a second medium of greater density. The refractive index determines how much the path of light is bent, or refracted, when entering a material. Refractive index also varies with wavelength of light. For many materials, the refractive index is known. For example, glass typically has a refractive index of around 1.52, while water has a refractive index of around 1.33. Alternatively, the refractive index of a particular material can be determined by methods known in the art.

As used herein, “light intensity” refers to the number of photons that pass through a given area in a given amount of time, which is separate from the light wavelength or spectrum. With respect to the optical imaging systems described herein, and fluorescent beads employed by said systems, light intensity refers to the light intensity emitted by a fluorescent bead upon excitation, which be determined from images taken by the optical imaging systems using methods known to persons of skill in the art. For example, intensity can be determined from an image using standard image analysis software (ImageJ and the like), and involves selecting the area covered by the fluorescent bead, and determining the pixel values for the pixels in the selected area, which are proportional to the intensity of light emitted by the bead.

“Room temperature” as used herein refers to a temperature range found indoors, usually considered to be between 20° C. and 25° C. Alternatively, room temperature can be between 20° C. and 22° C. As a further alternative, room temperature can be about 20° C., 21° C., 22° C., 23° C., 24° C. or 25° C. Room temperature may not be stable, and fluctuate by 0.5, 1.0, 1.5, 2.0, 2.5, 3.0 or more degrees.

Unless defined otherwise, technical and scientific terms used herein have meanings that are commonly understood by those of ordinary skill in the art unless defined otherwise. Generally, terminologies pertaining to techniques of molecular biology, nucleic acid chemistry, protein chemistry, genetics, microbiology, transgenic cell production, and hybridization described herein are those well-known and commonly used in the art. Techniques and procedures described herein are generally performed according to conventional methods well known in the art and as described in various general and more specific references that are cited and discussed throughout the instant specification. For example, see Sambrook et al., Molecular Cloning: A Laboratory Manual (Third ed., Cold Spring Harbor Laboratory Press, Cold Spring Harbor, N.Y. 2000). See also Ausubel et al., Current Protocols in Molecular Biology, Greene Publishing Associates (1992). The nomenclatures utilized in connection with, and the laboratory procedures and techniques described herein are those well-known and commonly used in the art.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

The headings provided herein are not limitations of the various aspects of the disclosure, which aspects can be understood by reference to the specification as a whole.

### INTRODUCTION

The present disclosure provides solid-state optical test targets useful for evaluating the performance and accuracy of an optical imaging system, for example an optical imaging system used to image flow cells during DNA sequencing analysis. The optical test targets can be configured to simulate the behavior of flow cell devices in an optical imaging system. The disclosure also provides flow cell devices that can be employed with the optical imaging systems disclosed herein, which can be used for performing or facilitating DNA sequencing analysis. The flow cell devices disclosed herein can be used to evaluate the performance of various sequencing systems, including systems whose optical imaging systems have been optimized using the solid-state optical test targets, and can also be used during sequencing to enable image registration of flow cell images acquired with or without the need of fiducial markers external to the flow cell devices. The disclosure also provides sequencing methods for use with the flow cell devices and optical imaging systems disclosed herein.

### Solid-State Optical Test Targets

The present disclosure provides solid-state optical test targets useful for evaluating the performance and accuracy of an optical imaging system, for example a microscope. The optical test targets can be configured to evaluate optical imaging systems, for example optical imaging systems used to image the flow cells used in next generation sequencing. For example, the optical test targets described herein can allow determination of the accuracy of optical alignment, determine autofocus accuracy during imaging; be used to calibrate light sources and cameras; be used when determining an image uniformity correction, or distortion levels, determine contrast across a field of view; determine alignment of the camera; determine the focal distance of the camera, as well as be used to determine field flatness of the light source across the field of view (FOV), focus repeatability, point spread function measurement; and/or modulated transfer function (MTF); determine motion along x, y, and/or z direction; determine spherical aberration and/or chromatic aberration; and/or determine the level of alignment among different color channels. The solid-state optical test targets can be used to evaluate the performance and accuracy of an optical imaging system before any sequencing applications has started, e.g., for calibration of the optical imaging system. Alternatively, the solid-state optical test targets can be used to evaluate the performance and accuracy of an optical imaging system after a sequencing application has started using the sequencing system, e.g., after one or more sequencing cycles have been completed, e.g., for troubling-shooting of the optical imaging system.

The solid-state optical test targets may comprise at least one substrate (e.g., a first substrate) made from a transparent material. The first substrate may have top, bottom and side surfaces. In some embodiments, the top and bottom surfaces are flat. In some embodiments, the first substrate has an even thickness. In some embodiments, the solid-state optical test targets further comprise an opaque coating that forms a micropattern, where the micropattern is configured to include opaque portions and transparent portions. In some embodiments, the optical test targets described herein lack a flow cell and lack a liquid, and are therefore solid-state apparatus.

As disclosed herein, “flatness” herein indicate a leveled quality with raised area or indentations less than a predetermined height or depth from each other, e.g., the height or depth difference between the highest point and the lowest point of a “flat” surface is less than 0.01 um, 0.04 um, 0.06 um, 0.1 um, 0.2 um, 0.5 μm, 0.8 um, 1 μm, 2 um, 5 μm, 8 um, 10 μm, 25 um, 50 μm, 80 um, 100 μm, 250 um, 500 μm, 800 μm, or 1000 um.

In some embodiments, the solid-state optical test targets comprise a first substrate but lack a second substrate. In alternative embodiments, the solid-state optical test targets further comprise a second substrate having top, bottom and side surfaces. In some embodiments, the top surface of the second substrate is flat. In some embodiments, at least a portion of the second substrate comprises an opaque coating that forms a micropattern, the micropattern configured to include opaque portions and transparent portions. In some embodiments, the first substrate is positioned on top of the second substrate. In some embodiments, the first substrate is positioned in direct contact with the micropattern on the second substrate.

In some embodiments, the solid-state optical test target comprises a transparent substrate having a top or bottom surface coated with an opaque layer, wherein the opaque layer forms a micropattern on the substrate. The micropattern can be configured to include opaque portions and transparent portions. In some embodiments, the micropattern comprises an opaque portion with transparent shapes, such as pinholes, lines, or various geometric shapes. The shapes (e.g., pinholes or lines) can be arranged in various pre-determined manners, for example in an array. In some embodiments, each individual transparent portion may include a size and/or shape that is comparable to a cluster or polony immobilized on a flow cell device during next generation sequencing. In some embodiments, each individual transparent portion may include a size that is about the same to or about at least 120%, 110%, 90%, or 80% of a cluster or polony immobilized on a flow cell device during next generation sequencing.

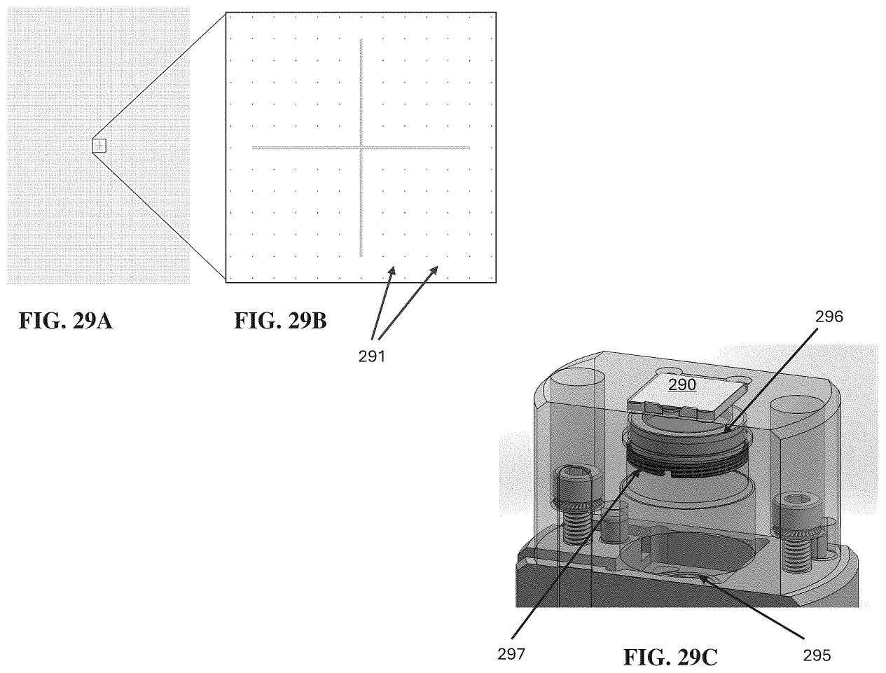

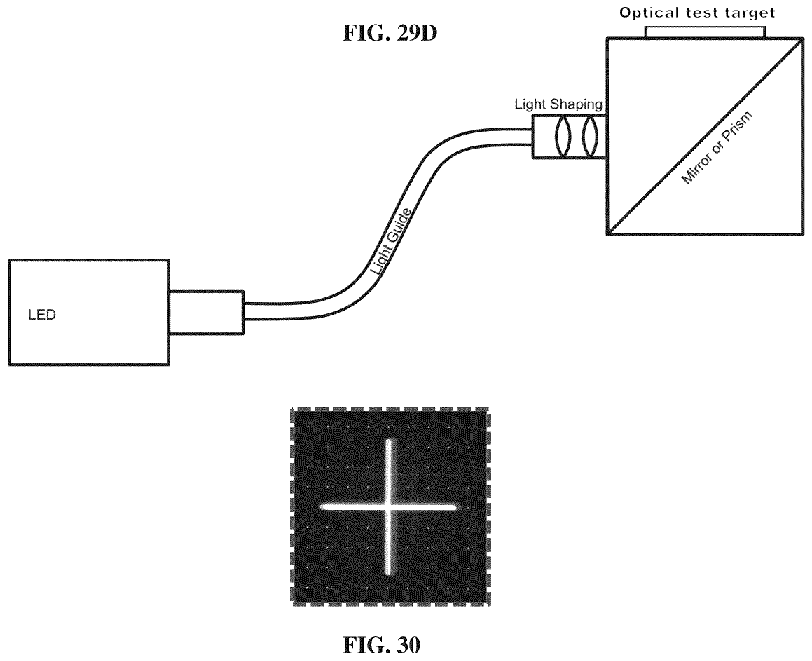

FIGS. 29A-29B show a non-limiting exemplary micropattern of the solid-state optical test targets herein. In this particular embodiment, the exemplary micropattern includes a pinhole array that is distributed across the substrate(s), optionally from a first edge of the substrate to a second edge of the substrate opposite to the first edge. In some embodiments, the micropattern may start from a small distance (e.g., 0.1-8 mm) away from the first edge of the substrate(s) and stop at a small distance (e.g., 0.1-8 mm) to second edge of the substrate. In other words, in some embodiments, the micropattern may cover a portion of the substrate except the area immediate adjacent to the edges of the substrate(s). The portion may be 70%, 80%, 85%, 90%, 95%, 98%, or 99% of the total area of the substrate(s).

In some embodiments, the solid-state optical test targets may include lines or shapes that allow identification of the positioning of the test targets. For example, the solid-state optical test targets may include a cross, a triangle, a diamond, or other various shapes at each corner of the test target to allow positioning of the optical test target in the x-y plane.

FIG. 30 shows an exemplary image of the optical test target in FIGS. 29A-29B detected using the image sensor(s) of the optical imaging system disclosed herein. Non-limiting examples of the image sensor include charge-couple device (CCD) image sensors and CMOS image sensors. In this embodiments, the image shows spatial offsets of different color channels represented by the spatial offset of pinholes and center cross, indicating a need for alignment of different color channels of the optical imaging system.

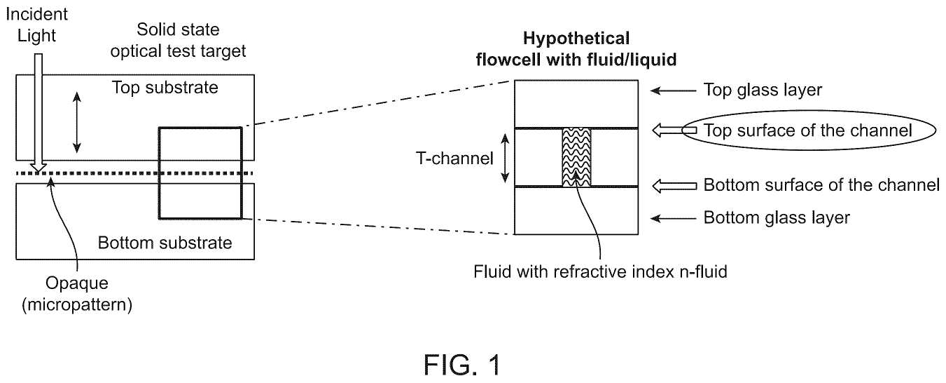

Since the solid-state optical test targets, in some embodiments, lack a flow cell and liquid, the thickness of the first substrate can be adjusted to simulate the effect of a hypothetical flow cell on the transmission of light through the solid-state optical test target when the hypothetical flow cell is located, for example, below the first substrate, or located between the first and second substrates. The adjusted thickness of the first substrate can simulate the collective effects of the first substrate (top substrate) and the hypothetical flow cell containing a fluid/liquid. The hypothetical flow cell comprises a channel having a top surface and bottom surface. The hypothetical flow cell has a height (thickness, or T-channel, see FIG. 1, e.g.). The hypothetical flow cell contains a hypothetical fluid/liquid (e.g., a designated fluid/liquid) with a known refractive index (n-fluid, see FIG. 1). The thickness of the first substrate is configured to permit imaging of the bottom surface of the channel of the hypothetical flow cell. Thus, the solid-state optical test targets described herein are relatively simple to manufacture because they do not require fabrication of a flow cell. Additionally, the solid-state optical test targets enable imaging of the bottom surface of a channel of the hypothetical flow cell by simply adjusting (e.g., increasing) the thickness of the first substrate. The thickness of the first substrate (T-top substrate) that will enable imaging the bottom surface of the hypothetical flow cell can be calculated using the values of the thickness of the hypothetical flow cell channel (T-channel), the refractive index of the hypothetical/designated liquid (n-fluid), and the refractive index of the first substrate (n-top substrate). The general equation is:

[T-top substrate]=([(T-channel]*([(n-fluid]/[n-top substrate])) (Equation 1).

The solid-state optical test target can be assembled by positioning the first substrate (top substrate) on the second substrate (bottom substrate), and the assembled optical test target can be placed in an optical imaging system to evaluate the performance of the optical imaging system. The solid-state optical test target is configurable to evaluate the performance of different optical imaging systems, because the first substrate (top substrate) can be removed and replaced with a different top substrate having a different thickness wherein the replacement top substrate has a thickness that can stimulate the presence of a different hypothetical flow cell having a different channel height. Different top substrates can be manufactured by selecting different values of thicknesses corresponding to different hypothetical flow cell channels (T-channel) and applying the values to the equation described above. Thus, the solid-state optical test targets are modular and adaptable, and can be used to evaluate the performance of different optical imaging systems that are typically used with flow cells for analyzing biological samples, including nucleic acid analysis (e.g., sequencing) and protein analysis, and antibody analysis.

### Solid-State Optical Test Targets for Multi-Surface Imaging

The present disclosure provides a solid-state optical test that may be used for evaluating performance of a sequencing system including the optical imaging system. In some embodiments, the solid-state optical test target disclosed herein may include 1, 2, 3, 4, or more substrates. In some embodiment, each substrate may include a top surface and a bottom surface.

In some embodiments, the solid-state optical test target may be used for evaluating dual surface imaging as disclosed herein. In some embodiments, the solid-state optical test target may be used for evaluating multi-surface imaging which includes 3, 4, or even more surfaces in a flow cell. Although the embodiments herein focuses on the optical test target with a first and a second substrate, in other embodiments, the optical test target can include a first substrate (top), a second substrate (bottom) and a third substrate (mid) that is positioned in between the first and second substrate thereby forming more than one channels, each channel having a top surface and a bottom surface that can be imaged using the optical imaging system described herein. With more than one channel, e.g. 2 channels, displaced from each other at least along the z axis, multiple surfaces (e.g., 3, 4 or more surfaces) may be used for imaging as hypothetical flow cell surfaces that may have sample(s) immobilized thereon.

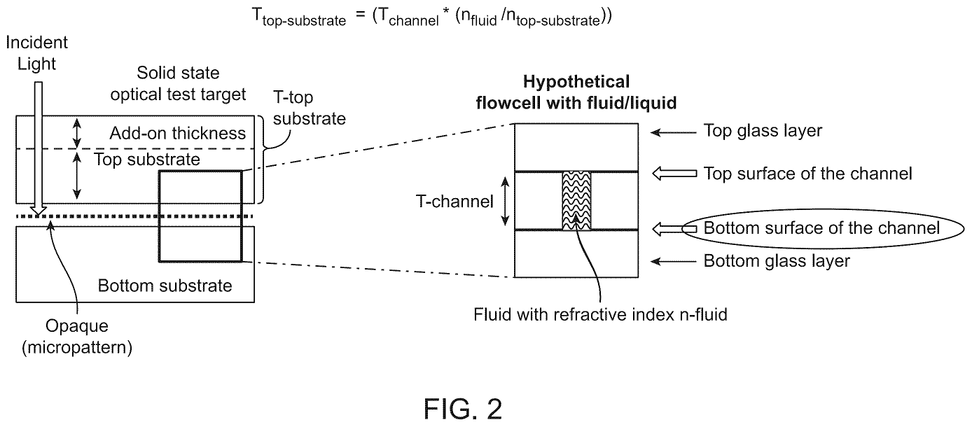

The present disclosure provides a solid-state optical test target (FIG. 2), comprising: (a) a first (e.g., top) substrate (e.g., top substrate) comprising a transparent medium having an even thickness, and having top, bottom and side surfaces, the top and bottom surfaces being flat, and the first substrate having a refractive index of [n-top substrate(1)]; and (b) a second (e.g., bottom) substrate (e.g., bottom substrate) having top, bottom and side surfaces, the top surface being flat. In some embodiments, at least a portion of the top surface of the second substrate comprises an opaque coating that forms a micropattern on a transparent medium, the micropattern configured to include opaque portions and transparent portions. In some embodiments, the transparent medium is glass. In alternative embodiments, transparent medium is plastic. In alternative embodiments, transparent medium comprises a polymer. In some embodiments, the top surface of the second substrate comprises the transparent medium, such that after application of the opaque coating that forms the micropattern, the transparent portions comprise the transparent medium without the opaque coating. In some embodiments, the first substrate is positioned on top of the second substrate, and the first substrate is positioned in direct contact with the micropattern on the second substrate. In some embodiments, the solid-state optical test target lacks a flow cell and lacks a liquid. In some embodiments, the thickness of the first substrate is configured to simulate the presence of a first hypothetical flow cell located between the first and second substrates, wherein the first hypothetical flow cell includes a first channel having a top surface and bottom surface, and the first channel containing a designated first fluid, wherein the first channel has a first designated thickness of [T-channel(1)] and the first designated fluid has a refractive index of [n-fluid(1)]. In some embodiments, the thickness of the first substrate, or a portion thereof, is configured to permit imaging of the bottom surface of the first channel of the first hypothetical flow cell.

In some embodiments, the first substrate comprises transparent glass. In some embodiments, the first substrate comprises transparent plastic.

In some embodiments, the second substrate comprises transparent glass. In some embodiments, the second substrate comprises transparent plastic. In some embodiments, the top, side and/or bottom surfaces of the second substrate are transparent to permit light transmission through the top, side and/or bottom surfaces.

In some embodiments, the bottom surface of the second substrate comprises a reflecting coating. In some embodiments, the bottom surface of the second substrate can be fabricated to have a rough scatter surface.

In some embodiments, the height/thickness of the first substrate [T-top substrate(1)] is related to the refractive index of the first substrate [n-top substrate(1)], the first designated height of the first channel [T-channel(1)] and the refractive index of the first designated fluid [n-fluid(1)], in an equation:

[T-top substrate(1)]=([(T-channel(1)]*([(n-fluid(1)]/[n-top substrate(1)])) (Equation 2)

In some embodiments, the second substrate comprises an opaque coating that forms a micropattern on a top surface. In some embodiments, the thickness of the opaque coating that forms the micropattern on the top surface of the second substrate is between about 50 nm to 150 nm. In some embodiments, the thickness of the opaque coating that forms the micropattern on the top surface of the second substrate is between about 20 nm to 250 nm. In some embodiments, the thickness of the opaque coating that forms the micropattern on the top surface of the second substrate is between about 40 nm to 350 nm. In some embodiments, the thickness of the opaque coating that forms the micropattern on the top surface of the bottom substrate is about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, or about 200 nm. In some embodiments, the thickness of the opaque coating that forms the micropattern on the top surface of the second substrate is about 100 nm. In some embodiments, the micropattern on the top surface of the second substrate can be applied by vapor deposition. In some embodiments, the opaque coating comprises chromium or aluminum.

In some embodiments, leakage of light signal through the opaque coating is below a predetermined threshold. The predetermined threshold may be calculated using background signal (without any sample signal) from flow cell images acquired using the optical imaging system described herein. For example, the opaque coating may allow leakage of light signal through the opaque coating so that the signal level is comparable (e.g., difference is less than ±5%, ±10%, ±20%, ±30%) to the background signal level in flow cell images during sequencing using the optical imaging system herein.

In alternative embodiments, the first substrate comprises an opaque coating that forms a micropattern on a bottom surface. In some embodiments, the thickness of the opaque coating that forms the micropattern on the bottom surface of the first substrate is between about 50 nm to about 150 nm. In some embodiments, the thickness of the opaque coating that forms the micropattern on the top surface of the second substrate is between about 20 nm to about 250 nm. In some embodiments, the thickness of the opaque coating that forms the micropattern on the top surface of the second substrate is between about 40 nm to about 350 nm. In some embodiments, the thickness of the opaque coating that forms the micropattern on the bottom surface of the top substrate is about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, or about 200 nm. In some embodiments, the thickness of the opaque coating that forms the micropattern on the bottom surface of the first substrate is about 100 nm. In some embodiments, the micropattern on the bottom surface of the top substrate can be applied by vapor deposition. In some embodiments, the opaque coating comprises various opaque chemical compounds. In some embodiments, the opaque coating comprises chromium or aluminum.

The transparent portions of the micropattern can comprise repeating shapes arranged in an array. The transparent portions of the micropattern can form a plurality of one type of shape, or a mixture of different types of shapes, wherein the one type of shape and the mixture of different types of shapes are selected from a group consisting of circles, squares and triangles. The transparent portions of the micropattern can form the shape of at least one line, or a plurality of lines arranged in parallel, or a plurality of lines arranged crossing each other, or a plurality of lines arranged in a grid pattern. The transparent portions of the micropattern can form at least one alphanumeric character. The transparent portions of the micropattern can form a plurality of pinholes. The transparent portions of the micropattern can form a plurality of pinholes and a plus sign. The pinholes can fill a full field of view when the solid-state optical test target is positioned in an optical imaging system. In some embodiments, the optical imaging system comprises at least one objective lens, at least one tube lens, and a camera. The transparent portions of the micropattern can form a non-repeating rotationally symmetrical shape, for example including concentric circles (e.g., bulls-eye) or a plus sign (+). In some embodiments, the dimension of any of the transparent portions of the micropattern (e.g., the radius, the length, the size of the transparent portions, etc.) is about 0.1 micron, or about 0.2 microns, or about 0.3 microns, or about 0.4 microns, or about 0.5 microns, or about 0.6 microns, or about 0.7 microns, or about 0.8 microns, or about 0.9 microns, or about 1 micron.

The opaque portions of the micropattern can comprise repeating shapes arranged in an array. The opaque portions of the micropattern can form a plurality of one type of shape, or a mixture of different types of shapes, wherein the one type of shape and the mixture of different types of shapes are selected from a group consisting of circles, squares and triangles. The opaque portions of the micropattern can form the shape of at least one line, or a plurality of lines arranged in parallel, or a plurality of lines arranged crossing each other, or a plurality of lines arranged in a grid pattern. The opaque portions of the micropattern can form at least one alphanumeric character. The opaque portions of the micropattern can form a plurality of pinholes. The opaque portions of the micropattern can form a plurality of pinholes and a plus sign. The pinholes can fill a full field of view when the solid-state optical test target is positioned in an optical imaging system. In some embodiments, the optical imaging system comprises at least one objective lens, at least one tube lens, and a camera. The opaque portions of the micropattern can form a non-repeating rotationally symmetrical shape, for example including concentric circles (e.g., bulls-eye) or a plus sign (+). In some embodiments, the dimension of any of the opaque portions (e.g., a length, a radius, a width, etc.) of the micropattern is about 0.1 micron, or about 0.2 microns, or about 0.3 microns, or about 0.4 microns, or about 0.5 microns, or about 0.6 microns, or about 0.7 microns, or about 0.8 microns, or about 0.9 microns, or about 1 micron. FIG. 29B is an expanded view of a portion of the test target disclosed herein. The opaque portions 291 are approximately opaque squares or rectangles.

In some embodiments, the optical test targets may include a micropattern that comprise features that allow alignment with different levels of precision. In some embodiments, the micropattern and the transparent portions may include one or more first features, e.g., the center cross or plus sign as shown in FIG. 29B, that may be used for alignment at a first precision level. The first feature(s) may be of various geometrical shapes. The first feature(s) may include a combination of lines and shapes. Each first feature may be larger in size than the second feature(s) of the micropattern. In some embodiments, the micropattern may include one or more second feature(s), e.g., the pinholes as shown in FIG. 29B, that may be used for alignment at a second precision level that is greater than the first precision level. In other words, some first features in the micropattern of the test target may be used firstly for coarse alignment, and then second features in the micropattern may be used subsequently for finer alignment. In some embodiments, at least some features of the micropattern may be used for evaluating the performance of the optical imaging system based on the light that is transmitted through the test target.

In some embodiments, the solid-state optical test target can be positioned in an optical imaging system which comprises at least one objective lens, at least one tube lens, and a camera. The optical imaging system can further comprise at least one light source (excitation source; e.g., a laser) and at least one filter. In some embodiments, the optical imaging system can be used for analyzing biological samples, including nucleic acid analysis (e.g., sequencing) and protein analysis, and antibody analysis. In some embodiments, the optical imaging system can be used for DNA sequencing, including sequence-by-synthesis, sequence-by-hybridization or sequence-by-binding procedures. In some embodiments, the optical imaging system can be used for massively parallel sequence-by-synthesis procedures including polony sequencing, pyrosequencing (e.g., from 454 Life Sciences; U.S. Pat. Nos. 7,211,390, 7,244,559 and 7,264,929), chain-terminator sequencing (e.g., from Illumina; U.S. Pat. No. 7,566,537; Bentley 2006 Current Opinion Genetics and Development 16:545-552; and Bentley, et al., 2008 Nature 456:53-59, ion-sensitive sequencing (e.g., from Ion Torrent), probe-anchor ligation sequencing (e.g., Complete Genomics), DNA nanoball sequencing, nanopore DNA sequencing. Examples of single molecule sequencing include Heliscope single molecule sequencing, and single molecule real time (SMRT) sequencing. In some embodiments, the optical imaging system can be used for sequence-by-hybridization including SOLID sequencing (e.g., from Life Technologies; WO 2006/084132), or for sequence-by-binding including Omniome sequencing (e.g., U.S. Pat. No. 10,246,744).

The present disclosure provides methods for evaluating the performance of an optical imaging system, comprising: (a) positioning a solid-state optical test target as shown in FIG. 2 into an optical imaging system which comprises at least one objective lens, at least one tube lens, a camera, at least one light source (excitation source; e.g., laser) and at least one filter; (b) detecting light transmitted through the top (first) substrate; and (c) evaluating the performance of the optical imaging system based on the light that is transmitted through the first substrate.

In some embodiments, the operation(s) of evaluating the performance of the optical imaging system comprises any one or any combination of two or more of: determining the accuracy of the optical alignment; determining the autofocus accuracy; calibrating the light source; calibrating the camera; determining an image uniformity correction; determining distortion levels; determining contrast across a field of view; determining alignment of the camera; determining the focal distance of the camera; determining flat field correction; determining focus repeatability; determining point spread function measurement; and/or determining a modulated transfer function (MTF).

In some embodiments, the operation(s) of evaluating the performance of the optical imaging system comprises any one, or any combination of two or more of: determining optical field flatness of the light source of the optical imaging system at the FOV; determining motion caused by parts of the optical imaging system (e.g., motion caused by vibration) during imaging using the optical system; determining defocus of the optical system; determining spherical aberration and/or chromatic aberration of the optical imaging system; determining field curvature and/or image distortion of the optical imaging system; and determining the signal-to-noise (SNR) and/or contrast-to-noise ratio (CNR) of the optical imaging system.

In some embodiments, the optical field flatness can be evaluated by estimating and/or determining the optical signal intensity variation as a function of position across the FOV at the sample plane. An ideal flat optical field may have identical optical signal intensity and no variation across the FOV. The optical field may be considered as flat, although not perfectly flat, when the optical intensity variation across the FOV is less than a predetermined level. For example, the optical intensity variation may be less than ±25%, ±20%, ±15%, ±10%, ±5%, ±2%, or ±1%.

In some embodiments, the operation(s) of evaluating the performance of the optical imaging system comprises: positioning the solid-state optical test target in an optical imaging system disclosed herein; detecting light transmitted through the first substrate; and evaluating the performance of the optical imaging system based on the light that is transmitted through the optical test target, e.g., the light that is transmitted through the first substrate but not the second substrate. In some embodiments, detecting light transmitted through the optical test target comprises: detecting light transmitted through the optical test target using an image sensor of the optical imaging system; generating images based on the detected light transmitted through the optical test target using a hardware processor of the sequencing system; and analyzing the generated images, either manually or automatically using machine-executable instructions saved on a hardware processor, e.g., of the sequencing system.

In some embodiments, analyzing the generated images, either manually or automatically using machine-executable instructions saved on a hardware processor comprises: analyzing the level of focus of the transparent features in the generated images. The level of focus may be along the z axis orthogonal to the sample stage or sample plane. The generated images can be from different z levels. The operation of analyzing the level of focus of the transparent features in the generated images may comprise: determining an optimal focus position along z axis (e.g., an optimal z level) based on comparing the quality of focus at different z levels. In some embodiments, each quality of focus is based on the focus of at least some of the transparent portions across the FOV at the same z level, e.g., of all pinholes in the images acquired. In some embodiments, the optical focus position along the z axis corresponds to a z level with better focus for the transparent portions across the FOV when compared with the level of focus at other, different, z levels. The operation of analyzing the level of focus of the transparent features in the generated images may further comprise determining field flatness of the optical imaging systems based on the different optical focus positions along the z axis for the transparent portions. Various methods may be used to determine the focus of the transparent portions, including determining the point spread functions of the transparent portions; determining the full width half maximum of Gaussian functions that can fit to different transparent portions; and fitting the optical focus positions along the z axis versus the contrast or sharpness of the transparent portions in the images. The determination of optical field flatness may be based on the operation of analyzing the level of focus of the transparent features in the generated images. Subsequently, different parts of the optical imaging system or the sample may be adjusted or moved in order to improve the field flatness, e.g., at one z location or across multiple different z locations.

In some embodiments, for a single color channel, evaluating the performance of the optical imaging system comprises: determining spherical aberration of the optical imaging system. In some embodiments, images from the single channel may be acquired, and image features of the transparent portions, e.g., full width half maximum of Gaussian fitting to intensities of the transparent portions, may be determined at different x, y, and/or z locations. For example, the full width half maximum (FWHM) of Gaussian fitting of pinholes near the center of the image(s) and at the edges of the images (from a single channel) may be determined. Less difference in FWHM may indicate less spherical aberration in the optical imaging system.

In some embodiments, for a single color channel or multiple color channels, evaluating the performance of the optical imaging system comprises: determining contrast across the FOV. In some embodiments, images from the single channel or multiple color channels may be acquired, and image features of the transparent portions, e.g., intensities of the transparent portions, noise level of the opaque portions, contrast to noise ratio of at least a portion of the images, may be determined at different x, y, and/or z locations. In embodiments where contrast may need to be improved, the light source of the optical system, or other parts of the excitation path or emission path, e.g., the excitation filter, the light transmitter connecting to the light source may be adjusted or moved to improve the contrast in at least some portions of the FOV.

In some embodiments, for a single color channel or multiple color channels, evaluating the performance of the optical imaging system comprises: determining accuracy of autofocus (AF) of the optical imaging system. In some embodiments, images from the single channel or multiple color channels may be acquired, and image features of the transparent portions, e.g., focus changes of the transparent portions at different x and/or y locations along with z location changes can be determined for different z locations. The optimal z location may be determined as where optical focus across the FOV, e.g., average focus of transparent portions is highest among all different z locations. The accuracy of AF of the optical system can be determined based on how far the AF is from the optimal z location.

In some embodiments, for multiple color channels, evaluating the performance of the optical imaging system comprises: determining chromatic aberration of the optical imaging system. In some embodiments, images from multiple color channel may be acquired at a single z level or at different z levels. Image features of the transparent portions, e.g., spatial offset of intensities of the transparent portions may be determined at different x, y, and/or z locations. For example, comparable offsets between corresponding pinholes in images across different color channels may indicate chromatic aberration that is satisfactory for sequencing applications while offsets that changes spatially across the FOV, e.g., larger in the corners than that near the center, may indicate a chromatic aberration level that needs to be improved before performing certain sequencing applications. As another example, the image quality feature may include magnification of the transparent portions. Differences of magnification level across the FOV may be determined based on analysis of the generated images. Image features should not be limited to offsets or magnification of the transparent portions of the generated images. Various other image features that may be obtained from the generated images may also be used, alone or in combination with the offsets and magnification. Improvements of chromatic aberration can include software correction of the chromatic aberration in images acquired during sequencing. Alternatively, the optical imaging system design and its parts may be adjusted or moved to improve the aberration before a sequencing application starts.

In some embodiments, for a single channel or multiple color channels, evaluating the performance of the optical imaging system comprises: determining optical alignment of the optical imaging system. The optical alignment may include alignment of the sample plane, e.g., the plane of the optical test target or the plane of the sample, with the focal plane of the optical system in one or more color channels. In some embodiments, images from a single channel or multiple color channels may be acquired at a single z level or at different z levels. For each z-level, image features of the transparent portions, e.g., focus of the transparent portions, may be determined while adjusting the relative position of the image sensor, e.g., the camera relative to the test target, the objective lens, or other part(s) of the optical imaging system. Improvements of optical alignment can include adjusting or moving the image sensor relative to the optical test target, the objective lens, or other part(s) of the optical image system to improve focus at the z location across the FOV. Such improvement may be repeated when the sample plane is at a different z level.

In some embodiments, for a single channel or multiple color channels, evaluating the performance of the optical imaging system comprises: determining motion of the optical test target caused by parts of the optical imaging system or other parts of the sequencing system. In some embodiments, images from a single channel or multiple color channels may be acquired at a single z level or at different z levels. Image features of the transparent portions, e.g., blurriness, may be determined at different x, y, and/or z locations of the images. Different levels of blurriness of the transparent portions may indicate different levels of motion. The isolation scheme of one or more parts of the optical imaging system may be improved to reduce the blurriness of the images, e.g., the laser, the despeckler, etc., The isolation of the sample, the sample stage, and/or the optical test target may be improved to reduce the blurriness of the images. The isolation of one or more parts of the sequencing system, e.g., the heater or the cooling fan, may be improved to reduce the blurriness of the images.

In some embodiments, for multiple channels, e.g., using an excitation light of the same color, evaluating the performance of the optical imaging system comprises: determining color cross talk between two color channels of the optical imaging system. In some embodiments, images from the two channels may be acquired, with different emission filters, and each emission filter may have a predetermined wavelength range corresponding to the excitation light wavelength. Image features of the transparent portions, e.g., intensities of the same transparent portions may be determined at different x, y, and/or z locations. For example, to determine color cross talk between two green channels, first images of the optical test target may be acquired from a first green channel using a predetermined emission filter that only passes light signal within a second wavelength range but blocks light signal at other wavelengths, the second wavelength range may correspond to emission light of the second green channel. The signal level of the first images, e.g., at different z levels, may indicate the level of color cross-talk between the two green channels. If the signal intensity in first images are comparable to background noise level of the optical test target, the color cross talk may be neglectable, but if the signal intensity in first images are larger than background noise level, e.g., 10× larger, the color cross talk between two color channels may need to be reduced, optionally by correction of color cross talk after acquiring images. Alternatively, color cross talk may be corrected by adjusting parts of the optical imaging system.

In some embodiments, for multiple channels, e.g., using excitation light of the same color, evaluating the performance of the optical imaging system comprises: determining color cross talk between two color channels of the optical imaging system. In some embodiments, color cross-talk can be detected where zero or minimum signal is expected with a predetermined excitation light wavelength range but greater signal is detected in the actual image(s). In some embodiments with a single image sensor, images may be acquired with LED light only in customized wavelength range(s). First images can be acquired with LED light of only a first wavelength range, and second images can be acquired with the LED light of the first wavelength range and a second wavelength range, and third images can be acquired with the LED light of only in the second wavelength range. Image features of the transparent portions in the first, second, and third images, e.g., intensities of the same transparent portions, may be determined at different x, y, and/or z locations. For example, to determine color cross talk between the first and second wavelength ranges, the signal level of the first images, e.g., corresponding pinholes at different z levels, may be compared with the signal level of the second images and/or the third images. If the signal intensities of the same transparent portions the first and second images are comparable, the color cross talk may be negligible. If the signal intensities of the third images are comparable to background noise level of the optical test target, the color cross talk may be negligible, but if the signal intensity in third images are larger than background noise level, e.g., 10× larger, the color cross talk between the first and second wavelength ranges may need to be reduced, optionally by correction of color cross talk after acquiring images. Alternatively, color cross talk may be corrected by adjusting parts of the optical imaging system, e.g., shifting pass band(s) of an emission filter.

In some embodiments, the optical test target may be used for evaluating the optical imaging system that may be used for various applications including different sequencing applications. In some embodiments, the optical test target may be used for evaluating the optical imaging systems of next generation sequencing systems. In some embodiments, the optical test target may be used for evaluating the optical imaging system that may include multiple image sensors that enable simultaneous imaging from different color channels. In some embodiments, the optical test target may be used for evaluating the optical imaging system that may include a single image sensor that enables sequential imaging from different color channels. In some embodiments, the optical test target may be used for evaluating the optical imaging system that include an illuminator that enables illumination of a ultra-wide FOV so that the entire flow cell may be illuminated with homogenous power in less than 1, 2, 4, 6, 8, 10, 12, 16, 18, or 20 FOVs. The homogenous power herein may indicate a variance in power across the FOV for less than 1%, 5%, 10%, 12%, 15%, 20%, 25%, or 30%. In some embodiments, the optical test target may be used for evaluating the optical imaging system that may enable excitation with no greater than 4 color, 2 color, or 1 color to generate images for base calling of the sequencing samples. In some embodiments, the optical test target may be used for evaluating the optical imaging system that may enable excitation with exactly 4 color, 3 color, 2 color, or 1 color to generate images for base calling of the sequencing samples. In some embodiments, the optical test target may be used for evaluating the optical imaging system that may enable imaging and sequencing of 2D or 3D samples, e.g., in situ samples with cells or tissue.

In some embodiments, the sample(s) herein to be sequenced using the sequencing system is immobilized on a flow cell device that remains still on a sample stage. The sample stage may move relative to the objective lens of the optical imaging system or any other part of the optical system to enable focusing of the sample(s), e.g., positioning the sample(s) within the focal plane of the optical imaging system such that the sample plane and the focal plane overlaps at least partly with each other.

In some embodiments, the sample is a 3D sample. In some embodiments the 3D sample includes a cellular sample containing cell(s) and/or tissue(s). In some embodiments, the optical test target can be used to evaluate the sequencing system including the optical imaging system that may advantageously enable sequencing and imaging of target analyte(s) or features while they remain intact inside the cell or tissue. In some embodiments, the cell or tissue and the targets (e.g., target analytes, structure elements, organelles, etc.) therewithin remain intact during evaluation using the optical test target and during sequencing and/or imaging. In some embodiments, the optical test target can be used for evaluating the optical imaging system while the cells or tissue samples are immobilized on the flow cell device for sequencing and/or imaging without modifying the spatial locations of targets within the cells or tissue. In some embodiments, the cells or tissue samples are immobilized on the flow cell device for sequencing or imaging without modifying the spatial relationship of biological analytes (e.g., targets or target analytes) within the cells or tissue. In some embodiments, the cells and/or tissue are immobilized with the morphological features, RNA, mRNA, and protein targets of the samples intact inside the cell(s) or tissue during sequencing and/or imaging. In some embodiments, the spatial locations or relationships of the target analytes or targets remain intact during evaluation of the optical imaging system, during sequencing and/or imaging. In some embodiments, the spatial locations or relationships of the target analytes or targets are not manually reconstructed using artificially added structure or features in the sample(s). For example, the nucleus, cell membrane, mitochondria, and extracellular matrix can retain their relative spatial relationship to each other in the sample(s) during imaging and/or sequencing.

In some embodiments, the sample(s) include biological analytes (e.g., target analyte(s)) that are located inside the sample(s) or on the membrane of the sample(s). In some embodiments, the sample(s) include target analyte(s) that are on the exterior or interior surface of a cell of the sample. In some embodiments, the sample(s) include target analyte(s) that are on the exterior or interior surface of the cell membrane. In some embodiments, the sample(s) include target analyte(s) that are part of the extracellular matrix. In some embodiments, the sample(s) include target analyte(s) that are part of and/or located on one or more organelles within the cell or tissue. In some embodiments, the sample(s) include target analytes that are on or in the glycocalyx or belong to part of the glycocalyx. In some embodiments, the sample(s) include target analyte(s) that are part of and/or located in the cytosol.

In some embodiments, the biological analyte or target analyte(s) comprise at least one polypeptide, lipid, nucleic acid or polysaccharide. In some embodiments, the biological analyte or target analyte(s) comprise at least one polypeptide, enzyme or lipid located anywhere in the sample(s) including without limits the cytoplasm and nucleus. In some embodiments, the biological analyte or target analyte(s) comprise at least one polypeptide, enzyme or lipid located in or on a cellular structure including without limits any cellular membrane, nuclear envelope, nucleus, nucleolus, mitochondria, chloroplast, Golgi apparatus, ribosome, endoplasmic reticulum, microtubules, peroxisome and lysosome.

In some embodiments, the optical test target may be used to evaluate performance of the optical imaging system herein when the sample stage and/or the sample plane is at multiple z-locations. The evaluation of the performance may include one or more operations disclosed herein. The evaluation of the performance of the optical imaging system may be conducted before any sequencing cycles has started yet, e.g., for calibration of the optical imaging system. The evaluation of the performance of the optical imaging system may be conducted before or after the sample(s) has been positioned on the sample stage. In some embodiments, the evaluation of the performance of the optical imaging system may be conducted after one or more sequencing cycles has started, for example, for trouble shooting of the optical imaging system.

Depending on the sample(s) immobilized on the support (e.g., a flow cell device), images may be acquired at single or multiple z locations along an z axis orthogonal to the image plane of the images using the optical system. In particular, for three dimensional samples, e.g., cells, tissues, or other in situ samples, the flow cell images herein can include multiple z-levels (i.e., axial locations) in order to cover the whole sample(s) in 3D. The z axis can extend from the objective lens of the optical system disclosed herein to the support, e.g., flow cell. The z axis can be orthogonal to the image plane of the flow cell images. Each z location of flow cell images may be separated from the adjacent z location(s) for a predetermined distance, for example, for about 0.1 μm to about 15 μm. Each z location of flow cell images may be separated from the adjacent level(s) for 0.5 μm to 10 μm. Each z location of flow cell images may be separated from the adjacent level(s) for 0.2 μm to 2 μm. At each z location, flow cell images can be acquired from one or more sequencing cycles and/or one or more channels. Each flow cell image may include in its field of view at least part of one or more tiles or subtiles of the flow cell.

Solid-State Optical Test Targets with an Exchangeable Top Substrate

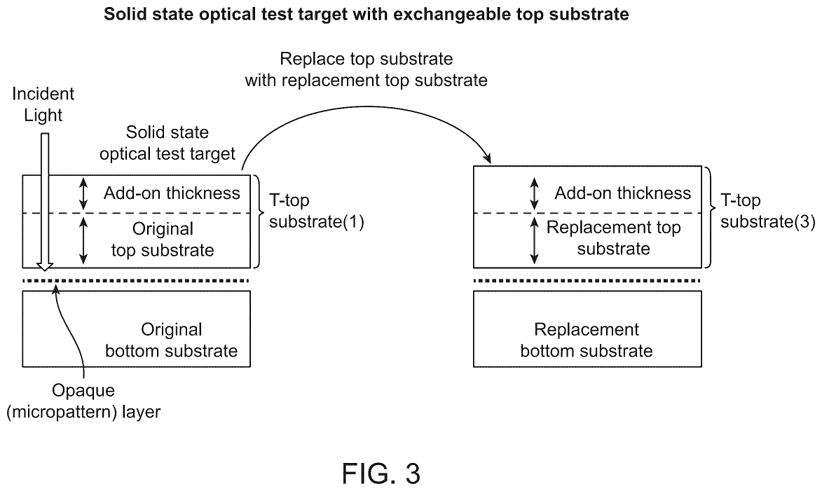

In some embodiments, in the solid-state optical test target, the first (top) substrate is removable from the second (bottom) substrate. In some embodiments, the first substrate is replaced with a third substrate (e.g., replacement top substrate) which comprises a flat transparent medium and having top, bottom and side surfaces, and the third substrate having a refractive index of [n-top substrate(3)] (FIG. 3). In some embodiments, the third substrate is positioned on top of the second substrate, and the third substrate is positioned in direct contact with the micropattern on the second substrate. In some embodiments, the solid-state optical test target which comprises the third (e.g., replacement substrate) and second substrates, lacks a flow cell and lacks a liquid. In some embodiments, the height (thickness) of the third substrate is configured to simulate the presence of a second hypothetical flow cell located between the third and second substrates, wherein the second hypothetical flow cell includes a second channel having a top surface and a bottom surface, and the second channel containing a designated second fluid, wherein the second channel has a second designated thickness of [T-channel(2)] and the second designated fluid has a refractive index of [n-fluid(2)]. In some embodiments, the thickness of the third substrate is configured to permit imaging of the bottom surface of the second channel of the second hypothetical flow cell. See FIG. 3.

In some embodiments, the third substrate comprises transparent glass. In some embodiments, the height/thickness of the third substrate [T-top substrate(3)] is related to the refractive index of the third substrate [n-top substrate(3)], the second designated height of the second channel [T-channel(2)] and the refractive index of the second designated fluid [n-fluid(2)], in an equation:

$$[ T - top substrate ( 3 ) ] = ( [ ( T - channel ( 2 ) ] * ( [ ( n - fluid ( 2 ) ] / [ n - top substrate ( 3 ) ] ) ) . \qquad ( Equation 3 )$$

In some embodiments, the refractive index of the first substrate [n-top substrate(1)] is the same or different from the refractive index of the third substrate [n-top substrate(3)]. In some embodiments, the designated height of the first hypothetical channel [T-channel(1)] is the same or different from the designated height of the second hypothetical channel [T-channel(2)]. In some embodiments, the refractive index of the first designated fluid [n-fluid(1)] is the same or different from the refractive index of the second designated fluid [n-fluid(2)].

In some embodiments, in the solid-state optical test target having the exchangeable top substrate (FIG. 3), the second substrate comprises transparent glass. In some embodiments, the top, side and/or bottom surfaces of the second substrate are transparent to permit light transmission through the top, side and/or bottom surfaces. In some embodiments, the bottom surface of the second substrate comprising a reflecting coating. In some embodiments, the bottom surface of the second substrate can be fabricated to have a rough scatter surface.

In some embodiments, in the solid-state optical test target having the exchangeable top substrate (FIG. 3), at least a portion of the top surface of the second substrate comprises an opaque coating that forms a micropattern, the micropattern configured to include opaque portions and transparent portions, as described supra.

The transparent portions of the micropattern can comprise repeating shapes arranged in an array. The transparent portions of the micropattern can form a plurality of one type of shape, or a mixture of different types of shapes, wherein the one type of shape and the mixture of different types of shapes are selected from a group consisting of circles, squares and triangles. The transparent portions of the micropattern can form the shape of at least one line, or a plurality of lines arranged in parallel, or a plurality of lines arranged crossing each other, or a plurality of lines arranged in a grid pattern. The transparent portions of the micropattern can form at least one alphanumeric character. The transparent portions of the micropattern can form a plurality of pinholes. The transparent portions of the micropattern can form a plurality of pinholes and a plus sign. The pinholes can fill a full field of view when the solid-state optical test target is positioned in an optical imaging system which comprises at least one objective lens, at least one tube lens, and a camera. The transparent portions of the micropattern can form a non-repeating rotationally symmetrical shape, for example including concentric circles (e.g., bulls-eye) or a plus sign (+). In some embodiments, the dimension of any of the transparent portions (e.g., a length, a radius, a width, etc.) of the micropattern is about 0.1 micron, or about 0.2 microns, or about 0.3 microns, or about 0.4 microns, or about 0.5 microns, or about 0.6 microns, or about 0.7 microns, or about 0.8 microns, or about 0.9 microns, or about 1 micron.

The opaque portions of the micropattern can comprise repeating shapes arranged in an array. The opaque portions of the micropattern can form a plurality of one type of shape, or a mixture of different types of shapes, wherein the one type of shape and the mixture of different types of shapes are selected from a group consisting of circles, squares and triangles. The opaque portions of the micropattern can form the shape of at least one line, or a plurality of lines arranged in parallel, or a plurality of lines arranged crossing each other, or a plurality of lines arranged in a grid pattern. The opaque portions of the micropattern can form at least one alphanumeric character. The opaque portions of the micropattern can form a plurality of pinholes. The opaque portions of the micropattern can form a plurality of pinholes and a plus sign. The pinholes can fill a full field of view when the solid-state optical test target is positioned in an optical imaging system which comprises at least one objective lens, at least one tube lens, and a camera. The opaque portions of the micropattern can form a non-repeating rotationally symmetrical shape, for example including concentric circles (e.g., bulls-eye) or a plus sign (+). In some embodiments, the dimension of any of the opaque portions (e.g., a length, a radius, a width, etc.) of the micropattern is about 0.1 micron, or about 0.2 microns, or about 0.3 microns, or about 0.4 microns, or about 0.5 microns, or about 0.6 microns, or about 0.7 microns, or about 0.8 microns, or about 0.9 microns, or about 1 micron.

In some embodiments, in the solid-state optical test target having the exchangeable top substrate (FIG. 3), the solid-state optical test target can be positioned in an optical imaging system which comprises at least one objective lens, at least one tube lens, and a camera. The optical imaging system can further comprise at least one light source (excitation source; e.g., laser) and at least one filter.

The present disclosure provides methods for evaluating the performance of an optical imaging system, comprising: (a) positioning the solid-state optical test target having the exchangeable top substrate (e.g., the third substrate) as shown in FIG. 3 into an optical imaging system which comprises at least one objective lens, at least one tube lens, a camera, at least one light source (excitation source; e.g., laser) and at least one filter; (b) detecting light transmitted through the third substrate; and (c) evaluating the performance of the optical imaging system based on the light that is transmitted through the third substrate.

In some embodiments, the operation(s) of evaluating the performance of the optical imaging system comprises any one or any combination of two or more of: determining the accuracy of the optical alignment; determining the autofocus accuracy; calibrating the light source; calibrating the camera; determining an image uniformity correction; determining distortion levels; determining contrast across a field of view; determining alignment of the camera; determining the focal distance of the camera; determining flat field correction; determining focus repeatability; determining point spread function measurement; and/or determining a modulated transfer function (MTF).

In some embodiments, the operation(s) of evaluating the performance of the optical imaging system comprises any one or any combination of two or more of: determining optical field flatness of the light source of the optical imaging system at the FOV; determining motion caused by parts of the optical system (e.g., motion caused by vibration) during imaging using the optical system; determining defocus of the optical system; determining spherical aberration and/or chromatic aberration of the optical imaging system; determining field curvature and/or image distortion of the optical imaging system; and determining the SNR and/or contrast-to-noise ratio (CNR) of the optical imaging system.

Solid-State Optical Test Targets with at Least One Fluorescent Dye Layer

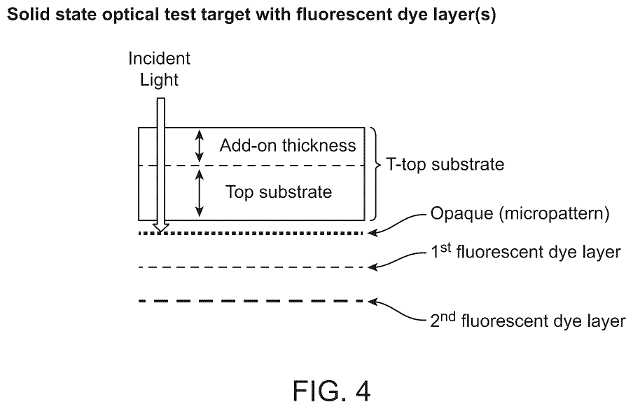

The present disclosure provides a solid-state optical test target comprising a first substrate but lacking a second substrate. The bottom surface of the first substrate can have an opaque coating, and a coating having at least one type of fluorescent dye (FIG. 4). In some embodiments, the bottom surface of the first substrate comprises an opaque layer to form a micropattern, wherein the micropattern is configured to include opaque portions and transparent portions. The opaque layer can be coated with at least a first coating which comprises one type of a fluorescent dye, or a mixture of two or more different types of fluorescent dyes. In some embodiments, the first coating of fluorescent dyes can have a second coating of fluorescent dyes, wherein the second coating comprises one type of a fluorescent dye, or a mixture of two or more different types of fluorescent dyes. Suitable fluorescent dyes will be known to the skilled artisan, and include, inter alia, cyanins such as Cy3, or Cy5, etc., fluoresceins, coumarins, rhodamines, etc. or other dyes disclosed herein.

In some embodiments, the fluorescent solid-state optical test target (FIG. 4) comprises: (a) a substrate comprising a transparent medium having an even thickness, and comprising top, bottom and side surfaces, the top and bottom surfaces being flat, wherein at least a portion of the bottom surface of the substrate comprises an opaque coating that forms a micropattern, wherein the micropattern is configured to include opaque portions and transparent portions, and the substrate has a refractive index of [n-top substrate(1)]; and (b) at least one layer of fluorescent dyes layered (or coated) on the bottom surface of the substrate on the opaque coating, wherein (i) the fluorescent solid-state optical test target lacks a flow cell and lacks a liquid, (ii) the thickness of the substrate is configured to simulate the presence of a first hypothetical flow cell located between a first and second substrate, wherein the first hypothetical flow cell includes a first channel having a top surface and bottom surface, and containing a designated first fluid, wherein the first channel has a first designated thickness of [T-channel(1)] and the first designated fluid has a refractive index of [n-fluid(1)], and (iii) the thickness of the substrate is configured to permit imaging of the bottom surface of the first channel of the first hypothetical flow cell. In some embodiments, at least one layer of fluorescent dyes (e.g., first layer of fluorescent dye) comprises one type of a fluorescent dye, or a mixture of two or more different types of fluorescent dyes. In some embodiments, the fluorescent solid-state optical test target further comprise a second layer of fluorescent dyes layered on the first layer of fluorescent dyes.

In some embodiments, the second layer of fluorescent dyes comprises one type of a fluorescent dye, or a mixture of two or more different types of fluorescent dyes. In some embodiments, the fluorescent spectrum of the dye in the first coating does not substantially overlap with the fluorescence spectrum of the dye in the second coating. When the fluorescent dye coating (e.g., first layer and/or second layer) comprises a mixture of two or more different types of dyes, the fluorescent spectrum of the dyes in the mixture do not substantially overlap each other.

In some embodiments, the solid-state optical test target has only a top substrate, wherein the bottom surface of the top substrate has multiple coatings, including an opaque layer and at least one fluorescent dye layer (FIG. 4), the height/thickness of the first substrate [T-top substrate(1)] is related to the refractive index of the first substrate [n-top substrate(1)], the first designated height of the first channel [T-channel(1)] and the refractive index of the first designated fluid [n-fluid(1)], in an equation:

$$[ T - top substrate ( 1 ) ] = ( [ ( T - channel ( 1 ) ] * ( [ ( n - fluid ( 1 ) ] / [ n - top substrate ( 1 ) ] ) ) . \qquad ( Equation 2 )$$

In some embodiments, for example in the solid-state optical test target shown in FIG. 4, the thickness of the opaque coating that forms the micropattern is about 100 nm. In some embodiments, the thickness of the opaque coating is between about 50 nm to about 150 nm. In some embodiments, the thickness of the opaque coating that forms the micropattern on the top surface of the second substrate is between about 20 nm to about 250 nm. In some embodiments, the thickness of the opaque coating that forms the micropattern on the top surface of the second substrate is between about 40 nm to 350 nm. In some embodiments, the thickness of the opaque coating is about 50 nm, about 60 nm, about 70 nm, about 80 nm, about 90 nm, about 100 nm, about 110 nm, about 120 nm, about 130 nm, about 140 nm or about 150 nm. In some embodiments, the micropattern can be applied by vapor deposition. In some embodiments, the opaque coating comprises chromium or aluminum.

The transparent portions of the micropattern can comprise repeating shapes arranged in an array. The transparent portions of the micropattern can form a plurality of one type of shape, or a mixture of different types of shapes, wherein the one type of shape and the mixture of different types of shapes are selected from a group consisting of circles, squares and triangles. The transparent portions of the micropattern can form the shape of at least one line, or a plurality of lines arranged in parallel, or a plurality of lines arranged crossing each other, or a plurality of lines arranged in a grid pattern. The transparent portions of the micropattern can form at least one alphanumeric character. The transparent portions of the micropattern can form a plurality of pinholes. The transparent portions of the micropattern can form a plurality of pinholes and a plus sign. The pinholes can fill a full field of view when the solid-state optical test target is positioned in an optical imaging system which comprises at least one objective lens, at least one tube lens, and a camera. The transparent portions of the micropattern can form a non-repeating rotationally symmetrical shape, for example including concentric circles (e.g., bulls-eye) or a plus sign (+). In some embodiments, the dimension of any of the transparent portions of the micropattern is about 0.1 micron, or about 0.2 microns, or about 0.3 microns, or about 0.4 microns, or about 0.5 microns, or about 0.6 microns, or about 0.7 microns, or about 0.8 microns, or about 0.9 microns, or about 1 micron.

The opaque portions of the micropattern can comprise repeating shapes arranged in an array. The opaque portions of the micropattern can form a plurality of one type of shape, or a mixture of different types of shapes, wherein the one type of shape and the mixture of different types of shapes are selected from a group consisting of circles, squares and triangles. The opaque portions of the micropattern can form the shape of at least one line, or a plurality of lines arranged in parallel, or a plurality of lines arranged crossing each other, or a plurality of lines arranged in a grid pattern. The opaque portions of the micropattern can form at least one alphanumeric character. The opaque portions of the micropattern can form a plurality of pinholes. The opaque portions of the micropattern can form a plurality of pinholes and a plus sign. The pinholes can fill a full field of view when the solid-state optical test target is positioned in an optical imaging system which comprises at least one objective lens, at least one tube lens, and a camera. The opaque portions of the micropattern can form a non-repeating rotationally symmetrical shape, for example including concentric circles (e.g., bulls-eye) or a plus sign (+). In some embodiments, the dimension of any of the opaque portions of the micropattern is about 0.1 micron, or about 0.2 microns, or about 0.3 microns, or about 0.4 microns, or about 0.5 microns, or about 0.6 microns, or about 0.7 microns, or about 0.8 microns, or about 0.9 microns, or about 1 micron.

In some embodiments, for example in the solid-state optical test target shown in FIG. 4, the first layer of fluorescent dyes comprises a fluorescent dye having an excitation spectrum of 520-540 nm which can produce a green band emission of 530-630 nm. In some embodiments, the first layer of fluorescent dyes comprises a mixture of two fluorescent dyes, the first fluorescent dye having an excitation spectrum of 520-540 nm which can produce a green band emission of 530-630 nm, and the second fluorescent dye having an excitation spectrum of 630-650 nm which can produce a red band emission of 630-690 nm. In some embodiments, the first layer of fluorescent dyes comprises a first fluorescent dye having an excitation spectrum of 520-540 nm which can produce a green band emission of 530-630 nm, and the second layer of fluorescent dyes comprises a second fluorescent dye having an excitation spectrum of 630-650 nm which can produce a red band emission of 630-690 nm, as shown in Table 1.

TABLE 1
Emission Wavelength

| | G1 | G2 | R1 | R2 |
|---|---|---|---|---|
| | band (nm) | band (nm) | band (nm) | band (nm) |
| Band | 530-580 | 580-630 | 630-690 | 690-750 |
| wavelength | | | | |

In some embodiments, for example in the solid-state optical test target shown in FIG. 4, the first layer of fluorescent dyes comprises a fluorescent dye having an excitation spectrum of 500-580 nm which can produce a green band emission of 520-660 nm. In some embodiments, the first layer of fluorescent dyes comprises a mixture of two fluorescent dyes, the first fluorescent dye having an excitation spectrum of 500-580 nm which can produce a green band emission of 520-660 nm, and the second fluorescent dye having an excitation spectrum of 620-680 nm which can produce a red band emission of 620-710 nm. In some embodiments, the first layer of fluorescent dyes comprises a first fluorescent dye having an excitation spectrum of 500-580 nm which can produce a green band emission of 520-660 nm, and the second layer of fluorescent dyes comprises a second fluorescent dye having an excitation spectrum of 620-680 nm which can produce a red band emission of 620-710 nm.

In some embodiments, the solid-state optical test target shown in FIG. 4 can be positioned in an optical imaging system which comprises at least one objective lens, at least one tube lens, and a camera. The optical imaging system can further comprise at least one light source (excitation source; e.g., laser, and/or an LED excitation light) and at least one filter.

The present disclosure provides methods for evaluating the performance of an optical imaging system, comprising: (a) positioning the solid-state optical test target shown in FIG. 4 into an optical imaging system which comprises at least one objective lens, at least one tube lens, a camera, at least one light source (excitation source; e.g., laser and/or an LED excitation light) and at least one filter; (b) detecting light transmitted through the substrate which comprises on its bottom surface an opaque coating and at least one fluorescent dye coating; and (c) evaluating the performance of the optical imaging system based on the light that is transmitted through the substrate.

In some embodiments, the operation(s) of evaluating the performance of the optical imaging system comprises any one or any combination of two or more of: determining the accuracy of the optical alignment; determining the autofocus accuracy; calibrating the light source; calibrating the camera; determining an image uniformity correction; determining distortion levels; determining contrast across a field of view; determining alignment of the camera; determining the focal distance of the camera; determining flat field correction; determining focus repeatability; determining point spread function measurement; and/or determining a modulated transfer function (MTF).

In some embodiments, the operation(s) of evaluating the performance of the optical imaging system comprises any one or any combination of two or more of: determining optical field flatness of the light source of the optical imaging system at the FOV; determining motion caused by parts of the optical system (e.g., motion caused by vibration) during imaging using the optical system; determining defocus of the optical system; determining spherical aberration and/or chromatic aberration of the optical imaging system; determining field curvature and/or image distortion of the optical imaging system; and determining the SNR and/or contrast-to-noise ratio (CNR) of the optical imaging system.

### Adaptive Solid-State Optical Test Targets

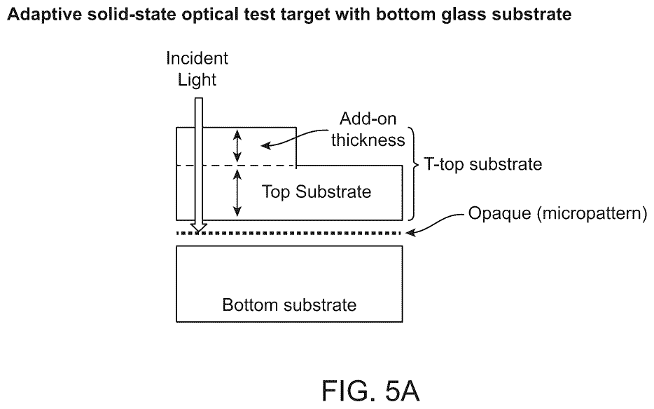

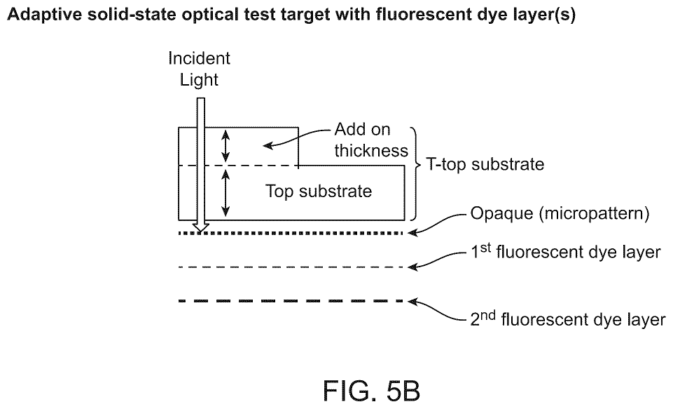

The present disclosure provides an adaptive solid-state optical test target comprising at least a first substrate having at least two regions with different thicknesses (FIGS. 5A and 5B). At least one of the regions has a thickness that is configured to simulate the presence of a first hypothetical flow cell located between the first and second substrates.

Adaptive Solid-State Optical Test Targets with a Transparent Bottom Substrate

In some embodiments, the adaptive solid-state optical test targets comprise first and second substrates, wherein the first substrate is positioned on the second substrate (FIG. 5A), and wherein the top surface of the second substrate comprises an opaque layer to form a micropattern, wherein the micropattern is configured to include opaque portions and transparent portions.

In some embodiments, the adaptive solid-state optical test target (FIG. 5A) comprises: (a) a first substrate (e.g., a top substrate) comprising a transparent medium with top, bottom and side surfaces, the bottom surface being flat, the first substrate having at least two regions with different thicknesses, wherein the first region is flat and has a first thickness and the second region is flat and has a second thickness, and the first substrate having a refractive index of [n-top substrate(1)]; and (b) a second substrate (e.g., bottom substrate) having top, bottom and side surfaces, the top surface being flat, wherein (i) at least a portion of the top surface of the second substrate comprises an opaque coating that forms a micropattern, the micropattern configured to include opaque portions and transparent portions. In some embodiments, the transparent medium is glass. In alternative embodiments, transparent medium is plastic. In alternative embodiments, transparent medium comprises a polymer. In some embodiments, the top surface of the second substrate comprises the transparent medium, such that after application of the opaque coating that forms the micropattern, the transparent portions comprise the transparent medium without the opaque coating. In some embodiments, the first substrate is positioned on top of the second substrate, and the first substrate is positioned in direct contact with the micropattern on the second substrate. In some embodiments, the adaptive solid-state optical test target lacks a flow cell and lacks a liquid. In some embodiments, the thickness of the first region of the first substrate is configured to simulate the presence of a first hypothetical flow cell located between the first and second substrates, wherein the first hypothetical flow cell includes a first channel having a top surface and a bottom surface, and the first channel containing a designated first fluid, wherein the first channel has a first designated thickness of [T-channel(1)] and the first designated fluid has a refractive index of [n-fluid(1)]. In some embodiments, the thickness of the first region of the first substrate is configured to permit imaging of the bottom surface of the first channel of the first hypothetical flow cell. In some embodiments, the thickness of the second region of the first substrate is configured to permit imaging of the top surface of the first channel of the first hypothetical flow cell (FIG. 5A). In some embodiments, the first substrate comprises transparent glass. In some embodiments, the second substrate comprises transparent glass. In some embodiments, the top, side and/or bottom surfaces of the second substrate are transparent to permit light transmission through the top, side and/or bottom surfaces. In some embodiments, the bottom surface of the second substrate comprising a reflecting coating. In some embodiments, the bottom surface of the second substrate can be fabricated to have a rough scatter surface.

In some embodiments, the height/thickness of the first region (FIG. 5A) of the first substrate [T-top substrate(1)] is related to the refractive index of the first substrate [n-top substrate(1)], the first designated height of the first channel [T-channel(1)] and the refractive index of the first designated fluid [n-fluid(1)], in an equation:

$$[ T - top substrate ( 1 ) ] = ( [ ( T - channel ( 1 ) ] * ( [ ( n - fluid ( 1 ) ] / [ n - top substrate ( 1 ) ] ) ) . \qquad ( Equation 2 )$$

In some embodiments, the thickness of the opaque coating that forms the micropattern on the top surface of the second substrate is about 100 nm. In some embodiments, the thickness of the opaque coating is between about 50 nm to about 150 nm. In some embodiments, the thickness of the opaque coating that forms the micropattern on the top surface of the second substrate is between about 20 nm to about 250 nm. In some embodiments, the thickness of the opaque coating that forms the micropattern on the top surface of the second substrate is between about 40 nm to about 350 nm. In some embodiments, the thickness of the opaque coating is about 50 nm, about 60 nm, about 70 nm, about 80 nm, about 90 nm, about 100 nm, about 110 nm, about 120 nm, about 130 nm, about 140 nm or about 150 nm. In some embodiments, the micropattern on the top surface of the second substrate can be applied by vapor deposition. In some embodiments, the opaque coating comprises chromium or aluminum.

The transparent portions of the micropattern can comprise repeating shapes arranged in an array. The transparent portions of the micropattern can form a plurality of one type of shape, or a mixture of different types of shapes, wherein the one type of shape and the mixture of different types of shapes are selected from a group consisting of circles, squares and triangles. The transparent portions of the micropattern can form the shape of at least one line, or a plurality of lines arranged in parallel, or a plurality of lines arranged crossing each other, or a plurality of lines arranged in a grid pattern. The transparent portions of the micropattern can form at least one alphanumeric character. The transparent portions of the micropattern can form a plurality of pinholes. The transparent portions of the micropattern can form a plurality of pinholes and a plus sign. The pinholes can fill a full field of view when the solid-state optical test target is positioned in an optical imaging system which comprises at least one objective lens, at least one tube lens, and a camera. The transparent portions of the micropattern can form a non-repeating rotationally symmetrical shape, for example including concentric circles (e.g., bulls-eye) or a plus sign (+). In some embodiments, the dimension of any of the transparent portions of the micropattern is about 0.1 micron, or about 0.2 microns, or about 0.3 microns, or about 0.4 microns, or about 0.5 microns, or about 0.6 microns, or about 0.7 microns, or about 0.8 microns, or about 0.9 microns, or about 1 micron.

The opaque portions of the micropattern can comprise repeating shapes arranged in an array. The opaque portions of the micropattern can form a plurality of one type of shape, or a mixture of different types of shapes, wherein the one type of shape and the mixture of different types of shapes are selected from a group consisting of circles, squares and triangles. The opaque portions of the micropattern can form the shape of at least one line, or a plurality of lines arranged in parallel, or a plurality of lines arranged crossing each other, or a plurality of lines arranged in a grid pattern. The opaque portions of the micropattern can form at least one alphanumeric character. The opaque portions of the micropattern can form a plurality of pinholes. The opaque portions of the micropattern can form a plurality of pinholes and a plus sign. The pinholes can fill a full field of view when the solid-state optical test target is positioned in an optical imaging system which comprises at least one objective lens, at least one tube lens, and a camera. The opaque portions of the micropattern can form a non-repeating rotationally symmetrical shape, for example including concentric circles (e.g., bulls-eye) or a plus sign (+). In some embodiments, the dimension of any of the opaque portions (e.g., a length, a radius, a width, etc.) of the micropattern is about 0.1 micron, or about 0.2 microns, or about 0.3 microns, or about 0.4 microns, or about 0.5 microns, or about 0.6 microns, or about 0.7 microns, or about 0.8 microns, or about 0.9 microns, or about 1 micron.

In some embodiments, the adaptive solid-state optical test target shown in FIG. 5A is positioned in an optical imaging system which comprises at least one objective lens, at least one tube lens, and a camera. In some embodiments, the optical imaging system further comprises at least one light source (excitation source; e.g., laser and/or an LED excitation light) and at least one filter.

The present disclosure provides methods for evaluating the performance of an optical imaging system, comprising: (a) positioning the solid-state optical test target shown in FIG. 5A into an optical imaging system which comprises at least one objective lens, at least one tube lens, a camera, at least one light source (excitation source; e.g., laser) and at least one filter; (b) detecting light transmitted through a first region of a first substrate of solid-state optical test target; and (c) evaluating the performance of the optical imaging system based on the light that is transmitted through the first region of the first substrate. In some embodiments, the method comprises: (a) positioning the solid-state optical test target shown in FIG. 5A into an optical imaging system which comprises at least one objective lens, at least one tube lens, a camera, at least one light source (excitation source; e.g., laser and/or an LED excitation light) and at least one filter; (b) detecting light transmitted through a first region of a first substrate of the solid-state optical test target; (c) detecting light transmitted through a second region of the first substrate and (d) evaluating the performance of the optical imaging system based on the light that is transmitted through the first and second regions of the first substrate.

In some embodiments, the operation(s) of evaluating the performance of the optical imaging system comprises any one or any combination of two or more of: determining the accuracy of the optical alignment; determining the autofocus accuracy; calibrating the light source; calibrating the camera; determining an image uniformity correction; determining distortion levels; determining contrast across a field of view; determining alignment of the camera; determining the focal distance of the camera; determining flat field correction; determining focus repeatability; determining point spread function measurement; and/or determining a modulated transfer function (MTF).

In some embodiments, the operation(s) of evaluating the performance of the optical imaging system comprises any one or any combination of two or more of: determining optical field flatness of the light source of the optical imaging system at the FOV; determining motion caused by parts of the optical system (e.g., motion caused by vibration) during imaging using the optical system; determining defocus of the optical system; determining spherical aberration and/or chromatic aberration of the optical imaging system; determining field curvature and/or image distortion of the optical imaging system; and determining the SNR and/or contrast-to-noise ratio (CNR) of the optical imaging system.

Adaptive Solid-State Optical Test Targets with Fluorescent Dye Layer(s)

In some embodiments, the adaptive solid-state optical test targets comprises a first substrate but lacks a second substrate, wherein a bottom surface of the first substrate has an opaque coating, and a coating having at least one type of fluorescent dye (FIG. 5B). The bottom surface of the first substrate comprises an opaque layer to form a micropattern, wherein the micropattern is configured to include opaque portions and transparent portions. The opaque layer can be coated with at least a first coating which comprises one type of a fluorescent dye, or a mixture of two or more different types of fluorescent dyes. In some embodiments, the first coating of fluorescent dyes can have a second coating of fluorescent dyes, wherein the second coating comprises one type of a fluorescent dye, or a mixture of two or more different types of fluorescent dyes.

In some embodiments, the adaptive solid-state optical test target (FIG. 5B) comprises: (a) a substrate (e.g., top substrate) comprising a transparent medium with top, bottom and side surfaces, the bottom surface being flat, the substrate having at least two regions with different thicknesses, wherein the first region is flat and has a first thickness and the second region is flat and has a second thickness, and the substrate having a refractive index of [n-top substrate(1)]; and (b) at least one layer of fluorescent dyes layered on the bottom surface of the substrate wherein the fluorescent dye layer is layered on the opaque coating. In some embodiments, at least a portion of the bottom surface of the substrate comprises an opaque coating that forms a micropattern, the micropattern configured to include opaque portions and transparent portions. In some embodiments, the transparent medium is glass. In alternative embodiments, transparent medium is plastic. In alternative embodiments, the transparent medium comprises a polymer. In some embodiments, the bottom surface of the substrate comprises the transparent medium, such that after application of the opaque coating that forms the micropattern, the transparent portions comprise the transparent medium without the opaque coating. In some embodiments, the adaptive solid-state optical test target lacks a flow cell and lacks a liquid. In some embodiments, the thickness of the first region of the substrate is configured to simulate the presence of a first hypothetical flow cell located between a first and second substrate, wherein the first hypothetical flow cell includes a first channel having a top surface and a bottom surface, and the first channel containing a designated first fluid, wherein the first channel has a first designated thickness of [T-channel(1)] and the first designated fluid has a refractive index of [n-fluid(1)]. In some embodiments, the thickness of the first region of the substrate is configured to permit imaging of the bottom surface of the first channel of the first hypothetical flow cell. In some embodiments, the thickness of the second region of the substrate is configured to permit imaging of the top surface of the first channel of the first hypothetical flow cell. In some embodiments, the substrate comprises transparent glass.

In some embodiments, in the adaptive solid-state optical test target (FIG. 5B), the height/thickness of the first region of the substrate [T-top substrate(1)] is related to the refractive index of the substrate [n-top substrate(1)], the first designated height of the first channel [T-channel(1)] and the refractive index of the first designated fluid [n-fluid(1)], in an equation

$$[ T - top substrate ( 1 ) ] = ( [ ( T - channel ( 1 ) ] * ( [ ( n - fluid ( 1 ) ] / [ n - top substrate ( 1 ) ] ) ) . \qquad ( Equation 2 )$$

In some embodiments, the thickness of the opaque coating that forms the micropattern on the bottom surface of the first substrate (FIG. 5B) is about 100 nm. In some embodiments, the thickness of the opaque coating is between about 50 nm to about 150 nm. In some embodiments, the thickness of the opaque coating that forms the micropattern on the top surface of the second substrate is between about 20 nm to about 250 nm. In some embodiments, the thickness of the opaque coating that forms the micropattern on the top surface of the second substrate is between about 40 nm to about 350 nm. In some embodiments, the thickness of the opaque coating is about 50 nm, about 60 nm, about 70 nm, about 80 nm, about 90 nm, about 100 nm, about 110 nm, about 120 nm, about 130 nm, about 140 nm or about 140 nm. In some embodiments, the micropattern on the bottom surface of the first substrate can be applied by vapor deposition. In some embodiments, the opaque coating comprises chromium or aluminum.

The transparent portions of the micropattern can comprise repeating shapes arranged in an array. The transparent portions of the micropattern can form a plurality of one type of shape, or a mixture of different types of shapes, wherein the one type of shape and the mixture of different types of shapes are selected from a group consisting of circles, squares and triangles. The transparent portions of the micropattern can form the shape of at least one line, or a plurality of lines arranged in parallel, or a plurality of lines arranged crossing each other, or a plurality of lines arranged in a grid pattern. The transparent portions of the micropattern can form at least one alphanumeric character. The transparent portions of the micropattern can form a plurality of pinholes. The transparent portions of the micropattern can form a plurality of pinholes and a plus sign. The pinholes can fill a full field of view when the solid-state optical test target is positioned in an optical imaging system which comprises at least one objective lens, at least one tube lens, and a camera. The transparent portions of the micropattern can form a non-repeating rotationally symmetrical shape, for example including concentric circles (e.g., bulls-eye) or a plus sign (+). In some embodiments, the dimension of any of the transparent portions of the micropattern is about 0.1 micron, or about 0.2 microns, or about 0.3 microns, or about 0.4 microns, or about 0.5 microns, or about 0.6 microns, or about 0.7 microns, or about 0.8 microns, or about 0.9 microns, or about 1 micron.

The opaque portions of the micropattern can comprise repeating shapes arranged in an array. The opaque portions of the micropattern can form a plurality of one type of shape, or a mixture of different types of shapes, wherein the one type of shape and the mixture of different types of shapes are selected from a group consisting of circles, squares and triangles. The opaque portions of the micropattern can form the shape of at least one line, or a plurality of lines arranged in parallel, or a plurality of lines arranged crossing each other, or a plurality of lines arranged in a grid pattern. The opaque portions of the micropattern can form at least one alphanumeric character. The opaque portions of the micropattern can form a plurality of pinholes. The opaque portions of the micropattern can form a plurality of pinholes and a plus sign. The pinholes can fill a full field of view when the solid-state optical test target is positioned in an optical imaging system which comprises at least one objective lens, at least one tube lens, and a camera. The opaque portions of the micropattern can form a non-repeating rotationally symmetrical shape, for example including concentric circles (e.g., bulls-eye) or a plus sign (+). In some embodiments, the dimension of any of the opaque portions (e.g., a length, a radius, a width, etc.) of the micropattern is about 0.1 micron, or about 0.2 microns, or about 0.3 microns, or about 0.4 microns, or about 0.5 microns, or about 0.6 microns, or about 0.7 microns, or about 0.8 microns, or about 0.9 microns, or about 1 micron.

In some embodiments, at least one layer of fluorescent dyes (e.g., first layer of fluorescent dye) comprises one type of a fluorescent dye, or a mixture of two or more different types of fluorescent dyes. In some embodiments, the adaptive solid-state optical test target (FIG. 5B) further comprise a second layer of fluorescent dyes layered on the first layer of fluorescent dyes. In some embodiments, the second layer of fluorescent dyes comprises one type of a fluorescent dye, or a mixture of two or more different types of fluorescent dyes. In some embodiments, the fluorescent spectrum of the dye in the first coating does not substantially overlap with the fluorescence spectrum of the dye in the second coating. When the fluorescent dye coating (e.g., first layer and/or second layer) comprises a mixture of two or more different types of dyes, the fluorescent spectrum of the dyes in the mixture do not substantially overlap each other.

In some embodiments, the first layer of fluorescent dyes comprises a fluorescent dye having an excitation spectrum of 520-540 nm which can produce a green band emission of 530-630 nm. In some embodiments, the first layer of fluorescent dyes comprises a mixture of two fluorescent dyes, the first fluorescent dye having an excitation spectrum of 520-540 nm which can produce a green band emission of 530-630 nm, and the second fluorescent dye having an excitation spectrum of 630-650 nm which can produce a red band emission of 630-690 nm. In some embodiments, the first layer of fluorescent dyes comprises a first fluorescent dye having an excitation spectrum of 520-540 nm which can produce a green band emission of 530-630 nm, and the second layer of fluorescent dyes comprises a second fluorescent dye having an excitation spectrum of 630-650 nm which can produce a red band emission of 630-690 nm. See Table 1. The skilled artisan will appreciate that the excitation and emission spectrum ranges provided herein describe the portions of the spectrum in which maximum excitation and emission occur. Depending on the fluorophore, the associated spectrum may encompass tails that lie outside the ranges disclosed herein.

In some embodiments, the adaptive solid-state optical test target (FIG. 5B) is positioned in an optical imaging system which comprises at least one objective lens, at least one tube lens, and a camera. In some embodiments, the optical imaging system further comprises at least one light source (excitation source; e.g., laser and/or an LED excitation light) and at least one filter.

The present disclosure provides methods for evaluating the performance of an optical imaging system, comprising: (a) positioning the solid-state optical test target shown in FIG. 5B into an optical imaging system which comprises at least one objective lens, at least one tube lens, a camera, at least one light source (excitation source; e.g., laser) and at least one filter; (b) detecting light transmitted through a first region of a substrate of the solid-state optical test target; and (c) evaluating the performance of the optical imaging system based on the light that is transmitted through the first region of the substrate. In some embodiments, the method comprises: (a) positioning the solid-state optical test target shown in FIG. 5B into an optical imaging system which comprises at least one objective lens, at least one tube lens, a camera, at least one light source (excitation source; e.g., laser and/or an LED excitation light) and at least one filter; (b) detecting light transmitted through a first region of the first substrate; (c) detecting light transmitted through a second region of the first substrate and (d) evaluating the performance of the optical imaging system based on the light that is transmitted through the first and second regions of the first substrate.

In some embodiments, in the operation(s) of evaluating the performance of the optical imaging system, comprises any one or any combination of two or more of: determining the accuracy of the optical alignment; determining the autofocus accuracy; calibrating the light source; calibrating the camera; determining an image uniformity correction; determining distortion levels; determining contrast across a field of view; determining alignment of the camera; determining the focal distance of the camera; determining flat field correction; determining focus repeatability; determining point spread function measurement; and/or determining a modulated transfer function (MTF).

In some embodiments, the operation(s) of evaluating the performance of the optical imaging system comprises any one or any combination of two or more of: determining optical field flatness of the light source of the optical imaging system at the FOV; determining motion caused by parts of the optical system (e.g., motion caused by vibration) during imaging using the optical system; determining defocus of the optical system; determining spherical aberration and/or chromatic aberration of the optical imaging system; determining field curvature and/or image distortion of the optical imaging system; and determining the SNR and/or contrast-to-noise ratio (CNR) of the optical imaging system.

Solid-State Optical Test Targets with a Fluorescent Microscope Slide

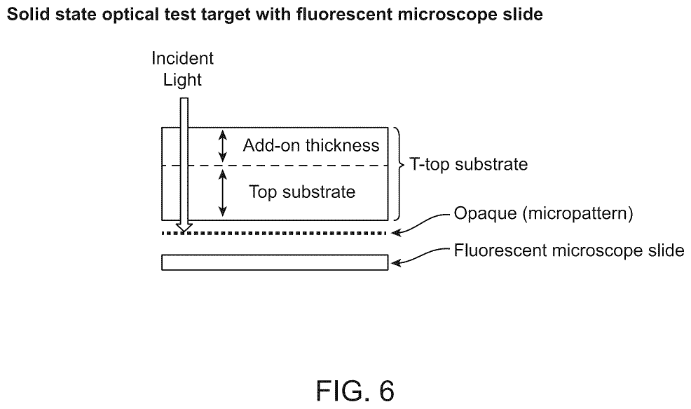

The present disclosure provides a solid-state optical test target comprising a first substrate and a second substrate, wherein the second substrate comprises a fluorescent microscope slide (FIG. 6). In some embodiments, the bottom surface of the first substrate comprises an opaque layer to form a micropattern, wherein the micropattern is configured to include opaque portions and transparent portions, as described supra. The first substrate can be positioned on the fluorescent microscope slide.

The present disclosure provides a solid-state optical test target (FIG. 6), comprising: (a) a first substrate (e.g., top glass substrate) comprising a transparent medium having even thickness, and having top, bottom and side surfaces, the top and bottom surfaces being flat, and the first substrate having a refractive index of [n-top substrate(1)]; and (b) a second substrate comprising at least a first fluorescent microscope slide. In some embodiments, the first substrate comprises transparent glass or transparent plastic. In some embodiments, at least a portion of the bottom surface of the first substrate comprises an opaque coating that forms a micropattern, the micropattern configured to include opaque portions and transparent portions. In some embodiments, the first substrate is positioned on top of the first fluorescent microscope slide. The first fluorescent microscope slide is positioned in direct contact with the micropattern on the bottom surface of the first substrate. In some embodiments, the solid-state optical test target lacks a flow cell and lacks a liquid. In some embodiments, the thickness of the first substrate is configured to simulate the presence of a first hypothetical flow cell located between the first and second substrates, wherein the first hypothetical flow cell includes a first channel having a top surface and bottom surface, and the first channel containing a designated first fluid, wherein the first channel has a first designated thickness of [T-channel(1)] and the first designated fluid has a refractive index of [n-fluid(1)]. In some embodiments, the thickness of the first substrate is configured to permit imaging of the bottom surface of the first channel of the first hypothetical flow cell.

In some embodiments, in the solid-state optical test target shown in FIG. 6, the height/thickness of the first substrate [T-top substrate(1)] is related to the refractive index of the first substrate [n-top substrate(1)], the first designated height of the first channel [T-channel(1)] and the refractive index of the first designated fluid [n-fluid(1)], in an equation:

[T-top substrate(1)]=([(T-channel(1)]*([(n-fluid(1)]/[n-top substrate(1)])) (Equation 2).

In some embodiments, the thickness of the opaque coating that forms the micropattern on the bottom surface of the first substrate (FIG. 6) is about 100 nm. In some embodiments, the thickness of the opaque coating is between about 50 nm to about 150 nm. In some embodiments, the thickness of the opaque coating that forms the micropattern on the top surface of the second substrate is between about 20 nm to about 250 nm. In some embodiments, the thickness of the opaque coating that forms the micropattern on the top surface of the second substrate is between about 40 nm to about 350 nm. In some embodiments, the thickness of the opaque coating is about 50 nm, about 60 nm, about 70 nm, about 80 nm, about 90 nm, about 100 nm, about 110 nm, about 120 nm, about 130 nm, about 140 nm or about 140 nm. In some embodiments, the micropattern on the top surface of the second substrate can be applied by vapor deposition. In some embodiments, the opaque coating comprises chromium or aluminum.

The transparent portions of the micropattern can comprise repeating shapes arranged in an array. The transparent portions of the micropattern can form a plurality of one type of shape, or a mixture of different types of shapes, wherein the one type of shape and the mixture of different types of shapes are selected from a group consisting of circles, squares and triangles. The transparent portions of the micropattern can form the shape of at least one line, or a plurality of lines arranged in parallel, or a plurality of lines arranged crossing each other, or a plurality of lines arranged in a grid pattern. The transparent portions of the micropattern can form at least one alphanumeric character. The transparent portions of the micropattern can form a plurality of pinholes. The transparent portions of the micropattern can form a plurality of pinholes and a plus sign. The pinholes can fill a full field of view when the solid-state optical test target is positioned in an optical imaging system which comprises at least one objective lens, at least one tube lens, and a camera. The transparent portions of the micropattern can form a non-repeating rotationally symmetrical shape, for example including concentric circles (e.g., bulls-eye) or a plus sign (+). In some embodiments, the dimension of any of the transparent portions of the micropattern is about 0.1 micron, or about 0.2 microns, or about 0.3 microns, or about 0.4 microns, or about 0.5 microns, or about 0.6 microns, or about 0.7 microns, or about 0.8 microns, or about 0.9 microns, or about 1 micron.

The opaque portions of the micropattern can comprise repeating shapes arranged in an array. The opaque portions of the micropattern can form a plurality of one type of shape, or a mixture of different types of shapes, wherein the one type of shape and the mixture of different types of shapes are selected from a group consisting of circles, squares and triangles. The opaque portions of the micropattern can form the shape of at least one line, or a plurality of lines arranged in parallel, or a plurality of lines arranged crossing each other, or a plurality of lines arranged in a grid pattern. The opaque portions of the micropattern can form at least one alphanumeric character. The opaque portions of the micropattern can form a plurality of pinholes. The opaque portions of the micropattern can form a plurality of pinholes and a plus sign. The pinholes can fill a full field of view when the solid-state optical test target is positioned in an optical imaging system which comprises at least one objective lens, at least one tube lens, and a camera. The opaque portions of the micropattern can form a non-repeating rotationally symmetrical shape, for example including concentric circles (e.g., bullseye) or a plus sign (+). In some embodiments, the dimension of any of the opaque portions (e.g., a length, a radius, a width, etc.) of the micropattern is about 0.1 micron, or about 0.2 microns, or about 0.3 microns, or about 0.4 microns, or about 0.5 microns, or about 0.6 microns, or about 0.7 microns, or about 0.8 microns, or about 0.9 microns, or about 1 micron.

In some embodiments, the fluorescent microscope slide provides a continuous fluorescent field and has a fluorescence spectrum. Fluorescent microscope slides include acrylic slides that are commercially-available from ThorLabs (e.g., blue (catalog No. FSK1), green (catalog No. FSK2), yellow (catalog No. FSK3), orange (catalog No. FSK4) or red (catalog No. FSK6) fluorescent microscope slides). In some embodiments, the fluorescent microscope slide has a fluorescence spectrum that produces a green band emission. In some embodiments, the second fluorescent microscope slide has a fluorescence spectrum that produces a red band emission.

In some embodiments, the solid-state optical test target shown in FIG. 6 can be positioned in an optical imaging system which comprises at least one objective lens, at least one tube lens, and a camera. The optical imaging system can further comprise at least one light source (excitation source; e.g., laser) and at least one filter.

The present disclosure provides methods for evaluating the performance of an optical imaging system, comprising: (a) positioning the solid-state optical test target shown in FIG. 6 into an optical imaging system which comprises at least one objective lens, at least one tube lens, a camera, at least one light source (excitation source; e.g., laser) and at least one filter; (b) detecting light transmitted through a substrate of the solid-state optical test target; and (c) evaluating the performance of the optical imaging system based on the light that is transmitted through the substrate.

In some embodiments the operation(s) of evaluating the performance of the optical imaging system comprises any one or any combination of two or more of: determining the accuracy of the optical alignment; determining the autofocus accuracy; calibrating the light source; calibrating the camera; determining an image uniformity correction; determining distortion levels; determining contrast across a field of view; determining alignment of the camera; determining the focal distance of the camera; determining flat field correction; determining focus repeatability; determining point spread function measurement; and/or determining a modulated transfer function (MTF).

In some embodiments, the operation(s) of evaluating the performance of the optical imaging system comprises any one or any combination of two or more of: determining optical field flatness of the light source of the optical imaging system at the FOV; determining motion caused by parts of the optical system (e.g., motion caused by vibration) during imaging using the optical system; determining defocus of the optical system; determining spherical aberration and/or chromatic aberration of the optical imaging system; determining field curvature and/or image distortion of the optical imaging system; and determining the SNR and/or CNR of the optical imaging system.

Solid-State Optical Test Targets with an LED Light

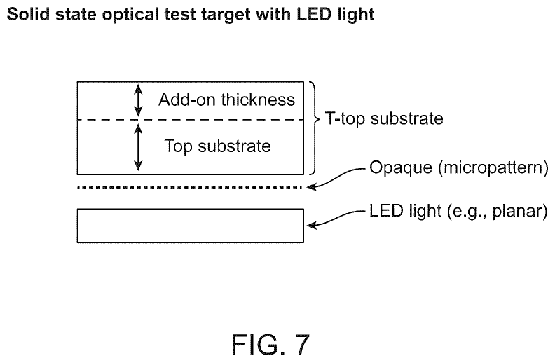

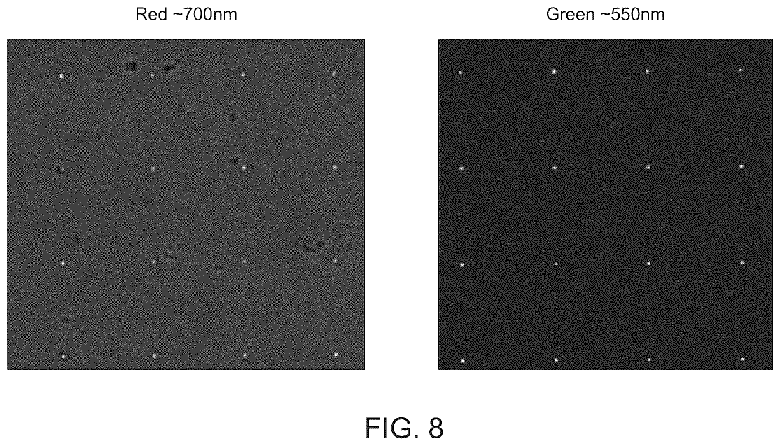

The present disclosure provides a solid-state optical test target comprising a first substrate and a second substrate, wherein the second substrate is an LED light or a plurality of LED lights (FIG. 7). In some embodiments, the bottom surface of the first substrate comprises an opaque layer to form a micropattern, wherein the micropattern is configured to include opaque portions and transparent portions. The first substrate can be positioned on the LED light (the second substrate). In some embodiments, the LED light comprises a planar-shaped LED light. The LED light can be a light source and illuminate through the bottom surface of the first substrate.

The present disclosure provides a solid-state optical test target (FIG. 7), comprising: (a) a first substrate (e.g., top substrate) comprising a transparent medium having even thickness, and having top, bottom and side surfaces, the top and bottom surfaces being flat, and the first substrate having a refractive index of [n-top substrate(1)]; and (b) a second substrate comprising an LED light, for example a planar-shaped LED light. In some embodiments, the first substrate comprises transparent glass or plastic. In some embodiments, at least a portion of the bottom surface of the first substrate comprises an opaque coating that forms a micropattern, the micropattern configured to include opaque portions and transparent portions. In some embodiments, the first substrate is positioned on top of the LED light. The LED light is positioned in direct contact with the micropattern on the bottom surface of the first substrate. In some embodiments, the solid-state optical test target lacks a flow cell and lacks a liquid. In some embodiments, the thickness of the first substrate is configured to simulate the presence of a first hypothetical flow cell located between the first and second substrates, wherein the first hypothetical flow cell includes a first channel having a top surface and bottom surface, and the first channel containing a designated first fluid, wherein the first channel has a first designated thickness of [T-channel(1)] and the first designated fluid has a refractive index of [n-fluid(1)]. In some embodiments, the thickness of the first substrate is configured to permit imaging of the bottom surface of the first channel of the first hypothetical flow cell.

In some embodiments, the LED light (e.g., 295 in FIG. 29C) may generate excitation light at different wavelengths. In some embodiments, the LED light may generate light to mimic the excitation light (e.g., color(s), light duration, energy level, etc.) in real next generation sequencing applications.

In some embodiments, the power or energy level of the LED light is customized so that the optical signal intensity emitted from the transparent portion of the optical test target is comparable to the optical signal intensity emitted from the clusters or polonies immobilized on the flow cell device during next generation sequencing applications. In some embodiments, the difference between the signal intensities being compared is less than ±1%, ±2%, ±4%, ±6%, ±8%, ±10%, ±15%, ±20%, ±25%, ±30%, or ±35%.

In some embodiments, the LED light may generate white light so that the exact excitation light conditions with different colors (e.g., blue, red, and/or green) used in next generation sequencing are transmitted to the optical test target to mimic the excitation conditions in real sequencing applications using the optical imaging system, e.g., sequencing applications using multiple image sensors for detecting light of different colors in different color channels. In some embodiments, the LED light may generate a light signal with wavelength(s) in the range from about 380 nm to 1180 nm. In some embodiments, the LED light may generate a light signal with a wavelength range that is identical to the excitation light generated by the light source of sequencing system during sequencing applications.

In some embodiments, the LED light may generate a light signal with wavelength(s) in the range from about 390 nm to 1200 nm. In some embodiments, the LED light may generate a light signal with wavelength(s) in the range from about 400 nm to 1200 nm. In some embodiments, the LED light may generate a light signal with a wavelength range that is identical to the emission light emitted by the sample(s) during sequencing applications.

In some embodiments, the LED light may generate light with different colors sequentially to mimic the excitation light condition in real sequencing application, e.g., sequencing using a single image sensor for collecting images sequentially. For example, the LED light may include a blue light source, a green light source, and a red light source that each generate excitation light in a predetermined order. Light signals at the test target after each different colored excitation light is applied may be detected for evaluating performance of the optical imaging system.

In some embodiments, LED light, instead of excitation light that may cause emission of fluorescent light (e.g., laser light) from the sample(s), may be used. The LED light described herein may advantageously separate the detectable light from the optical test target from the sample excitation path of the optical imaging system, e.g., from the light source to the sample(s). As such, the detectable light from the optical test target may not be affected by the excitation path of the optical imaging system. In other words, the optical test target may have an independent excitation path from the LED light but not from the excitation light source for exciting real samples. In some embodiments, the optical test target may share at least part of the emission path for imaging real samples.

In some embodiments, the LED light herein may advantageously remove the need to use fluorescent labels on the optical test target. In some embodiments, the LED light herein may advantageously remove the need to administer new fluorescent dyes as well as eliminate the problem of photon bleaching associated with using fluorescent dyes as in real sequencing applications.

In some embodiments, the optical test target herein may include a color balancing filter and/or diffusor that may advantageously diffuse the light in different colors to provide homogenous illumination across the FOV of the optical test target. In some embodiments, the color balancing filter and/or diffusor, e.g., 296 in FIG. 29C, may enable balancing of the power of excitation light in different colors at the FOV. In some embodiments, the variance in power (e.g., from light in two different colors from the LED light) at the FOV of the optical test target is less than ±1%, ±2%, ±5%, ±10%, ±15%, ±20%, ±25%, or ±30%.

In some embodiments, the variance in power (e.g., from light in the same color from the LED light) across the FOV of the optical test target is less than 1%, 2%, 5%, 10%, 15%, 20%, 25%, or 30%. In some embodiments, the variance in power generated by the LED light across the FOV of the optical test target is in a range from ±1% to ±35%.

In some embodiments, the FOV of the optical test target (e.g., with homogenous illumination by the LED light) is greater than 2 mm², 4 mm², 6 mm², 8 mm², 10 mm², 15 mm², 20 mm², 30 mm², 50 mm², 60 mm², 80 mm², or 100 mm².

In some embodiments, the optical lens (e.g., 297 in FIG. 29C) may be used to adjust the illumination of the LED light. In some embodiments, the optical lens may include a collimator. In some embodiments, the optical lens may include a Fresnel lens.

FIGS. 29C-29D show exemplary embodiments of positioning the optical test target relative to the LED light, color balancing filter/diffuser for evaluating one or more functions of the optical image systems. In some embodiments, the optical test target may be installed so that the excitation light path of the optical test target is different from the excitation light path, e.g., from the light source to the sample(s), in real sequencing applications. FIG. 29C shows an exemplary excitation light path from the LED light to the optical test target. With a different excitation light path, the optical test target may be positioned independently, e.g., permanently, in the optical imaging system without interfering with the real sequencing applications using the optical imaging system. Thus, the optical test target here advantageously remove the need for removing it from the optical imaging system in order to proceed properly with real sequencing applications. For example, the optical test target may be positioned at the same z-location as the flow cell(s) to be sequenced using the sequencing system but at different x and/or y location relative to the flow cell(s) to be sequenced.

In some embodiments, the optical test target shares the same emission path from the test target to the image sensor as the real sequencing applications using the optical imaging system. FIGS. 29C-29D show different exemplary embodiments of positioning the optical test target relative to the LED light and other optical parts that form the excitation path to the optical test target.

In some embodiments, in the solid-state optical test target shown in FIG. 7, the height/thickness of the first substrate [T-top substrate(1)] is related to the refractive index of the first substrate [n-top substrate(1)], the first designated height of the first channel [T-channel(1)] and the refractive index of the first designated fluid [n-fluid(1)], in an equation:

[T-top substrate(1)]=([(T-channel(1)]*([(n-fluid(1)]/[n-top substrate(1)])) (Equation 2).

In some embodiments, the thickness of the opaque coating that forms the micropattern on the bottom surface of the first substrate (FIG. 7) is about 100 nm. In some embodiments, the thickness of the opaque coating is between about 50 nm to about 150 nm. In some embodiments, the thickness of the opaque coating that forms the micropattern on the top surface of the second substrate is between about 20 nm to about 250 nm. In some embodiments, the thickness of the opaque coating that forms the micropattern on the top surface of the second substrate is between about 40 nm to about 350 nm. In some embodiments, the thickness of the opaque coating is about 50 nm, about 60 nm, about 70 nm, about 80 nm, about 90 nm, about 100 nm, about 110 nm, about 120 nm, about 130 nm, about 140 nm or about 140 nm. In some embodiments, the micropattern on the top surface of the second substrate can be applied by vapor deposition. In some embodiments, the opaque coating comprises chromium or aluminum.

The transparent portions of the micropattern can comprise repeating shapes arranged in an array. The transparent portions of the micropattern can form a plurality of one type of shape, or a mixture of different types of shapes, wherein the one type of shape and the mixture of different types of shapes are selected from a group consisting of circles, squares and triangles. The transparent portions of the micropattern can form the shape of at least one line, or a plurality of lines arranged in parallel, or a plurality of lines arranged crossing each other, or a plurality of lines arranged in a grid pattern. The transparent portions of the micropattern can form at least one alphanumeric character. The transparent portions of the micropattern can form a plurality of pinholes. The transparent portions of the micropattern can form a plurality of pinholes and a plus sign. The pinholes can fill a full field of view when the solid-state optical test target is positioned in an optical imaging system which comprises at least one objective lens, at least one tube lens, and a camera. The transparent portions of the micropattern can form a non-repeating rotationally symmetrical shape, for example including concentric circles (e.g., bulls-eye) or a plus sign (+). In some embodiments, the dimension of any of the transparent portions of the micropattern is about 0.1 micron, or about 0.2 microns, or about 0.3 microns, or about 0.4 microns, or about 0.5 microns, or about 0.6 microns, or about 0.7 microns, or about 0.8 microns, or about 0.9 microns, or about 1 micron.

The opaque portions of the micropattern can comprise repeating shapes arranged in an array. The opaque portions of the micropattern can form a plurality of one type of shape, or a mixture of different types of shapes, wherein the one type of shape and the mixture of different types of shapes are selected from a group consisting of circles, squares and triangles. The opaque portions of the micropattern can form the shape of at least one line, or a plurality of lines arranged in parallel, or a plurality of lines arranged crossing each other, or a plurality of lines arranged in a grid pattern. The opaque portions of the micropattern can form at least one alphanumeric character. The opaque portions of the micropattern can form a plurality of pinholes. The opaque portions of the micropattern can form a plurality of pinholes and a plus sign. The pinholes can fill a full field of view when the solid-state optical test target is positioned in an optical imaging system which comprises at least one objective lens, at least one tube lens, and a camera. The opaque portions of the micropattern can form a non-repeating rotationally symmetrical shape, for example including concentric circles (e.g., bulls-eye) or a plus sign (+). In some embodiments, the dimension of any of the opaque portions (e.g., a length, a radius, a width, etc.) of the micropattern is about 0.1 micron, or about 0.2 microns, or about 0.3 microns, or about 0.4 microns, or about 0.5 microns, or about 0.6 microns, or about 0.7 microns, or about 0.8 microns, or about 0.9 microns, or about 1 micron.

In some embodiments, the solid-state optical test target shown in FIG. 7 can be positioned in an optical imaging system which comprises at least one objective lens, at least one tube lens, and a camera. The optical imaging system can further comprise at least at least one filter.

The present disclosure provides methods for evaluating the performance of an optical imaging system, comprising: (a) positioning the solid-state optical test target shown in FIG. 7 into an optical imaging system which comprises at least one objective lens, at least one tube lens, a camera, and at least one filter; (b) detecting light transmitted through a substrate of the solid-state optical test target; and (c) evaluating the performance of the optical imaging system based on the light that is transmitted through the substrate.

In some embodiments the operation(s) of evaluating the performance of the optical imaging system, comprises any one or any combination of two or more of: determining the accuracy of the optical alignment; determining the autofocus accuracy; calibrating the light source; calibrating the camera; determining an image uniformity correction; determining distortion levels; determining contrast across a field of view; determining alignment of the camera; determining the focal distance of the camera; determining flat field correction; determining focus repeatability; determining point spread function measurement; and/or determining a modulated transfer function (MTF).

In some embodiments, the operation(s) of evaluating the performance of the optical imaging system comprises any one or any combination of two or more of: determining optical field flatness of the light source of the optical imaging system at the FOV; determining motion caused by parts of the optical system (e.g., motion caused by vibration) during imaging using the optical system; determining defocus of the optical system; determining spherical aberration and/or chromatic aberration of the optical imaging system; determining field curvature and/or image distortion of the optical imaging system; and determining the SNR and/or CNR of the optical imaging system.

### Flow Cell Devices

Disclosed herein, in some embodiments, are flow cell devices that can be employed for performing or facilitating DNA sequencing analysis. The flow cell devices can be used for evaluating performance of various sequencing systems, especially the optical system therewithin. The flow cell devices can be used for calibration of various sequencing systems and their optical systems. The flow cell devices can be used during sequencing and enable image registration of flow cell images acquired with or without the need of fiducial markers external to the flow cell devices. The flow cell devices can be used during sequencing to enable real-time or near real-time image registration of flow cell images.

The flow cell devices disclosed herein can include a plurality of fluorescent beads that emit light when exposed to laser or other forms of light excitation. The flow cell devices with the plurality of fluorescent beads can mimic the distribution, signal intensity, and/or color of clusters or polonies during sequencing runs. As such, the flow cell devices with the plurality of fluorescent beads can be imaged using a sequencing system for calibration of the sequencing system or for evaluating the performance of the sequencing system. The flow cell device can also be imaged during sequencing runs to provide fiducial markers for image registration of clusters or polonies across different channels or cycles.

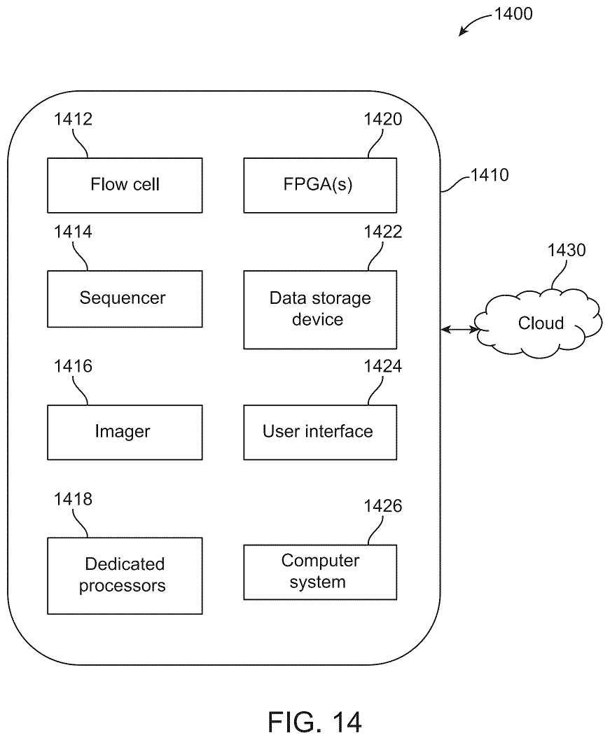

In some embodiments, calibration of a sequencing system 1410 (shown in FIG. 14) comprises evaluating the performance of the sequencing system 1410. Evaluating the performance can include but is not limited to one or more of: determining the accuracy of the optical alignment; determining the autofocus accuracy; calibrating the light source; calibrating the camera; determining an image uniformity correction; determining distortion levels; determining contrast across a field of view; determining alignment of the camera; determining the focal distance of the camera; determining flat field correction; determining focus repeatability; determining point spread function measurement; and determining a modulated transfer function (MTF).

In some embodiments, calibration of the sequencing system 1410 comprises calibrating one, two, three, four, five, or six color channels of the sequencing system 1410. In some embodiments, calibration of the sequencing system 1410 comprises evaluating performance of the optical system with respect to the one, two, three, four, five, or six color channels.

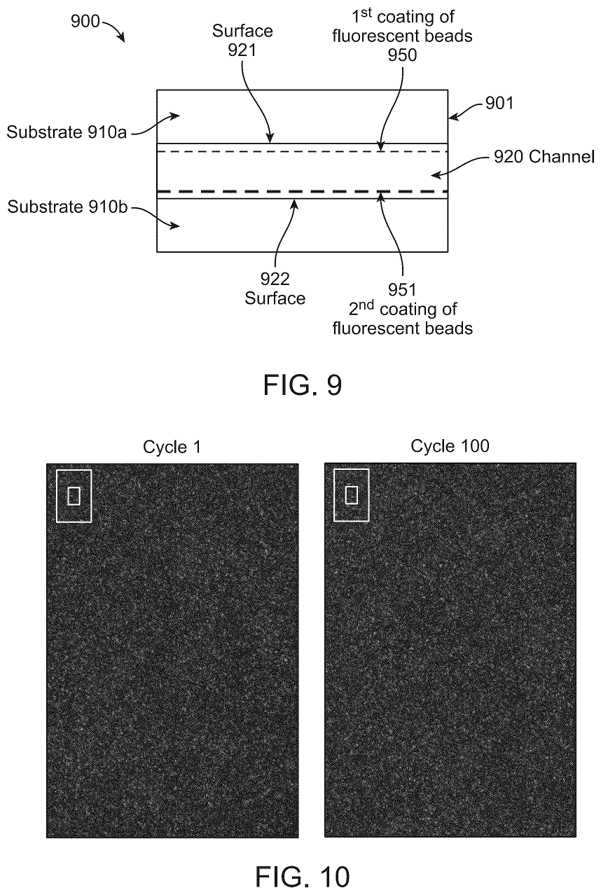

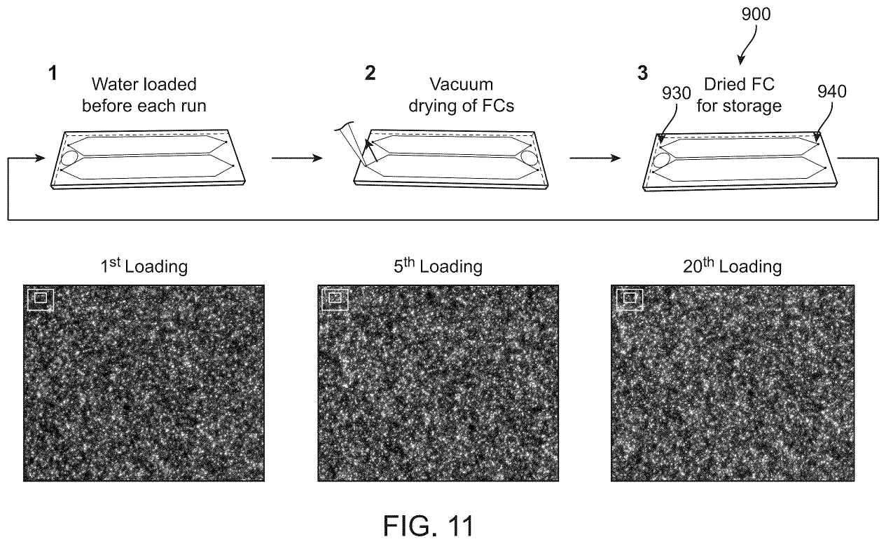

In some embodiments, a flow cell device disclosed herein can comprise a support disclosed herein. The support can be solid. At least part of the support can be transparent. The support can comprise one or more substrates. At least part of the one or more substrate can be transparent. FIG. 9 shows an exemplary embodiment of the flow cell device 900. The flow cell device 900 includes a support 901 and other flow cell compounds such as coatings. The support can comprise a top substrate 910a and a bottom substrate 910b. Each substrate 910 can have a predetermined thickness, and different substrate can have different thickness. The substrate can define one or more channels 920 of the device 900. The channels 920 can allow fluid flow therethrough, e.g., liquid or air. FIG. 11 shows the flow cell device 900 can include one or more inlets 930 and one or more outlets 940 in the one or more substrate 910. FIGS. 9 and 11 show exemplary devices 900 with two substrates forming two channels, and each channel having an inlet and an outlet. However, the number of substrates, channels, inlets and outlets in other embodiments can be different. In some embodiments, the flow cell devices 900 can have any suitable number of substrates, channels, inlets and outlets. FIGS. 9 and 11 show exemplary device 900 with two planar substrates without curvature in the surface(s) of the substrate. However, the substrate does not have to be planar.

In some embodiments, the support, and/or the one or more substrates can comprise glass or plastic. In some embodiments, one or more substrates are all-glass or all-plastic.

In some embodiments, as shown in FIGS. 9 and 11, the one or more channels 920 can run from the inlet 930 to the outlet 940 so that fluid can flow from the inlet 930 via the one or more channels 920 to the outlet 940. As an example, sequencing reagents can be introduced to the flow cell device via the inlet, flow through the channels, and then exit from the outlet. The channel(s) 920 can comprise a top interior surface 921 and a bottom interior surface 922. One or more of the surfaces can be coated with a plurality fluorescent beads.

The plurality of fluorescent beads can be chemically immobilized to the surface. The plurality of fluorescent beads can be covalently immobilized to the surface. The plurality of fluorescent beads can be immobilized or fixedly attached to the surface 921, 922 by forming a coating 950, 951 thereon, so that the fluorescent beads remain fixed or immobilized relative to the surface 921, 922. The coating 950, 951 can be applied directly to and in contact with the interior surface 921, 922. Alternatively, the coating 950, 951 can be applied indirectly to or not in direct contact with the interior surface 921, 922. In some embodiments, the coating 950, 951 can be applied with some compounds in between the surface 921, 922 and the coating 950, 951. For example, the coating 950, 951 can be applied on top of another coating that is directly applied to and in contact with the surface 921, 922. In some embodiments, the surface is passivated with another coating (not shown). The another coating can immobilize surface capture primers, nucleic acid template molecules, or both for capturing polynucleotides on the surface 921, 922. In some embodiments, the surface 921, 922 comprises polynucleotides captured thereon.

In some embodiments, the coating 951, 952 that attaches the fluorescent beads can be mixed with one or more other coatings so that the mixed coating can be applied directly to and in contact with the interior surface 921, 922. The mixed coating can immobilize fluorescent beads on the surface. Further, the mixed coating may also immobilize surface capture primers, nucleic acid template molecules, or both for capturing polynucleotides on the surface 921, 922. In some embodiments, the fixed coating may capture polynucleotides on the surface 921, 922, and administration of sequencing reagents can facilitate sequencing of the polynucleotides as disclosed herein using various sequencing methods, for example, sequencing-via-avidite.

In some embodiments, the flow cell device 900 can be used on a sequencing system 1410 for DNA sequencing. The flow cell device 900 may receiving various sequencing reagents before a sequencing cycle via the inlet 930 and allow the reagent(s) to flow through the one or more channels 920 and exit via the outlet 940. In some embodiments, the fluorescent beads remain immobilized relative to the surface 921, 922 during or after administration of sequencing reagents to the flow cell device 900.

In some embodiments, the plurality of fluorescent beads are chemically immobilized to the surface. In some embodiments, the fluorescent beads are covalently immobilized to the surface. In some embodiments, the fluorescent beads are pre-activated to enable chemical attachment to the surface. In some embodiments, the fluorescent beads are pre-activated to enable covalent attachment to the surface.

In some embodiments, the clusters or polonies of polynucleotides captured thereon and the fluorescent beads are imaged simultaneously in one or more sequencing cycles using a sequencing system 1410. In some embodiments, a flow cell image acquired of the flow cell device 900 can include signals from the fluorescent beads and signals from clusters or polonies of polynucleotides. Since the fluorescent beads are immobilized to the surface, their positions relative to the surface remain fixed, while the polynucleotides may move relative to the surface, even if they are captured on the surface. The movement of the polynucleotides in different flow cell images may cause errors in base calling, thereby requiring image registration before base calling. The immobilized fluorescent beads can be used as fiducial markers for registering or aligning clusters or polonies across different cycles and/or different channels and eliminate the need of using additional fiducial markers that is external to the flow cell device, such as the optical test targets disclosed herein. In some embodiments, the positions of same fluorescent beads in two different images across cycles and/or channels can be used to determine a transformation between the two different flow cell images, and the transformation can be applied to the positions of the clusters or polonies to perform image registration of the clusters or polonies. The transformation can include one or more transformation matrixes for some area or the entire flow cell images. In embodiments where at least some of the clusters or polonies move across cycles and/or channels, the flow cell device disclosed herein can advantageously facilitate image registration of flow cell images and allow more accurate and reliable base calling after image registration.

In some embodiments, the clusters or polonies and the fluorescent beads can be imaged in different cycles. For example, the cluster or polonies can be imaged during bright cycles while the fluorescent beads can be imaged in a dark cycle.

In some embodiments, the clusters or polonies and the fluorescent beads can be imaged in same cycle(s). For example, the clusters or polonies and the fluorescent beads can be imaged simultaneously in a first cycle and an nth cycle, wherein the number n can be any integer greater than 1. Since the fluorescent beads remain fixed to the surface, the change in positions of the fluorescent beads between cycles can be used to determine movements that can be attributed to sources in the sequencing system and external to the flow cell device, such as movement of the stage, an optical element, etc. The positions of the clusters or polonies relative to the fluorescent beads can be used to determine movements attributed to motion of the clusters or polonies on the surface, so that the clusters or polonies in the first and the nth cycle can be aligned/registered independent of movement attributable to cause(s) external to the flow cell devices.

In some embodiments, the clusters or polonies and the fluorescent beads can be imaged in same channel(s) of identical or different cycles so that image transformation caused by difference in elements of the sequencing system or other variances across cycles are determined in aligning/registering the clusters or polonies in different flow cell images. In some embodiments, the clusters or polonies and the fluorescent beads can be imaged in different channels of identical or different cycles so that image transformation can take into consideration transformation across channels, alone or in combination with transformation across cycles.

In some embodiments, the flow cell device 900 with fluorescent beads can be used for image registration in combination with the solid-state optical test target disclosed herein. For example, the solid-state optical test target can be used for registering images from different channels within a same cycle and the fluorescent beads can be used for registering images from different cycles but identical channel(s). As another example, the solid-state optical test target can be used for registering images from a reference cycle to a template coordinate system, and fluorescent beads can be used for registering images acquired from all channels in a cycle that is not a reference cycle to the temple coordinate system.

In some embodiments, the plurality of fluorescent beads can comprise one, two, three, four, five or six different types of beads that emits different colors or wavelength of light in response to a laser light or otherwise light excitement. In some embodiments, the different types of beads can be about equal in their total amounts that are attached to the surface.

In some embodiments, at least a portion of the polynucleotides move from first positions in a first flow cell image acquired via a first channel in a first sequencing cycle to second positions in a second flow cell image acquired via a second channel in the first sequencing cycle and the fluorescent beads remain immobilized relative to the surface in the first and second flow cell images. The first and second channel can be identical or different. The first and second sequencing cycle can be identical or different.

The image registration using the flow cell device herein can enable registration/alignment of the first flow cell image and the second flow cell image, for example, by aligning or registering the images to a common coordinate system so that the polynucleotides appear at updated positions after image registration and base calling can be reliably and accurately performed based on the updated positions. A first image transformation can be determined from the first positions in the first flow cell image to the updated position, and a second image transformation can be determined from the second positions to the updated position. Alternatively, an image transformation can be determined between the first positions and the second positions of two flow cell images. The positions of the fluorescent beads in the first and second flow cell images can be used to determine image transformation of the polynucleotides. For example, since the beads are immobilized to the surface, if a polony moved further away from a specific bead in the second flow cell image, that relative movement can be attributed to the movement of the polony relative to the surface.

In some embodiments, the plurality of fluorescent beads emit first fluorescent light in response to laser light or otherwise light excitement in a first sequencing cycle in a first sequencing run. The first fluorescent light comprises a first wavelength, a first intensity, a first color, or their combinations.

In some embodiments, the plurality of fluorescent beads emit second fluorescent light in response to laser or otherwise light excitement in an nth sequencing cycle in the first sequencing run, where n is greater than 1. In some embodiments, the nth sequencing cycle is a 100^{th} cycle, a 110^{th} cycle, a 120^{th} cycle, or a 130^{th} cycle. In some embodiments, n is an integer that is greater than about 50. In some embodiments, n is an integer that is greater than about 100. In some embodiments, n is an integer that is greater than about 150. In some embodiments, n is an integer that is in a range from about 100 to about 160. The second fluorescent light can comprise a second wavelength, a second intensity, a second color, or their combinations. In some embodiments, the plurality of fluorescent beads immobilized to the surface are stable so that they are not photon bleached by repeated light excitement from the first sequencing cycle to at least the nth sequencing cycle. In some embodiments, the second intensity is about less than 10%, 8%, or 5% different from the first intensity. FIG. 10 shows fluorescence images recorded with the flow cell device including fluorescent beads at cycle 1 and cycle 100 in a sequencing run. The distribution of fluorescent beads is random with no obvious aggregation of beads for more than about 5, 10, 15, 20, 25, 30, or 40 pixels, with a spatial resolution in the range of about 0.05 μm to 0.5 um.

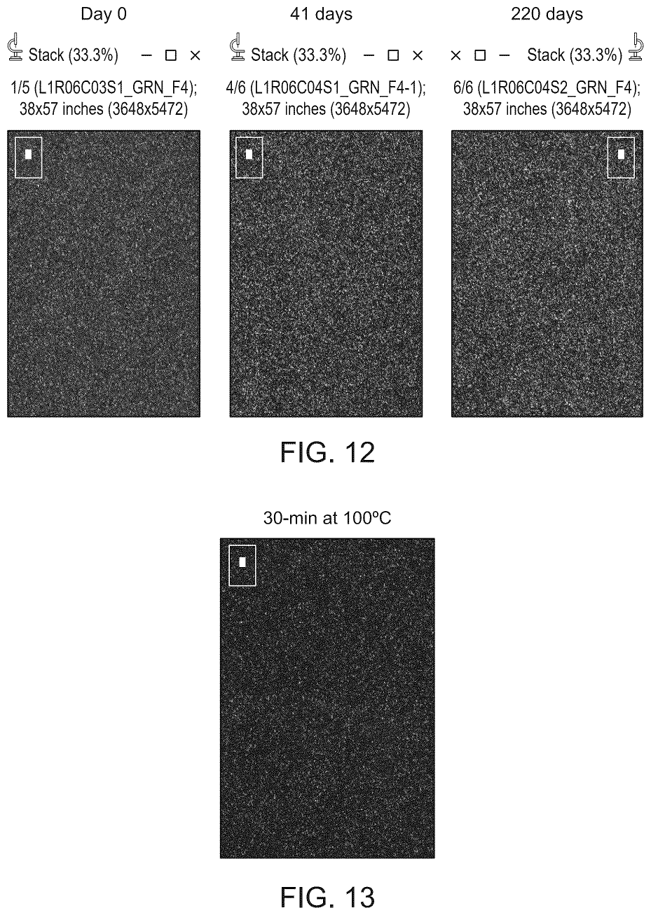

In some embodiments, the plurality of fluorescent beads emit third fluorescent light in response to laser excitement in a first or nth sequencing cycle of a second sequencing run after storing the flow cell device for about 6 months at about a room temperature after the first sequencing run. In some embodiments, the flow cell device has a shelf light at room temperature for more than about 6 months. In some embodiments, the flow cell device is stored at room temperature after drying. The third fluorescent light can comprise a third wavelength, a third intensity, a third color, or their combinations. In some embodiments, the third intensity is about less than 10%, 8%, or 5% different from the first intensity. FIG. 12 shows that the images of a flow cell device with fluorescent beads at day 0, and day 41 and day 220 in different sequencing runs. The distribution of fluorescent beads appears to remain random without apparent aggregation of beads, e.g., large spots of bright signals with more than about 5, 10, 15, 20, 25, 30, or 40 pixels, with a spatial resolution of about 0.05 μm to 0.5 um.

In some embodiments, the plurality of fluorescent beads emit fourth fluorescent light in response to laser or otherwise light excitement in a sequencing cycle in a third sequencing run after exposing the flow cell device 900 for 30 minutes in an 100° C. environment after the first sequencing run. The fourth fluorescent light comprises a fourth wavelength, a fourth intensity, a fourth color, or their combinations. In some embodiments, the fourth intensity is about less than 10%, 8%, or 5% different from the first intensity. FIG. 13 shows a flow cell image of the flow cell device with fluorescent beads after about 30 minutes in an environment of 100° C. The distribution of fluorescent beads appears to remain random without apparent aggregation of beads, e.g., large spots of bright signals with more than about 5, 10, 15, 20, 25, 30, or 40 pixels, with a spatial resolution of about 0.05 um to 0.5 um.

The plurality of fluorescent beads can emit fifth fluorescent light in response to laser excitement in a sequencing cycle in a fourth sequencing run after drying the flow cell device and refilling the flow cell with reagents. Optionally the flow cell device is refilled with flow cell reagents for more than 20 times after the first sequencing run. The fifth fluorescent light comprises a fifth wavelength, a fifth intensity, a fifth color, or their combinations. In some embodiments, the fifth intensity is about less than 10%, 8%, or 5% different from the first intensity.

In some embodiments, the first fluorescent light is from a first channel. In some embodiments, the plurality of fluorescent beads can emit sixth fluorescent light in response to laser excitement in a sequencing cycle in a second channel in the first sequencing run. In some embodiments, the sixth fluorescent light comprises a sixth wavelength, a sixth intensity, a sixth color, or their combinations. In some embodiments, the sixth intensity is about less than 10%, 8%, or 5% different from the first intensity.

In some embodiments, the first, second, third, fourth, fifth, or sixth wavelength is within a range from about 150 nm to about 850 nm. In some embodiments, the first, second, third, fourth, fifth, or sixth wavelength is within a range from about 180 nm to about 800 nm. In some embodiments, two or more of the first, second, third, fourth, fifth, and sixth wavelength are identical. In some embodiments, two or more of the first, second, third, fourth, fifth, and six wavelength are about identical. In some embodiments, the first and the sixth wavelength is different, and the first and the sixth color is different. In some embodiments, one or more of the first, second, third, fourth, fifth, or sixth color is red, green, blue, yellow, or their combinations. In some embodiments, two or more of the first, second, third, fourth, fifth, and sixth colors are about identical. In some embodiments, two or more of the first, second, third, fourth, fifth, and sixth colors are identical. In some embodiments, two or more of the first, second, third, fourth, fifth, and sixth colors are different.

In some embodiments, the plurality of fluorescent beads are about randomly distributed on the surface. In some embodiments, the plurality of fluorescent beads are distributed on the surface without aggregation. In some embodiments, the distribution of fluorescent beads appear random with no obvious aggregation of beads for an area larger than about 0.1 um\hat{ }2, about 0.5 um\hat{ }2, about 0.8 um\hat{ }2, 1 about um\hat{ }2, about 1.5 um\hat{ }2, about 2 um\hat{ }2, about 2.5 um\hat{ }2, about 3 um\hat{ }2, about 4 um\hat{ }2, about 5 um\hat{ }2, about 6 um\hat{ }2, about 7 um\hat{ }2, about 8 um\hat{ }2, about 9 um\hat{ }2 or about 10 um\hat{ }2. The distribution of fluorescent beads appear to remain random without apparent aggregation of beads, e.g., large spots of bright signals with more than about 2, 3, 4, 5, 8, 9, 10, 12, 14, 15, 20, 25, 30, 35, or 40 pixels. The spatial resolution can be in the range of about 0.01 um to about 1 um. The spatial resolution can be in the range of about 0.05 um to 0.9 um. The spatial resolution can be in the range of about 0.1 um to about 0.5 um. In some embodiments, the distribution of fluorescent beads appears random with no obvious aggregation of beads. The obvious aggregation can be determined by image analysis and determine if the number of beads within a pixel or a unit area is greater than a predetermined threshold. For example, there is no obvious aggregation of beads if less than about 100 or about 200 beads are in an area of about 3 um\hat{ }2. As another example, there is no obvious aggregation of beads if less than about 5, 8, or 10 beads are within each pixel, with a pixel resolution of about 0.3 um, about 0.4 um, or about 0.5 um.

In some embodiments, at least some or all of the imaging areas of the surface are coated so that the fluorescent beads can distributed about randomly over the coated the regions of the surface.

In some embodiments, the coating may be selectively applied in a patterned fashion to the surface so that the fluorescent beads only distribute in areas of the patterned coated areas. The fluorescent cells can randomly distribute within the areas of the patterned coating. For example, the surface may be coated in repeated circular or rectangular shapes spread with a fixed distance in between two adjacent shapes so that the fluorescent beads distribute about randomly only within the shapes.

In some embodiments, each imaging area on the surface comprises about 50,000 to about 500,000 fluorescent beads. In some embodiments, each imaging area on the surface comprises about 100,000 to about 600,000 fluorescent beads. In some embodiments, each imaging area on the surface comprises about 140,000 to about 400,000 fluorescent beads. In some embodiments, each imaging area comprises one or more tiles of the flow cell device. In some embodiments, each imaging area comprises a single tile of the flow cell device. In some embodiments, each imaging area comprises one or more subtiles of the flow cell device. In some embodiments, each imaging area comprises a single subtile or a portion of the single subtile of the flow cell device. For example, an imaging area can include a size of about 0.5, 0.2, or 0.1 of a single subtile.

In some embodiments, the fluorescent beads comprise microspheres loaded with fluorescent dyes. In some embodiments, the fluorescent beads comprise microspheres with a diameter of about 0.1 μm to about 1.5 um. In some embodiments, the fluorescent beads comprise microspheres with a diameter of about 0.2 μm to about 1.0 μm. In some embodiments, the fluorescent beads comprise microspheres with a diameter of about 0.3 μm to about 0.5 um.

In some embodiments, the fluorescent beads comprise quantum dots. In some embodiments, the quantum dots are not photobleached after repeated light excitement after n cycles, where n is a number that is greater than about 100, 150, 200, or more.

In some embodiments, the fluorescent beads comprise microspheres or particles of various shapes, e.g., nano particles, that may emit light in response to laser or otherwise light excitement.

In some embodiments, the number of fluorescent beads within an area of the surface is controlled so that the image intensity of randomly distributed fluorescent beads can be no more than about 200%, 100%, 80%, 60%, 50%, 40%, 30%, 20%, or 10% different from the image intensity of the polynucleotides. The image intensity of fluorescent beads can be average image intensity of some or all of the beads within a predetermined area. The image intensity of the polynucleotides can be average image intensity of some or all of the polynucleotides within a predetermined area. Alternatively, the image intensity can be the maximum intensity, or median intensity, or an intensity at a predetermined percentile, e.g., of 90^{th} percentile. In some embodiments, the image intensity can be significantly higher or lower than the intensity of the polynucleotides, e.g., 3 times, 5 times, 10 times or even more of the intensity of the polynucleotides.

In some embodiments, the first, second, third, fourth, fifth, and/or sixth wavelength, the first, second, third, fourth, fifth, and/or sixth color, the first, second, third, fourth, fifth, and/or sixth intensity, or their combinations can be used to enable image registration of clusters or polonies imaged using a sequencing system 1410 across different sequencing cycles or using different color channels.

In some embodiments, the first, second, third, fourth, fifth, and/or sixth wavelength, the first, second, third, fourth, fifth, and/or sixth color, the first, second, third, fourth, fifth, and/or sixth intensity, or their combinations can be used to enable calibration of the sequencing system 1410 or elements included therein, e.g., the imager 1416.

Disclosed herein are methods of using the flow cell devices 900 for performing and/or facilitating sequencing analysis. The method can include some or all of the operations disclosed herein. The operations may be performed in but is not limited to the order that is described herein.

The methods can be performed by one or more processors disclosed herein. In some embodiments, the processor can include one or more of: a processing unit, an integrated circuit, or their combinations. For example, the processing unit can include a central processing unit (CPU) and/or a graphic processing unit (GPU). The integrated circuit can include a chip such as a field-programmable gate array (FPGA). In some embodiments, the processor can include the computing system.

In some embodiments, some or all operations in the methods can be performed by the FPGA(s). In embodiments when some operations are performed by FPGA(s), the data after an operation performed by the FPGA(s) can be communicated by the FPGA(s) s to the CPU(s) so that CPU(s) can perform subsequent operation(s) in method using such data. Similarly, data can also be communicated from the CPU(s) to the FPGA(s) for processing by the FPGA(s). In some embodiments, all the operations in the method can be performed by CPU(s). Alternatively, the operations performed by CPU(s) can be performed by other processors such as the dedicated processors, or GPU(s). In some embodiments, all the operations in method can be performed by FPGA(s).

The methods disclosed herein can be used for evaluating performance of the sequencing system 1410, for calibration of the sequencing system 1410, and/or for image registration of images of clusters or polonies acquired using the sequencing system 1410. The methods can include an operation of generating first flow cell images by imaging the flow cell device using the sequencing system in a first sequencing cycle via a first channel. The flow cell device can have a plurality of fluorescent beads immobilized thereon and cluster or polonies (polynucleotides) attached thereon.

The methods can include an operation of generating second flow cell images by imaging the flow cell device using the sequencing system in a second sequencing cycle using a second channel.

The methods can include an operation of calibrating the sequencing system by analyzing the first flow cell images and the second flow cell images.

The methods can include an operation of performing image registration by analyzing the first flow cell images and the second flow cell images.

In some embodiments, analyzing the first flow cell images and the second flow cell images comprises an operation of determining the positions of fluorescent beads in the plurality of fluorescent beads in the first flow cell images and/or the second flow cell images. The positions can include image coordinates of fluorescent beads in the plurality. In some embodiments, analyzing the first flow cell images and the second flow cell images comprises an operation of determining image intensity of fluorescent beads in the plurality. In some embodiments, analyzing the first flow cell images and the second flow cell images comprises an operation of determining a center of each bright spot in flow cell images corresponding to some of the fluorescent beads in the plurality.

In some embodiments, analyzing the first flow cell images and the second flow cell images comprises an operation of determining the positions, image intensity, and center of the clusters or polonies in the first flow cell images and/or the second flow cell images.

In some embodiments, the fluorescent beads do not overlap with polynucleotides on the surface. In some embodiments, the fluorescent beads do not partially overlap with polynucleotides on the surface. In some embodiments, the fluorescent beads may partially overlap with polynucleotides on the surface. When a bead or a couple of beads partially overlap with polynucleotides, two or more bright spots in the flow cell images may partially overlap with each other. Computer-implemented methods or algorithms can be used to identify the center(s) of the overlapped bright spots in flow cell images. For example, methods disclosed in U.S. Pat. No. 11,200,446 can be used for identifying centers of bright spots in the flow cell images so that the signal from beads and nucleotides can be separated, and the spatial location of the beads and polynucleotides can be determined.

In some embodiments, analyzing the first flow cell images and the second flow cell images comprises an operation of determining one or more transformations between the first flow cell images and the second flow cell images. The operation of determining one or more transformations between the first flow cell images and the second flow cell images can be based on the positions, image intensity, and/or center of the fluorescent beads, the clusters or polonies, or both.

In some embodiments, analyzing the first flow cell images and the second flow cell images comprises an operation of determining one or more transformations between the first flow cell images and a template image and/or the second flow cell images to the template image.

FIG. 14 illustrates a block diagram of a computer-implemented system 1400, according to one or more embodiments disclosed herein. The system 1400 has a sequencing system 1410 that includes a flow cell 1412, a sequencer 1414, an imager 1416, data storage 1422, and user interface 1424. The sequencing system 1410 may be connected to a cloud 1430. The sequencing system 1410 may include one or more of dedicated processors 1418, Field-Programmable Gate Array(s) (FPGAs) 1420, and a computer system 1426.

In some embodiments, the flow cell 1412 is configured to capture DNA fragments and form DNA sequences for base-calling on the flow cell. The flow cell 1412 can include a support as disclosed herein. The support can be a solid support. The support can include a surface coating thereon as disclosed herein. The surface coating can be a polymer coating as disclosed herein.

A flow cell 1412 can include multiple tiles or imaging areas thereon, and each tile may be separated into a grid of subtiles. Each subtile can include a plurality of clusters or polonies thereon. As a nonlimiting example, a flow cell can have 424 tiles, and each tile can be divided into a 6×9 grid, therefore 54 subtiles. The flow cell image as disclosed herein can be an image including signals of a plurality of clusters or polonies. The flow cell image can include one or more tiles of signals or one or more subtiles of signals. In some embodiments, a flow cell image can be an image that includes all the tiles and approximately all signals thereon. The flow cell image can be acquired from a channel during an imaging or sequencing cycle using the imager 1416. In some embodiments, each tile may include millions of polonies or clusters. As a nonlimiting example, a tile can include about 1 to 10 million of clusters or polonies. Each polony can be a collection of many copies of DNA fragments.

The sequencer 1414 may be configured to flow a nucleotide mixture onto the flow cell 1412, cleave blockers from the nucleotides in between flowing steps, and perform other steps for the formation of the DNA sequences on the flow cell 1412. The nucleotides may have fluorescent elements attached that emit light or energy in a wavelength that indicates the type of nucleotide. Each type of fluorescent element may correspond to a particular nucleotide base (e.g., A, G, C, T). The fluorescent elements may emit light in visible wavelengths. In some embodiments, the sequencer 1414 and the flow cell 1412 may be configured to performing various sequencing methods disclosed herein, for example, sequencing-by-avidite.

For example, each nucleotide base may be assigned a color. Different types of nucleotides can have different colors. Adenine (A) may be red, cytosine (C) may be blue, guanine (G) may be green, and thymine (T) may be yellow, for example. The color or wavelength of the fluorescent element for each nucleotide may be selected so that the nucleotides are distinguishable from one another based on the wavelengths of light emitted by the fluorescent elements.

The imager 1416 may be configured to capture images of the flow cell 1412 after each flowing step. In an embodiment, the imager 1416 is a camera configured to capture digital images, such as a CMOS or a CCD camera. The camera may be configured to capture images at the wavelengths of the fluorescent elements bound to the nucleotides. The images can be called flow cell images.

In some embodiments, the imager 1416 can include one or more optical systems disclose herein. The optical system(s) can be configured to capture optical signals from the flow cell and generate corresponding digital images thereof. The digital images can then be used for base calling.

In an embodiment, the images of the flow cell may be captured in groups, where each image in the group is taken at a wavelength or in a spectrum that matches or includes only one of the fluorescent elements. In another embodiment, the images may be captured as single images that captures all of the wavelengths of the fluorescent elements.

The resolution of the imager 1416 controls the level of detail in the flow cell images, including pixel size. In existing systems, this resolution is very important, as it controls the accuracy with which a spot-finding algorithm identifies the polony centers. In some embodiments, the image resolution of flow cell images disclosed herein can be about 10 nanometers (nms or nm) to a couple of hundreds of nm or greater. One way to increase the accuracy of spot finding is to improve the resolution of the imager 1416, or improve the processing performed on images taken by imager 1416. Detecting polony centers in pixels other than those detected by a spot-finding algorithm can be performed. These methods can allow for improved accuracy in detection of polony centers without increasing the resolution of the imager 1416. The resolution of the imager may even be less than existing systems with comparable performance, which may reduce the cost of the sequencing system 1410.

The image quality of the flow cell images controls the base calling quality. One way to increase the accuracy of base calling is to improve the imager 1416, or improve the processing performed on images taken by imager 1416 to result in a better image quality. The methods described herein register the flow cell images to a common coordinate system so that the base calling with respect to a cluster or polony can be more accurate than without such registration. These methods can allow for image registration during sequencing of clusters and polonies. Further, since the methods disclosed here are computationally less intensive than traditional methods so that the heat dissipation by the computer/processors can be easier to manage so that it is unlikely to cause undesired shift from the proper chemistry of sequencing techniques disclosed herein. These methods can be advantageously performed in parallel in the computer-implemented system 1400, without interference with or delay of existing sequencing workflow of the system 1400. The results of image registration can be available for making actual base calling in the current cycle in the sequencing workflow. Further, some or all of the operations disclosed herein can be advantageously performed by the FPGA(s) and data can be communicated between the CPU(s) and FPGA(s) to reduce the total operational time from methods operating without the FPGA(s). Yet further, instead of directly registering multiple flow cell images which may require saving the images before and/or after registration, image intensities and corresponding positions of selected polonies are extracted to estimate the transformation of the entire flow cell image. Further, transformation matrixes instead of images can be saved, which can save memory space needed and improve efficiency of the image registration process.

The sequencing system 1410 may be configured to perform image registration the flow cell images across different cycles and/or channels. The operations or actions disclosed herein may be performed by the dedicated processors 1418, the FPGA(s) 1420, the computing system 1426, or a combination thereof. One or more operations or actions in the methods disclosed herein may be performed by the dedicated processors 1418, the FPGA(s) 1420, the computing system 1426, or a combination thereof. In some embodiments, which operations or actions are to be performed by performed by the dedicated processors 1418, the FPGA(s) 1420, the computer system 1426, or their combinations can be determined based on one or more of: a computation time for the specific operation(s), the complexity of computation in the specific operation(s), the need for data transmission between the hardware devices, or their combinations. Image registration disclosed herein can be performed after the flow cell images are acquired but before actual base calling of the flow cell images is performed in a cycle.

The computer system 1426 can include one or more general purpose computers that provide interfaces to run a variety of program in an operating system, such as Windows™ or Linux™. Such an operating system typically provides great flexibility to a user.

In some embodiments, the dedicated processors 1418 may be configured to perform operations in the methods herein. They may not be general-purpose processors, but instead custom processors with specific hardware or instructions for performing those steps. Dedicated processors directly run specific software without an operating system. The lack of an operating system reduces overhead, at the cost of the flexibility in what the processor may perform. A dedicated processor may make use of a custom programming language, which may be designed to operate more efficiently than the software run on general-purpose computers. This may increase the speed at which the steps are performed and allow for real time processing.

In some embodiments, the FPGA(s) 1420 may be configured to perform operations included in the methods herein. An FPGA is programmed as hardware that will only perform a specific task. A special programming language may be used to transform software steps into hardware componentry. Once an FPGA is programmed, the hardware directly processes digital data that is provided to it without running software. The FPGA instead uses logic gates and registers to process the digital data. Because there is no overhead required for an operating system, an FPGA generally processes data faster than a general-purpose computer. Similar to dedicated processors, this is at the cost of flexibility.

The lack of software overhead may also allow an FPGA to operate faster than a dedicated processor, although this will depend on the exact processing to be performed and the specific FPGA and dedicated processor.

A group of FPGA(s) 1420 may be configured to perform the steps in parallel. For example, a number of FPGA(s) 1420 may be configured to perform a processing step for an image, a set of images, a subtile, or a select region in one or more images. Each FPGA(s) 1420 may perform its own part of the processing step at the same time, reducing the time needed to process data. This may allow the processing steps to be completed in real time. Further discussion of the use of FPGAs is provided below.

Performing the processing steps in real time may allow the system to use less memory, as the data may be processed as it is received. This improves over conventional systems may need to store the data before it may be processed, which may require more memory or accessing a computer system located in the cloud 1430.

In some embodiments, the data storage device 1422 is used to store information used in the methods. This information may include the images themselves or information derived from the images captured by the imager 1416. The DNA sequences determined from the base-calling may be stored in the data storage device 1422. Parameters identifying polony locations may also be stored in the data storage device 1422. Raw and/or processed image intensities of each polony may be stored in the data storage device 1422. The region and/or subtile that each polony corresponds to may also be stored in the data storage device 1422. The transformation matrix of each region and/or subtile for different cycle(s) and/or channel(s) may also be stored in the data storage device 1422.

The user interface 1424 may be used by a user to operate the sequencing system or access data stored in the data storage 1422 or the computer system 1426.

The computer system 1426 may control the general operation of the sequencing system and may be coupled to the user interface 1424. It may also perform steps in operations herein. The computer system 1426 may store information regarding the operation of the sequencing system 1410, such as configuration information, instructions for operating the sequencing system 1410, or user information. The computer system 1426 may be configured to pass information between the sequencing system 1410 and the cloud 1430.

As discussed above, the sequencing system 1410 may have dedicated processors 1418, FPGA(s) 1420, and/or the computer system 1426. The sequencing system may use one, two, or all of these elements to accomplish necessary processing described above. In some embodiments, when these elements are present together, the processing tasks are split between them. For example, the FPGA(s) 1420 may be used to perform some or all of: the preprocessing operations, image registration, and the subsequent operations, while the computer system 1426 may perform other processing functions for the sequencing system 1410 such as base calling. Those skilled in the art will understand that various combinations of these elements will allow various system embodiments that balance efficiency and speed of processing with cost of processing elements.

The cloud 1430 may be a network, remote storage, or some other remote computing system separate from the sequencing system 1410. The connection to cloud 1430 may allow access to data stored externally to the sequencing system 1410 or allow for updating of software in the sequencing system 1410.

The imager 1416 in FIG. 14 can include one or more optical imaging systems. Further disclosed herein are optical imaging system design guidelines and high-performance fluorescence imaging methods and systems that provide improved optical resolution and image quality for fluorescence imaging-based genomics applications. The disclosed optical imaging system designs provide for larger fields-of-view, increased spatial resolution, improved modulation transfer, contrast-to-noise ratio, and image quality, higher spatial sampling frequency, faster transitions between image capture when repositioning the sample plane to capture a series of images (e.g., of different fields-of-view), and improved imaging system duty cycle, and thus enable higher throughput image acquisition and analysis.

In some instances, improvements in imaging performance, e.g., for dual-side (flow cell) imaging applications or other multi-surface imaging applications, may be achieved by using an electro-optical phase plate in combination with an objective lens to compensate for the optical aberrations induced by the layer of fluid separating the upper (near) and lower (far) interior surfaces of a flow cell. In some instances, this design approach may also compensate for vibrations introduced by, e.g., a motion-actuated compensator that is moved in or out of the optical path depending on which surface of the flow cell is being images.

In some instances, improvements in imaging performance, e.g., for dual-side (flow cell) imaging applications or other multiple surface imaging applications comprising the use of thick flow cell walls (e.g., wall (or coverslip) thickness >700 μm) and fluid channels (e.g., fluid channel height or thickness of 50-200 μm) may be achieved even when using commercially-available, off-the-shelf objectives by using a tube lens design that corrects for the optical aberrations induced by the thick flow cell walls and/or intervening fluid layer in combination with the objective.

In some instances, improvements in imaging performance, e.g., for multichannel (e.g., two-color or four-color) imaging applications, may be achieved by using multiple tube lenses, one for each imaging channel, where each tube lens design has been optimized for the specific wavelength range used in that imaging channel. Alternatively, multichannel imaging can be achieved through an acousto-optical beam splitter (AOBS), which uses an optical crystal to provide selective spectral reflection and transmission characteristics.

Exemplary embodiments disclosed herein may comprise fluorescence imaging systems, said systems comprising: a) at least one light source configured to provide excitation light within one or more specified wavelength ranges; b) an objective lens configured to collect fluorescence light arising from within a specified field-of-view of a sample plane upon exposure of the sample plane to the excitation light, wherein a numerical aperture of the objective lens is at least 0.1, at least 0.2, at least 0.3, at least 0.4, at least 0.5, at least 0.6, at least 0.7, at least 0.8, or at least 0.9 or a numerical aperture value falling within a range defined by any two of the foregoing; wherein a working distance of the objective lens is at least 400 μm, at least 500 μm, at least 600 μm, at least 700 μm, at least 800 μm, at least 900 μm, at least 1000 μm, or a working distance falling within a range defined by any two of the foregoing; and wherein the field-of-view has an area of at least 0.1 mm², at least 0.2 mm², at least 0.5 mm², at least 0.7 mm², at least 1 mm², at least 2 mm², at least 3 mm², at least 5 mm², or at least 10 mm², or a field of view falling within a range defined by any two of the foregoing; and c) at least one image sensor, wherein the fluorescence collected by the objective lens is imaged onto the image sensor, and wherein a pixel dimension for the image sensor is chosen such that a spatial sampling frequency for the fluorescence imaging system is at least twice an optical resolution of the fluorescence imaging system.

In some embodiments, the numerical aperture may be at least 0.75. In some embodiments, the numerical aperture is at least 1.0. In some embodiments, the working distance is at least 850 μm. In some embodiments, the working distance is at least 1,000 μm. In some embodiments, the field-of-view may have an area of at least 2.5 mm². In some embodiments, the field-of-view may have an area of at least 3 mm². In some embodiments, the field-of-view may have an area of at least 4 mm². In some embodiments, the field-of-view may have an area of at least 5 mm². In some embodiments, the spatial sampling frequency may be at least 2.5 times the optical resolution of the fluorescence imaging system. In some embodiments, the spatial sampling frequency may be at least 3 times the optical resolution of the fluorescence imaging system. In some embodiments, the system may further comprise an X-Y-Z translation stage such that the system is configured to acquire a series of two or more fluorescence images in an automated fashion, wherein each image of the series is or can be acquired for a different field-of-view. In some embodiments, a position of the sample plane may be simultaneously adjusted in an X direction, a Y direction, and a Z direction to match the position of an objective lens focal plane in between acquiring images for different fields-of-view. In some embodiments, the time required for the simultaneous adjustments in the X direction, Y direction, and Z direction may be less than 0.3 seconds, less than 0.4 seconds, less than 0.5 seconds, less than 0.7 seconds, or less than 1 second, or a time falling within a range defined by any two of the foregoing. In some embodiments, the system further comprises an autofocus mechanism configured to adjust the focal plane position prior to acquiring an image of a different field-of-view if an error signal indicates that a difference in the position of the focal plane and the sample plane in the Z direction is greater than a specified error threshold. In some embodiments, the specified error threshold is 100 nm or greater. In some embodiments, the specified error threshold is 50 nm or less. In some embodiments, the system comprises three or more image sensors, and wherein the system is configured to image fluorescence in each of three or more wavelength ranges onto a different image sensor. In some embodiments, a difference in the position of a focal plane for each of the three or more image sensors and the sample plane is less than 100 nm. In some embodiments, a difference in the position of a focal plane for each of the three or more image sensors and the sample plane is less than 50 nm. In some embodiments, the total time required to reposition the sample plane, adjust focus if necessary, and acquire an image is less than 0.4 seconds per field-of-view. In some embodiments, the total time required to reposition the sample plane, adjust focus if necessary, and acquire an image is less than 0.3 seconds per field-of-view.

Also disclosed herein are fluorescence imaging systems for dual-side or multi-side imaging of a flow cell comprising: a) an objective lens configured to collect fluorescence arising from within a specified field-of-view of a sample plane within the flow cell; and b) at least one tube lens positioned between the objective lens and at least one image sensor, wherein the at least one tube lens is configured to correct an imaging performance metric for a combination of the objective lens, the at least one tube lens, and the at least one image sensor when imaging an interior surface of the flow cell, and wherein the flow cell has a wall thickness of at least 700 μm and a gap between an upper interior surface and a lower interior surface of at least 50 μm; wherein the imaging performance metric is substantially the same for imaging the upper interior surface or the lower interior surface of the flow cell without moving an optical compensator into or out of an optical path between the flow cell and the at least one image sensor, without moving one or more optical elements of the tube lens along the optical path, and without moving one or more optical elements of the tube lens into or out of the optical path.

In some embodiments, the objective lens may be a commercially-available microscope objective. In some embodiments, the commercially-available microscope objective may have a numerical aperture of at least 0.3. In some embodiments, the objective lens may have a working distance of at least 700 μm. In some embodiments, the objective lens may be corrected to compensate for a cover slip thickness (or flow cell wall thickness) of 0.17 mm or of greater or lesser thickness than 0.17 mm. In some embodiments, the optical system may be corrected to compensate for cover slip thickness, flow cell thickness, or distance between desired focal planes. In some embodiments, said correction may be made by inserting a corrective optic, such as a lens or optical assembly into the light path of the optical system. In some embodiments, said correction may be made without inserting a corrective optic, such as a lens or optical assembly into the light path of the optical system. In some embodiments, the fluorescence imaging system may further comprise an electro-optical phase plate positioned adjacent to the objective lens and between the objective lens and the tube lens, wherein the electro-optical phase plate may provide correction for optical aberrations caused by a fluid filling the gap between the upper interior surface and the lower interior surface of the flow cell. In some embodiments, the at least one tube lens may be a compound lens comprising three or more optical components. In some embodiments, the at least one tube lens is a compound lens comprising four optical components, which may comprise one or more of a first asymmetric convex-convex lens, a second convex-plano lens, a third asymmetric concave-concave lens, and a fourth asymmetric convex-concave lens which may be present in the order as listed above, or in any alternate order. In some embodiments, the at least one tube lens is configured to correct an imaging performance metric for a combination of the objective lens, the at least one tube lens, and the at least one image sensor when imaging an interior surface of a flow cell having a wall thickness of at least 1 mm. In some embodiments, the at least one tube lens is configured to correct an imaging performance metric for a combination of the objective lens, the at least one tube lens, and the at least one image sensor when imaging an interior surface of a flow cell having a gap of at least 100 μm. In some embodiments, the at least one tube lens is configured to correct an imaging performance metric for a combination of the objective lens, the at least one tube lens, and the at least one image sensor when imaging an interior surface of a flow cell having a gap of at least 200 μm. In some embodiments, the system comprises a single objective lens, two tube lenses, and two image sensors, and each of the two tube lenses is designed to provide optimal imaging performance at a different fluorescence wavelength. In some embodiments, the system comprises a single objective lens, three tube lenses, and three image sensors, and each of the three tube lenses is designed to provide optimal imaging performance at a different fluorescence wavelength. In some embodiments, the system comprises a single objective lens, four tube lenses, and four image sensors, and each of the four tube lenses is designed to provide optimal imaging performance at a different fluorescence wavelength. In some embodiments, the design of the objective lens or the at least one tube lens is configured to optimize the modulation transfer function in the mid to high spatial frequency range. In some embodiments, the imaging performance metric comprises a measurement of modulation transfer function (MTF) at one or more specified spatial frequencies, defocus, spherical aberration, chromatic aberration, coma, astigmatism, field curvature, image distortion, contrast-to-noise ratio (CNR), or any combination thereof. In some embodiments, the difference in the imaging performance metric for imaging the upper interior surface and the lower interior surface of the flow cell is less than 10%. In some embodiments, the difference in imaging performance metric for imaging the upper interior surface and the lower interior surface of the flow cell is less than 5%. In some embodiments, the use of the at least one tube lens provides for an at least equivalent or better improvement in the imaging performance metric for dual-side or multi-side imaging compared to that for a conventional system comprising an objective lens, a motion-actuated compensator, and an image sensor. In some embodiments, the use of the at least one tube lens provides for an at least 10% improvement in the imaging performance metric for dual-side or multi-side imaging compared to that for a conventional system comprising an objective lens, a motion-actuated compensator, and an image sensor.

Disclosed herein are illumination systems for use in imaging-based solid-phase genotyping and sequencing applications, the illumination system comprising: a) a light source; and b) a liquid light-guide configured to collect light emitted by the light source and deliver it to a specified field-of-illumination on a support surface comprising tethered biological macromolecules.

In some embodiments, the illumination system further comprises a condenser lens. In some embodiments, the specified field-of-illumination has an area of at least 2 mm². In some embodiments, the light delivered to the specified field-of-illumination is of uniform intensity across a specified field-of-view for an imaging system used to acquire images of the support surface. In some embodiments, the specified field-of-view has an area of at least 2 mm². In some embodiments, the light delivered to the specified field-of-illumination is of uniform intensity across the specified field-of-view when a coefficient of variation (CV) for light intensity is less than 10%. In some embodiments, the light delivered to the specified field-of-illumination is of uniform intensity across the specified field-of-view when a coefficient of variation (CV) for light intensity is less than 5%. In some embodiments, the light delivered to the specified field-of-illumination has a speckle contrast value of less than 0.1. In some embodiments, the light delivered to the specified field-of-illumination has a speckle contrast value of less than 0.05.

It will be understood by those of skill in the art that the disclosed optical systems, imaging systems, or modules may, in some instances, be stand-alone optical systems designed for imaging a sample or substrate surface. In some instances, they may comprise one or more processors or computers. In some instances, they may comprise one or more software packages that provide instrument control functionality and/or image processing functionality. In some instances, in addition to optical components such as light sources (e.g., solid-state lasers, dye lasers, diode lasers, arc lamps, tungsten-halogen lamps, etc.), lenses, prisms, mirrors, dichroic reflectors, optical filters, optical bandpass filters, apertures, and image sensors (e.g., complementary metal oxide semiconductor (CMOS) image sensors and cameras, charge-coupled device (CCD) image sensors and cameras, etc.), they may also include mechanical and/or optomechanical components, such as an X-Y translation stage, an X-Y-Z translation stage, a piezoelectric focusing mechanism, and the like. In some instances, they may function as modules, components, sub-assemblies, or sub-systems of larger systems designed for genomics applications (e.g., genetic testing and/or nucleic acid sequencing applications). For example, in some instances, they may function as modules, components, sub-assemblies, or sub-systems of larger systems that further comprise light-tight and/or other environmental control housings, temperature control modules, fluidics control modules, fluid dispensing robotics, pick-and-place robotics, one or more processors or computers, one or more local and/or cloud-based software packages (e.g., instrument/system control software packages, image processing software packages, data analysis software packages), data storage modules, data communication modules (e.g., Bluetooth, WiFi, intranet, or internet communication hardware and associated software), display modules, or any combination thereof.

### Methods for Sequencing

The present disclosure provides methods for sequencing immobilized or non-immobilized template molecules. The methods can be operated in system 1400, for example, in sequencer 1414. In some embodiments, the immobilized template molecules comprise a plurality of nucleic acid template molecules having one copy of a target sequence of interest. In some embodiments, nucleic acid template molecules having one copy of a target sequence of interest can be generated by conducting bridge amplification using linear library molecules. In some embodiments, the immobilized template molecules comprise a plurality of nucleic acid template molecules each having two or more tandem copies of a target sequence of interest (e.g., concatemers). In some embodiments, nucleic acid template molecules comprising concatemer molecules can be generated by conducting rolling circle amplification of circularized linear library molecules. In some embodiments, the non-immobilized template molecules comprise circular molecules. In some embodiments, methods for sequencing employ soluble (e.g., non-immobilized) sequencing polymerases or sequencing polymerases that are immobilized to a support.

In some embodiments, the sequencing reactions employ detectably labeled nucleotide analogs. In some embodiments, the sequencing reactions employ a two-stage sequencing reaction comprising binding detectably labeled multivalent molecules, and incorporating nucleotide analogs. In some embodiments, the sequencing reactions employ non-labeled nucleotide analogs. In some embodiments, the sequencing reactions employ phosphate chain labeled nucleotides.

### Supports and Low Non-Specific Coatings

In some embodiments, the methods for sequencing described herein comprise supports, and flow cells comprising supports, which are compatible with the optical test targets, flow cell devices, and methods of employing same described supra.

In some embodiments, the flow cell 1412 in FIG. 14 can include a support, e.g., a solid support as disclosed herein. The present disclosure provides pairwise sequencing compositions and methods which employ a support comprising a plurality of oligonucleotide surface primers immobilized thereon. In some embodiments, the support is passivated with a low non-specific binding coating. The surface coatings described herein can exhibit very low non-specific binding to reagents typically used for nucleic acid capture, amplification and sequencing workflows, such as dyes, nucleotides, enzymes, and nucleic acid primers. The surface coatings exhibit low background fluorescence signals or high contrast-to-noise (CNR) ratios compared to conventional surface coatings.

The low non-specific binding coating comprises one layer or multiple layers. In some embodiments, the plurality of surface primers are immobilized to the low non-specific binding coating. In some embodiments, at least one surface primer is embedded within the low non-specific binding coating. The low non-specific binding coating enables improved nucleic acid hybridization and amplification performance. In general, the supports comprise a substrate (or support structure), one or more layers of a covalently or non-covalently attached low-binding, chemical modification layers, e.g., silane layers, polymer films, and one or more covalently or non-covalently attached surface primers that can be used for tethering single-stranded nucleic acid library molecules to the support. In some embodiments, the formulation of the coating, e.g., the chemical composition of one or more layers, the coupling chemistry used to cross-link the one or more layers to the support and/or to each other, and the total number of layers, may be varied such that non-specific binding of proteins, nucleic acid molecules, and other hybridization and amplification reaction components to the coating is minimized or reduced relative to a comparable monolayer. The formulation of the coating described herein may be varied such that non-specific hybridization on the coating is minimized or reduced relative to a comparable monolayer. The formulation of the coating may be varied such that non-specific amplification on the coating is minimized or reduced relative to a comparable monolayer. The formulation of the coating may be varied such that specific amplification rates and/or yields on the coating are maximized. Amplification levels suitable for detection are achieved in no more than 2, 3, 4, 5, 6, 7, 8, 9, 10, 15, 20, 25, 30, or more than 30 amplification cycles in some cases disclosed herein.

The support structure that comprises the one or more chemically-modified layers, e.g., layers of a low non-specific binding polymer, may be independent or integrated into another structure or assembly. For example, in some embodiments, the support structure may comprise one or more surfaces within an integrated or assembled microfluidic flow cell. The support structure may comprise one or more surfaces within a microplate format, e.g., the bottom surface of the wells in a microplate. In some embodiments, the support structure comprises the interior surface (such as the lumen surface) of a capillary. In some embodiments, the support structure comprises the interior surface (such as the lumen surface) of a capillary etched into a planar chip.

The attachment chemistry used to graft a first chemically-modified layer to the surface of the support will generally be dependent on both the material from which the surface is fabricated and the chemical nature of the layer. In some embodiments, the first layer may be covalently attached to the surface. In some embodiments, the first layer may be non-covalently attached, e.g., adsorbed to the support through non-covalent interactions such as electrostatic interactions, hydrogen bonding, or van der Waals interactions between the support and the molecular components of the first layer. In either case, the support may be treated prior to attachment or deposition of the first layer. Any of a variety of surface preparation techniques known to those of skill in the art may be used to clean or treat the surface. For example, glass or silicon surfaces may be acid-washed using a Piranha solution (a mixture of sulfuric acid (H₂SO₄) and hydrogen peroxide (H₂O₂)), base treatment in KOH and NaOH, and/or cleaned using an oxygen plasma treatment method.

Silane chemistries constitute non-limiting approaches for covalently modifying the silanol groups on glass or silicon surfaces to attach more reactive functional groups (e.g., amines, aldehydes, epoxy or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding coatings include, but are not limited to, (3-Aminopropyl) trimethoxysilane (APTMS), (3-Aminopropyl)triethoxysilane (APTES), any of a variety of PEG-silanes (e.g., comprising molecular weights of 1K, 2K, 5K, 10K, 20K, etc.), amino-PEG silane (i.e., comprising a free amino functional group), maleimide-PEG silane, biotin-PEG silane, and the like.

Any of a variety of molecules known to those of skill in the art including, but not limited to, amino acids, peptides, nucleotides, oligonucleotides, other monomers or polymers, or combinations thereof may be used in creating the one or more chemically-modified layers on the support, wherein the choice of components used may be varied to alter one or more properties of the layers, e.g., the surface density of functional groups and/or tethered oligonucleotide primers, the hydrophilicity/hydrophobicity of the layers, or the three three-dimensional nature (i.e., “thickness”) of the layer. Examples of polymers that may be used to create one or more layers of low non-specific binding material in any of the disclosed coatings include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface and/or to cross-link the layers to each other include, but are not limited to, biotin-streptavidin interactions (or variations thereof), his tag—Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

The low non-specific binding surface coating may be applied uniformly across the support. Alternatively, the surface coating may be patterned, such that the chemical modification layers are confined to one or more discrete regions of the support. For example, the coating may be patterned using photolithographic techniques to create an ordered array or random pattern of chemically-modified regions on the support. Alternately or in combination, the coating may be patterned using, e.g., contact printing and/or ink-jet printing techniques. In some embodiments, an ordered array or random pattern of chemically-modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In some embodiments, the low nonspecific binding coatings comprise hydrophilic polymers that are non-specifically adsorbed or covalently grafted to the support. Typically, passivation is performed utilizing poly(ethylene glycol) (PEG, also known as polyethylene oxide (PEO) or polyoxyethylene) or other hydrophilic polymers with different molecular weights and end groups that are linked to a support using, for example, silane chemistry. The end groups distal from the surface can include, but are not limited to, biotin, methoxy ether, carboxylate, amine, NHS ester, maleimide, and bis-silane. In some embodiments, two or more layers of a hydrophilic polymer, e.g., a linear polymer, branched polymer, or multi-branched polymer, may be deposited on the surface. In some embodiments, two or more layers may be covalently coupled to each other or internally cross-linked to improve the stability of the resulting coating. In some embodiments, surface primers with different nucleotide sequences and/or base modifications (or other biomolecules, e.g., enzymes or antibodies) may be tethered to the resulting layer at various surface densities. In some embodiments, for example, both surface functional group density and surface primer concentration may be varied to attain a desired surface primer density range. Additionally, surface primer density can be controlled by diluting the surface primers with other molecules that carry the same functional group. For example, amine-labeled surface primers can be diluted with amine-labeled polyethylene glycol in a reaction with an NHS-ester coated surface to reduce the final primer density. Surface primers with different lengths of linker between the hybridization region and the surface attachment functional group can also be applied to control surface density. Example of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of known concentration.

In some embodiments, the low nonspecific binding coatings comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl) acrylamide-co-acrylamide (PAZAM).

In order to scale primer surface density and add additional dimensionality to hydrophilic or amphoteric coatings, supports comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the support significantly. Traditional PEG coating approaches use monolayer primer deposition, which have been generally reported for single molecule applications, but do not yield high copy numbers for nucleic acid amplification applications. As described herein “layering” can be accomplished using traditional crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some embodiments, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some embodiments, high primer density materials may be constructed in solution and subsequently layered onto the surface in multiple steps.

Examples of materials from which the support structure may be fabricated include, but are not limited to, glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic support structures are contemplated.

The support structure may be rendered in any of a variety of geometries and dimensions known to those of skill in the art, and may comprise any of a variety of materials known to those of skill in the art. For example, the support structure may be locally planar or flat (e.g., comprising a microscope slide or the surface of a microscope slide). Globally, the support structure may be cylindrical (e.g., comprising a capillary or the interior surface of a capillary), spherical (e.g., comprising the outer surface of a non-porous bead), or irregular (e.g., comprising the outer surface of an irregularly-shaped, non-porous bead or particle). For example, the substrate(s) disclosed herein of the solid optical test target may be planar or flat. Alternatively, the substrate(s) may have other non-flat or curved shapes, e.g., cylindrical or irregular. In some embodiments, the surface of the support structure used for nucleic acid hybridization and amplification may be a solid, non-porous surface. In some embodiments, the surface of the support structure used for nucleic acid hybridization and amplification may be porous, such that the coatings described herein penetrate the porous surface, and nucleic acid hybridization and amplification reactions performed thereon may occur within the pores.

The support structure that comprises the one or more chemically-modified layers, e.g., layers of a low non-specific binding polymer, may be independent or integrated into another structure or assembly. For example, the support structure may comprise one or more surfaces within an integrated or assembled microfluidic flow cell. The support structure may comprise one or more surfaces within a microplate format, e.g., the bottom surface of the wells in a microplate. In some embodiments, the support structure comprises the interior surface (such as the lumen surface) of a capillary. In some embodiments the support structure comprises the interior surface (such as the lumen surface) of a capillary etched into a planar chip.

As noted, the low non-specific binding supports of the present disclosure exhibit reduced non-specific binding of proteins, nucleic acids, and other components of the hybridization and/or amplification formulation used for solid-phase nucleic acid amplification. The degree of non-specific binding exhibited by a given support surface may be assessed either qualitatively or quantitatively. For example, exposure of the surface to fluorescent dyes (e.g., cyanins such as Cy3, or Cy5, etc., fluoresceins, coumarins, rhodamines, etc. or other dyes disclosed herein), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a qualitative tool for comparison of non-specific binding on supports comprising different surface formulations. In some embodiments, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on supports comprising different surface formulations-provided that care has been taken to ensure that the fluorescence imaging is performed under conditions where fluorescence signal is linearly related (or related in a predictable manner) to the number of fluorophores on the support surface (e.g., under conditions where signal saturation and/or self-quenching of the fluorophore is not an issue) and suitable calibration standards are used. In some embodiments, other techniques known to those of skill in the art, for example, radioisotope labeling and counting methods may be used for quantitative assessment of the degree to which non-specific binding is exhibited by the different support surface formulations of the present disclosure.

Some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed be detection of the amount of label remaining on the surface and comparison of the signal resulting therefrom to an appropriate calibration standard. In some embodiments, the label may comprise a fluorescent label. In some embodiments, the label may comprise a radioisotope. In some embodiments, the label may comprise any other detectable label known to one of skill in the art. In some embodiments, the degree of non-specific binding exhibited by a given support surface formulation may thus be assessed in terms of the number of non-specifically bound protein molecules (or nucleic acid molecules or other molecules) per unit area. In some embodiments, the low-binding supports of the present disclosure may exhibit non-specific protein binding (or non-specific binding of other specified molecules, (e.g., cyanins such as Cy3, or Cy5, etc., fluoresceins, coumarins, rhodamines, etc. or other dyes disclosed herein)) of less than 0.001 molecule per μm², less than 0.01 molecule per μm², less than 0.1 molecule per μm², less than 0.25 molecule per μm², less than 0.5 molecule per μm², less than 1 molecule per μm², less than 10 molecules per μm², less than 100 molecules per μm², or less than 1,000 molecules per μm². Those of skill in the art will realize that a given support surface of the present disclosure may exhibit non-specific binding falling anywhere within this range, for example, of less than 86 molecules per μm². For example, some modified surfaces disclosed herein exhibit nonspecific protein binding of less than 0.5 molecule/μm² following contact with a 1 μM solution of Cy3 labeled streptavidin (GE Amersham) in phosphate buffered saline (PBS) buffer for 15 minutes, followed by 3 rinses with deionized water. Some modified surfaces disclosed herein exhibit nonspecific binding of Cy3 dye molecules of less than 0.25 molecules per μm². In independent nonspecific binding assays, 1 μM labeled Cy3 SA (ThermoFisher), 1 μM Cy5 SA dye (ThermoFisher), 10 μM Aminoallyl-dUTP-ATTO-647N (Jena Biosciences), 10 μM Aminoallyl-dUTP-ATTO-Rhol 1 (Jena Biosciences), 10 μM Aminoallyl-dUTP-ATTO-Rhol 1 (Jena Biosciences), 10 μM 7-Propargylamino-7-deaza-dGTP-Cy5 (Jena Biosciences, and 10 μM 7-Propargylamino-7-deaza-dGTP-Cy3 (Jena Biosciences) were incubated on the low binding coated supports at 37° C. for 15 minutes in a 384 well plate format. Each well was rinsed 2-3× with 50 μl deionized RNase/DNase Free water and 2-3× with 25 mM ACES buffer pH 7.4. The 384 well plates were imaged on a GE Typhoon instrument using the Cy3, AF555, or Cy5 filter sets (according to dye test performed) as specified by the manufacturer at a PMT gain setting of 800 and resolution of 50-100 μm. For higher resolution imaging, images were collected on an Olympus IX83 microscope (e.g., inverted fluorescence microscope) (Olympus Corp., Center Valley, Pa.) with a total internal reflectance fluorescence (TIRF) objective (100×, 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. Dichroic mirrors were purchased from Semrock (IDEX Health & Science, LLC, Rochester, N.Y.), e.g., 405, 488, 532, or 633 nm dichroic reflectors/beamsplitters, and band pass filters were chosen as 532 LP or 645 LP concordant with the appropriate excitation wavelength. Some modified surfaces disclosed herein exhibit nonspecific binding of dye molecules of less than 0.25 molecules per μm². In some embodiments, the coated support was immersed in a buffer (e.g., 25 mM ACES, pH 7.4) while the image was acquired.

In some embodiments, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some embodiments, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some embodiments, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

In some embodiments, the hydrophilic surfaces disclosed herein facilitate reduced wash times for bioassays, often due to reduced nonspecific binding of biomolecules to the low-binding surfaces. In some embodiments, adequate wash steps may be performed in less than 60, 50, 40, 30, 20, 15, 10, or less than 10 seconds. For example, adequate wash steps may be performed in less than 30 seconds.

Some low-binding surfaces of the present disclosure exhibit significant improvement in stability or durability to prolonged exposure to solvents and elevated temperatures, or to repeated cycles of solvent exposure or changes in temperature. For example, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some embodiments, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some embodiments, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some embodiments, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100 fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100 fold greater than a signal of an adjacent amplified nucleic acid population region of the surface.

In some embodiments, fluorescence images of the disclosed low background surfaces when used in nucleic acid hybridization or amplification applications to create polonies of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

One or more types of primer may be attached or tethered to the support surface. In some embodiments, the one or more types of adapters or primers may comprise spacer sequences, adapter sequences for hybridization to adapter-ligated target library nucleic acid sequences, forward amplification primers, reverse amplification primers, sequencing primers, and/or molecular barcoding sequences, or any combination thereof. In some embodiments, 1 primer or adapter sequence may be tethered to at least one layer of the surface. In some embodiments, at least 2, 3, 4, 5, 6, 7, 8, 9, 10, or more than 10 different primer or adapter sequences may be tethered to at least one layer of the surface.

In some embodiments, the tethered adapter and/or primer sequences may range in length from about 10 nucleotides to about 100 nucleotides. In some embodiments, the tethered adapter and/or primer sequences may be at least 10, at least 20, at least 30, at least 40, at least 50, at least 60, at least 70, at least 80, at least 90, or at least 100 nucleotides in length. In some embodiments, the tethered adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some embodiments the length of the tethered adapter and/or primer sequences may range from about 20 nucleotides to about 80 nucleotides. Those of skill in the art will recognize that the length of the tethered adapter and/or primer sequences may have any value within this range, e.g., about 24 nucleotides.

In some embodiments, the resultant surface density of primers (e.g., capture primers) on the low binding support surfaces of the present disclosure may range from about 100 primer molecules per μm² to about 100,000 primer molecules per μm². In some embodiments, the resultant surface density of primers on the low binding support surfaces of the present disclosure may range from about 1,000 primer molecules per μm² to about 1,000,000 primer molecules per μm². In some embodiments, the surface density of primers may be at least 1,000, at least 10,000, at least 100,000, or at least 1,000,000 molecules per μm². In some embodiments, the surface density of primers may be at most 1,000,000, at most 100,000, at most 10,000, or at most 1,000 molecules per μm². Any of the lower and upper values described in this paragraph may be combined to form a range included within the present disclosure, for example, in some embodiments the surface density of primers may range from about 10,000 molecules per μm² to about 100,000 molecules per μm². Those of skill in the art will recognize that the surface density of primer molecules may have any value within this range, e.g., about 455,000 molecules per μm². In some embodiments, the surface density of target library nucleic acid sequences initially hybridized to adapter or primer sequences on the support surface may be less than or equal to that indicated for the surface density of tethered primers. In some embodiments, the surface density of clonally-amplified target library nucleic acid sequences hybridized to adapter or primer sequences on the support surface may span the same range as that indicated for the surface density of tethered primers.

Local densities as listed above do not preclude variation in density across a surface, such that a surface may comprise a region having an oligo density of, for example, 500,000/μm², while also comprising at least a second region having a substantially different local density.

In some embodiments, the support is solid, semi-solid, or a combination of both. In some embodiments, the support is porous, semi-porous, non-porous, or any combination of porosity. In some embodiments, the support can be substantially planar, concave, convex, or any combination thereof. In some embodiments, the support can be cylindrical, for example comprising a capillary or interior surface of a capillary.

In some embodiments, the surface of the support can be substantially smooth. In some embodiments, the support can be regularly or irregularly textured, including bumps, etched, pores, three-dimensional scaffolds, or any combination thereof.

In some embodiments, the support comprises a bead having any shape, including spherical, hemi-spherical, cylindrical, barrel-shaped, toroidal, disc-shaped, rod-like, conical, triangular, cubical, polygonal, tubular or wire-like.

The support can be fabricated from any material, including but not limited to glass, plastic, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

In some embodiments, the surface of the support is coated with one or more compounds to produce a passivated layer on the support. In these embodiments, the properties of the passivated layer are taken into account in the hypothetical flow cell employed by the optical test targets described herein. In some embodiments, the support comprises a low non-specific binding surface that enable improved nucleic acid hybridization and amplification performance on the support. In general, the support may comprise one or more layers of a covalently or non-covalently attached low-binding, chemical modification layers, e.g., silane layers, polymer films, and one or more covalently or non-covalently attached oligonucleotides that may be used for immobilizing a plurality of nucleic acid template molecules to the support, all of which can be taken into account in determining the properties of the hypothetical flow cell.

In some embodiments, the degree of hydrophilicity (or “wettability” with aqueous solutions) of the surface coatings may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

The present disclosure provides a plurality (e.g., two or more) of nucleic acid templates immobilized to a support. In some embodiments, the immobilized plurality of nucleic acid templates have the same sequence or have different sequences. In some embodiments, individual nucleic acid template molecules in the plurality of nucleic acid templates are immobilized to a different site on the support. In some embodiments, two or more individual nucleic acid template molecules in the plurality of nucleic acid templates are immobilized to a site on the support. In some embodiments, the support comprises a plurality of sites arranged in an array. The term “array” refers to a support comprising a plurality of sites located at pre-determined locations on the support to form an array of sites. The sites can be discrete and separated by interstitial regions. In some embodiments, the pre-determined sites on the support can be arranged in one dimension in a row or a column, or arranged in two dimensions in rows and columns. In some embodiments, the plurality of pre-determined sites is arranged on the support in an organized fashion. In some embodiments, the plurality of pre-determined sites is arranged in any organized pattern, including rectilinear, hexagonal patterns, grid patterns, patterns having reflective symmetry, patterns having rotational symmetry, or the like. The pitch between different pairs of sites can be that same or can vary. In some embodiments, the support can have nucleic acid template molecules immobilized at a plurality of sites at a surface density of about 10²-10¹⁵ sites per mm², or more, to form a nucleic acid template array. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, wherein the sites are located at pre-determined locations on the support. In some embodiments, a plurality of pre-determined sites on the support (e.g., 10²-10¹⁵ sites or more) comprise immobilized with nucleic acid templates to form a nucleic acid template array. In some embodiments, the nucleic acid templates are immobilized at a plurality of pre-determined sites by hybridization to immobilized surface capture primers, or the nucleic acid templates are covalently attached to the surface capture primers. In some embodiments, the nucleic acid templates are immobilized at a plurality of pre-determined sites, for example immobilized at 10²-10¹⁵ sites or more. In some embodiments, the nucleic acid templates that are immobilized at a plurality of sites on the support comprise linear or circular nucleic acid template molecules, or a mixture of both linear and circular molecules. In some embodiments, the immobilized nucleic acid templates are clonally-amplified to generate immobilized nucleic acid polonies at the plurality of pre-determined sites. In some embodiments, individual immobilized nucleic acid template molecules comprise one copy of a target sequence of interest, or comprise concatemers having two or more tandem copies of a target sequence of interest.

In some embodiments, a support comprising a plurality of sites located at random locations on the support is referred to herein as a support having randomly located sites thereon. The location of the randomly located sites on the support are not pre-determined. The plurality of randomly-located sites is arranged on the support in a disordered and/or unpredictable fashion, although the skilled artisan will appreciate that the located sites can be not predetermined, without being classically random. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, wherein the sites are randomly located on the support, or wherein the location of the sites is not pre-determined. In some embodiments, a plurality of randomly located sites on the support (e.g., 10²-10¹⁵ sites or more) are immobilized with nucleic acid templates to form a support immobilized with nucleic acid templates. In some embodiments, the nucleic acid templates that are immobilized at a plurality of randomly located sites by hybridization to immobilized surface capture primers, or the nucleic acid templates are covalently attached to the surface capture primer. In some embodiments, the nucleic acid templates that are immobilized at a plurality of randomly located sites, for example immobilized at 10²-10¹⁵ sites or more. In some embodiments, the nucleic acid templates that are immobilized at a plurality of sites on the support comprise linear or circular nucleic acid template molecules or a mixture of both linear and circular molecules. In some embodiments, the immobilized nucleic acid templates are clonally-amplified to generate immobilized nucleic acid polonies at the plurality of randomly located sites. In some embodiments, individual immobilized nucleic acid template molecules comprise one copy of a target sequence of interest, or comprise concatemers having two or more tandem copies of a target sequence of interest.

In some embodiments, with respect to nucleic acid template molecules immobilized to pre-determined or random sites on the support, the plurality of immobilized nucleic acid template molecules on the support are in fluid communication with each other to permit flowing a solution of reagents (e.g., enzymes including polymerases, multivalent molecules, nucleotides, divalent cations and/or buffers and the like) onto the support so that the plurality of immobilized nucleic acid template molecules on the support can be reacted with the reagents in a massively parallel manner. For example, when the support is an interior surface of a substrate of a flow cell, fluid communication is achieved by flowing solutions of reagents through the flow cell. In some embodiments, the fluid communication of the plurality of immobilized nucleic acid template molecules can be used to conduct nucleotide binding assays and/or conduct nucleotide polymerization reactions (e.g., primer extension or sequencing) on the plurality of immobilized nucleic acid template molecules, and to conduct detection and imaging for massively parallel sequencing. In some embodiments, the term “immobilized” and related terms refer to nucleic acid molecules or enzymes (e.g., polymerases) that are attached to the support at pre-determined or random locations, wherein the nucleic acid molecules or enzymes are attached directly to a support through covalent bond or non-covalent interaction, or the nucleic acid molecules or enzymes are attached to a coating on the support.

When used in reference to a low binding surface coating, one or more layers of a multi-layered surface coating may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branched PHEMA), branched poly(oligo (ethylene glycol) methyl ether methacrylate) (branched POEGMA), branched polyglutamic acid (branched PGA), branched poly-lysine, branched poly-glucoside, and dextran.

In some embodiments, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branches.

Linear, branched, or multi-branched polymers used to create one or more layers of any of the multi-layered surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some embodiments, e.g., wherein at least one layer of a multi-layered surface comprises a branched polymer, the number of covalent bonds between a branched polymer molecule of the layer being deposited and molecules of the previous layer may range from about one covalent linkage per molecule and about 32 covalent linkages per molecule. In some embodiments, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 covalent linkages per molecule.

Any reactive functional groups that remain following the coupling of a material layer to the surface may optionally be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface, may range from 1 to about 10. In some embodiments, the number of layers is at least 1, at least 2, at least 3, at least 4, at least 5, at least 6, at least 7, at least 8, at least 9, or at least 10. In some embodiments, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some embodiments the number of layers may range from about 2 to about 4. In some embodiments, all of the layers may comprise the same material. In some embodiments, each layer may comprise a different material. In some embodiments, the plurality of layers may comprise a plurality of materials. In some embodiments at least one layer may comprise a branched polymer. In some embodiment, all of the layers may comprise a branched polymer.

One or more layers of low non-specific binding material may in some cases be deposited on and/or conjugated to the substrate surface using a polar protic solvent, a polar or polar aprotic solvent, a nonpolar solvent, or any combination thereof. In some embodiments the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino) propanesulfonic acid (MOPS), etc.), or any combination thereof. In some embodiments, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of water or an aqueous buffer solution. In some embodiments, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of an organic solvent. The pH of the solvent mixture used may be less than 6, about 6, 6.5, 7, 7.5, 8, 8.5, 9, or greater than pH 9.

### Sequencing Libraries

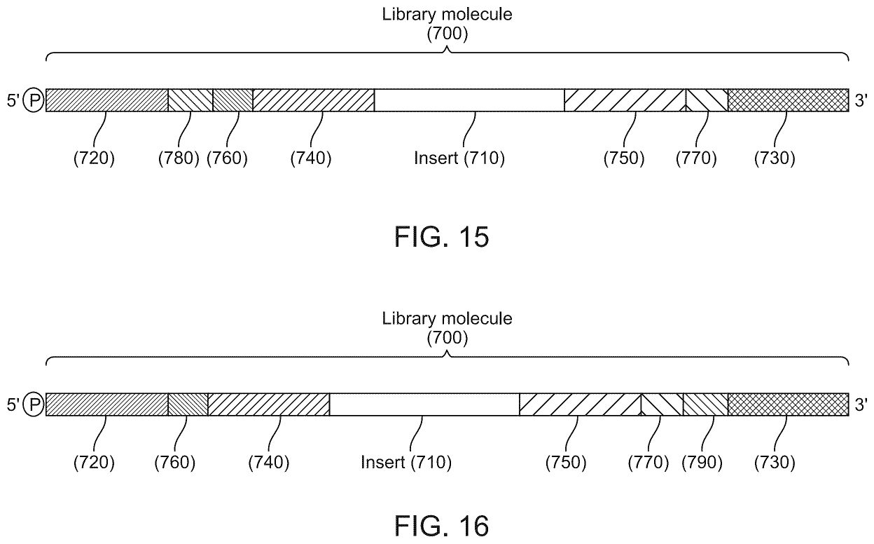

The disclosure provides a plurality of nucleic acid template molecules immobilized to a support, as described herein. In some embodiments, the plurality of nucleic acid template molecules comprise concatemers. In some embodiments, the concatemers comprise tandem repeat units of a sequence-of-interest (e.g., insert region, also sometimes referred to as a template or target sequence) and any adaptor sequences. For example, the tandem repeat unit comprises: (i) a left universal adaptor sequence having a binding sequence for a first surface primer (720, see FIGS. 15 and 16) (e.g., a surface pinning primer), (ii) a left universal adaptor sequence having a binding sequence for a first sequencing primer (740) (e.g., a forward sequencing primer), (iii) a sequence-of-interest (710), (iv) a right universal adaptor sequence having a binding sequence for a second sequencing primer (750) (e.g., reverse sequencing primer), (v) a right universal adaptor sequence having a binding sequence for a second surface primer (730) (e.g., surface capture primer), (vii) a left sample index sequence (760) and/or a right sample index sequence (770). In some embodiments, the tandem repeat unit further comprises a left unique identification sequence (780) and/or a right unique identification sequence (790). In some embodiments, the tandem repeat unit further comprises at least one binding sequence for a compaction oligonucleotide. In some embodiments, FIGS. 15 and 16 show linear library molecules or a unit of a concatemer molecule.

The immobilized concatemer can self-collapse into a compact nucleic acid (nanoball). Inclusion of one or more compaction oligonucleotides during the rolling circle amplification (RCA) reaction that generates the concatemer can further compact the size and/or shape of the nanoball. An increase in the number of tandem repeat units in a given concatemer increases the number of sites along the concatemer for hybridizing to multiple sequencing primers (e.g., sequencing primers having a universal sequence) which serve as multiple initiation sites for polymerase-catalyzed sequencing reactions. When the sequencing reaction employs detectably labeled nucleotides and/or detectably labeled multivalent molecules (e.g., having nucleotide units), the signals emitted by the nucleotides or nucleotide units that participate in the parallel sequencing reactions along the concatemer yields an increased signal intensity for each concatemer. Multiple portions of a given concatemer can be simultaneously sequenced. Furthermore, a plurality of binding complexes can form along a particular concatemer molecule, each binding complex comprising a sequencing polymerase bound to a template/primer duplex and bound to a multivalent molecule, wherein the plurality of binding complexes remain stable without dissociation resulting in increased persistence time which increases signal intensity and reduces imaging time.

### Methods for Sequencing Using Nucleotide Analogs

The present disclosure provides methods for sequencing any of the immobilized template molecules described herein, the methods comprising step (a): contacting a sequencing polymerase with (i) a nucleic acid template molecule and (ii) a nucleic acid sequencing primer, wherein the contacting is conducted under conditions suitable to bind the sequencing polymerase to the nucleic acid template molecule which is hybridized to the nucleic acid primer, wherein the nucleic acid template molecule hybridized to the nucleic acid primer forms the nucleic acid duplex. In some embodiments, the sequencing polymerase comprises a recombinant mutant sequencing polymerase that can bind and incorporate nucleotide analogs.

In some embodiments of the methods for sequencing template molecules, the sequencing primer comprises a 3′ extendible end or a 3′ non-extendible end. In some embodiments, the plurality of nucleic acid template molecules comprise amplified template molecules (e.g., clonally amplified template molecules). In some embodiments, the plurality of nucleic acid template molecules comprise one copy of a target sequence of interest. In some embodiments, the plurality of nucleic acid molecules comprise two or more tandem copies of a target sequence of interest (e.g., concatemers). In some embodiments, the plurality of nucleic acid template molecules comprise the same target sequence of interest or different target sequences of interest. In some embodiments, the plurality of nucleic acid primers are in solution or are immobilized to a support. In some embodiments, when the plurality of nucleic acid template molecules and/or the plurality of nucleic acid primers are immobilized to a support, the binding with the first sequencing polymerase generates a plurality of immobilized first complexed polymerases. In some embodiments, the plurality of nucleic acid template molecules and/or nucleic acid primers are immobilized to 10²-10¹⁵ different sites on a support. In some embodiments, the binding of the plurality of template molecules and nucleic acid primers with the plurality of first sequencing polymerases generates a plurality of first complexed polymerases immobilized to 10²-10¹⁵ different sites on the support. In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

In some embodiments, the methods for sequencing further comprise step (b): contacting the sequencing polymerase with a plurality of nucleotides under a condition suitable for binding at least one nucleotide to the sequencing polymerase which is bound to the nucleic acid duplex and suitable for polymerase-catalyzed nucleotide incorporation which extends the sequencing primer by one nucleotide. In some embodiments, the sequencing polymerase is contacted with the plurality of nucleotides in the presence of at least one catalytic cation comprising magnesium and/or manganese. In some embodiments, the plurality of nucleotides comprises at least one nucleotide analog having a chain terminating moiety at the sugar 2′ or 3′ position. In some embodiments, the chain terminating moiety is removable from the sugar 2′ or 3′ position to convert the chain terminating moiety to an OH or H group. In some embodiments, the plurality of nucleotides comprises at least one nucleotide that lacks a chain terminating moiety. In some embodiments, at least on nucleotide is labeled with a detectable reporter moiety (e.g., fluorophore) that emits a detectable signal. In some embodiments, the detectable reporter moiety comprises a fluorophore. In some embodiments, the fluorophore is attached to the nucleo-base. In some embodiments, the fluorophore is attached to the nucleo-base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleo-base. When the incorporated chain terminating nucleotide is detectably labeled, step (b) further comprises detecting the emitted signal from the incorporated chain terminating nucleotide. In some embodiments, step (b) further comprises identifying the nucleo-based of the incorporated chain terminating nucleotide.

In some embodiments, the methods for sequencing further comprise step (c): removing the chain terminating moiety from the incorporated chain terminating nucleotide to generate an extendible 3′OH group. In some embodiments, step (c) further comprises removing the detectable label from the incorporated chain terminating nucleotide. In some embodiments, the sequencing polymerase remains bound to the template molecule which is hybridized to the sequencing primer which is extended by one nucleo-base.

In some embodiments, the methods for sequencing further comprise step (d): repeating steps (b) and (c) at least once.

### Two-Stage Methods for Nucleic Acid Sequencing

The present disclosure provides a two-stage method for sequencing any of the immobilized template molecules described herein. In some embodiments, the first stage generally comprises binding multivalent molecules to complexed polymerases to form multivalent-complexed polymerases, and detecting the multivalent-complexed polymerases.

In some embodiments, the first stage comprises step (a): contacting a plurality of first sequencing polymerases with (i) a plurality of nucleic acid template molecules and (ii) a plurality of nucleic acid sequencing primers, wherein the contacting is conducted under conditions suitable to bind the plurality of first sequencing polymerases to the plurality of nucleic acid template molecules and the plurality of nucleic acid primers, thereby forming a plurality of first complexed polymerases comprising a first sequencing polymerase bound to a nucleic acid duplex, wherein the nucleic acid duplex comprises a nucleic acid template molecule hybridized to a nucleic acid primer. In some embodiments, the first polymerase comprises a recombinant mutant sequencing polymerase.

In some embodiments, in the methods for sequencing template molecules, the sequencing primer comprises an oligonucleotide having a 3′ extendible end or a 3′ non-extendible end. In some embodiments, the plurality of nucleic acid template molecules comprise amplified template molecules (e.g., clonally amplified template molecules). In some embodiments, the plurality of nucleic acid template molecules comprise one copy of a target sequence of interest. In some embodiments, the plurality of nucleic acid molecules comprise two or more tandem copies of a target sequence of interest (e.g., concatemers). In some embodiments, the nucleic acid template molecules in the plurality of nucleic acid template molecules comprise the same target sequence of interest or different target sequences of interest. In some embodiments, the plurality of nucleic acid template molecules and/or the plurality of nucleic acid primers are in solution or are immobilized to a support. In some embodiments, when the plurality of nucleic acid template molecules and/or the plurality of nucleic acid primers are immobilized to a support, the binding with the first sequencing polymerase generates a plurality of immobilized first complexed polymerases. In some embodiments, the plurality of nucleic acid template molecules and/or nucleic acid primers are immobilized to 10²-10¹⁵ different sites on a support. In some embodiments, the binding of the plurality of template molecules and nucleic acid primers with the plurality of first sequencing polymerases generates a plurality of first complexed polymerases immobilized to 10²-10¹⁵ different sites on the support. In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

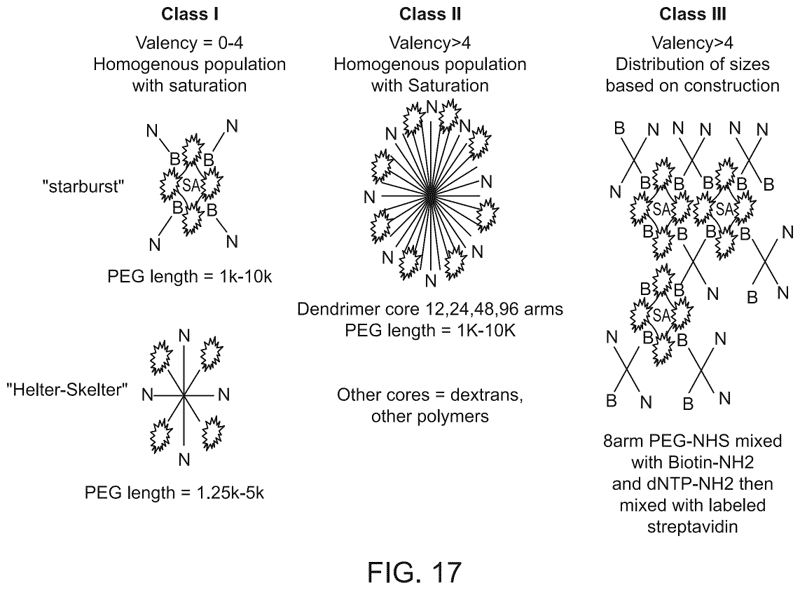

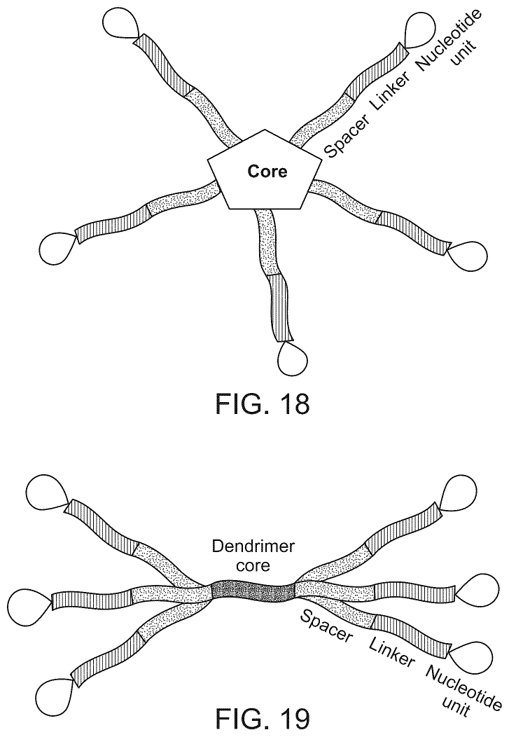

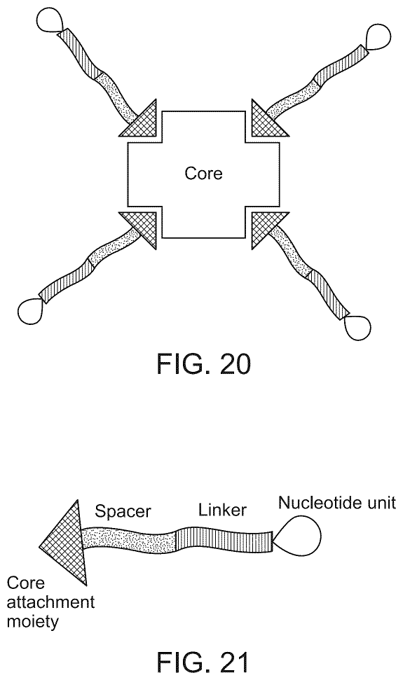

In some embodiments, the methods for sequencing further comprise step (b): contacting the plurality of first complexed polymerases with a plurality of multivalent molecules to form a plurality of multivalent-complexed polymerases (e.g., binding complexes). In some embodiments, individual multivalent molecules in the plurality of multivalent molecules comprise a core attached to multiple nucleotide arms and each nucleotide arm is attached to a nucleotide (e.g., nucleotide unit) (e.g., FIGS. 17-21). In some embodiments, the contacting of step (b) is conducted under conditions suitable for binding complementary nucleotide units of the multivalent molecules to at least two of the plurality of first complexed polymerases, thereby forming a plurality of multivalent-complexed polymerases. In some embodiments, the conditions are suitable for inhibiting polymerase-catalyzed incorporation of the complementary nucleotide units into the primers of the plurality of multivalent-complexed polymerases. In some embodiments, the plurality of multivalent molecules comprise at least one multivalent molecule having multiple nucleotide arms (e.g., FIGS. 17-21) each attached with a nucleotide analog (e.g., nucleotide analog unit), wherein the nucleotide analog includes a chain terminating moiety at the sugar 2′ and/or 3′ position. In some embodiments, the plurality of multivalent molecules comprises at least one multivalent molecule comprising multiple nucleotide arms each attached with a nucleotide unit that lacks a chain terminating moiety. In some embodiments, at least one of the multivalent molecules in the plurality of multivalent molecules is labeled with a detectable reporter moiety that emits a signal. In some embodiments, the detectable reporter moiety comprises a fluorophore. In some embodiments, the contacting of step (b) is conducted in the presence of at least one non-catalytic cation comprising strontium, barium and/or calcium.

In some embodiments, the methods for sequencing further comprise step (c): detecting the plurality of multivalent-complexed polymerases. In some embodiments, the detecting includes detecting the signals emitted by the multivalent molecules that are bound to the complexed polymerases, wherein the complementary nucleotide units of the multivalent molecules are bound to the primers but incorporation of the complementary nucleotide units is inhibited. In some embodiments, the multivalent molecules are labeled with a detectable reporter moiety to permit detection. In some embodiments, the labeled multivalent molecules comprise a fluorophore attached to the core, linker and/or nucleotide unit of the multivalent molecules. In some embodiments, the detecting is carried out using an optical imaging system as described herein.

In some embodiments, the methods for sequencing further comprise step (d): identifying the nucleo-base of the complementary nucleotide units that are bound to the plurality of first complexed polymerases, thereby determining the sequence of the template molecule. In some embodiments, the multivalent molecules are labeled with a detectable reporter moiety that corresponds to the particular nucleotide units attached to the nucleotide arms to permit identification of the complementary nucleotide units (e.g., nucleotide base adenine, guanine, cytosine, thymine or uracil) that are bound to the plurality of first complexed polymerases.

In some embodiments, the methods for sequencing further comprise step (e): dissociating the plurality of multivalent-complexed polymerases and removing the plurality of first sequencing polymerases and their bound multivalent molecules, and retaining the plurality of nucleic acid duplexes.

In some embodiments, the second stage of the two-stage sequencing method comprises nucleotide incorporation. In some embodiments, the methods further comprises step (f): contacting the plurality of the retained nucleic acid duplexes of step (e) with a plurality of second sequencing polymerases, wherein the contacting is conducted under a condition suitable for binding the plurality of second sequencing polymerases to the plurality of the retained nucleic acid duplexes, thereby forming a plurality of second complexed polymerases each comprising a second sequencing polymerase bound to a nucleic acid duplex. In some embodiments, the second sequencing polymerase comprises a recombinant mutant sequencing polymerase.

In some embodiments, the plurality of first sequencing polymerases of step (a) have an amino acid sequence that is 100% identical to the amino acid sequence as the plurality of the second sequencing polymerases of step (f). In some embodiments, the plurality of first sequencing polymerases of step (a) have an amino acid sequence that differs from the amino acid sequence of the plurality of the second sequencing polymerases of step (f).

In some embodiments, the methods for sequencing further comprise step (g): contacting the plurality of second complexed polymerases with a plurality of nucleotides, wherein the contacting is conducted under conditions suitable for binding complementary nucleotides from the plurality of nucleotides to at least two of the second complexed polymerases thereby forming a plurality of nucleotide-complexed polymerases. In some embodiments, the contacting of step (g) is conducted under conditions that are suitable for promoting polymerase-catalyzed incorporation of the bound complementary nucleotides into the primers of the nucleotide-complexed polymerases thereby extending the sequencing primer by one nucleo-base. In some embodiments, the incorporating the nucleotide into the 3′ end of the sequencing primer in step (g) comprises a primer extension reaction. In some embodiments, the contacting of step (g) is conducted in the presence of at least one catalytic cation comprising magnesium and/or manganese. In some embodiments, the plurality of nucleotides comprise native nucleotides (e.g., non-analog nucleotides) or nucleotide analogs. In some embodiments, the plurality of nucleotides comprise a 2′ and/or 3′ chain terminating moiety which is removable or is not removable. In some embodiments, at least one of the nucleotides in the plurality is not labeled with a detectable reporter moiety. In some embodiments, the plurality of nucleotides are non-labeled. In some embodiments, the plurality of nucleotides comprises a plurality of nucleotides labeled with detectable reporter moiety. In some embodiments, the detectable reporter moiety comprises a fluorophore. In some embodiments, the fluorophore is attached to the nucleotide base. In some embodiments, the fluorophore is attached to the nucleotide base with a linker which is cleavable/removable from the base or is not removable from the base. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, when the plurality of nucleotides in step (g) are detectably labeled, the methods for sequencing further comprise step (h): detecting the complementary nucleotides which are incorporated into the primers of the nucleotide-complexed polymerases. In some embodiments, the plurality of nucleotides are labeled with a detectable reporter moiety to permit detection. In some embodiments, when the plurality of nucleotides in step (g) are non-labeled, the detecting of step (h) is omitted.

In some embodiments, when the plurality of nucleotides in step (g) are detectably labeled, the methods for sequencing further comprise step (i): identifying the bases of the complementary nucleotides which are incorporated into the primers of the nucleotide-complexed polymerases. In some embodiments, the identification of the incorporated complementary nucleotides in step (i) can be used to confirm the identity of the complementary nucleotides of the multivalent molecules that are bound to the plurality of first complexed polymerases in step (d). In some embodiments, the identifying of step (i) can be used to determine the sequence of the nucleic acid template molecules. In some embodiments, when the plurality of nucleotides in step (g) are non-labeled, the identifying of step (i) is omitted.

In some embodiments, the methods for sequencing further comprise step (j): removing the chain terminating moiety from the incorporated nucleotide when step (g) is conducted by contacting the plurality of second complexed polymerases with a plurality of nucleotides that comprise at least one nucleotide having a 2′ and/or 3′ chain terminating moiety.

In some embodiments, the methods for sequencing further comprise step (k): repeating steps (a)-(j) at least once. In some embodiments, the sequence of the nucleic acid template molecules can be determined by detecting and identifying the multivalent molecules that bind the sequencing polymerases but do not incorporate into the 3′ end of the primer at steps (c) and (d). In some embodiments, the sequence of the nucleic acid template molecule can be determined (or confirmed) by detecting and identifying the nucleotide that incorporates into the 3′ end of the primer at steps (h) and (i).

In some embodiments, the binding of the plurality of first complexed polymerases with the plurality of multivalent molecules forms at least one avidity complex, and the method comprises steps of: (a) binding a first nucleic acid primer, a first sequencing polymerase, and a first multivalent molecule to a first portion of a concatemer template molecule thereby forming a first binding complex, wherein a first nucleotide unit of the first multivalent molecule binds to the first sequencing polymerase; and (b) binding a second nucleic acid primer, a second sequencing polymerase, and the first multivalent molecule to a second portion of the same concatemer template molecule thereby forming a second binding complex, wherein a second nucleotide unit of the first multivalent molecule binds to the second sequencing polymerase, wherein the first and second binding complexes which include the same multivalent molecule form an avidity complex. In some embodiments, the first sequencing polymerase comprises any wild type or recombinant mutant polymerase described herein. In some embodiments, the second sequencing polymerase comprises any wild type or recombinant mutant polymerase described herein. The concatemer template molecule comprises tandem repeat sequences of a sequence of interest and at least one universal sequencing primer binding site. The first and second nucleic acid primers can bind to a sequencing primer binding site along the concatemer template molecule. Exemplary multivalent molecules are shown in FIGS. 17-21.

In some embodiments, the method comprises binding a plurality of first complexed polymerases with a plurality of multivalent molecules to form at least one avidity complex, and the method comprises steps of: (a) contacting a plurality of sequencing polymerases and a plurality of nucleic acid primers with different portions of a concatemer nucleic acid concatemer molecule to form at least first and second complexed polymerases on the same concatemer template molecule; (b) contacting a plurality of multivalent molecules with the at least first and second complexed polymerases on the same concatemer template molecule, under conditions suitable to bind a single multivalent molecule from the plurality to the first and second complexed polymerases, wherein at least a first nucleotide unit of the single multivalent molecule is bound to the first complexed polymerase which includes a first primer hybridized to a first portion of the concatemer template molecule thereby forming a first binding complex (e.g., first ternary complex), and wherein at least a second nucleotide unit of the single multivalent molecule is bound to the second complexed polymerase which includes a second primer hybridized to a second portion of the concatemer template molecule thereby forming a second binding complex (e.g., second ternary complex), wherein the contacting is conducted under conditions suitable to inhibit polymerase-catalyzed incorporation of the bound first and second nucleotide units in the first and second binding complexes, and wherein the first and second binding complexes which are bound to the same multivalent molecule form an avidity complex; (c) detecting the first and second binding complexes on the same concatemer template molecule, and (d) identifying the first nucleotide unit in the first binding complex thereby determining the sequence of the first portion of the concatemer template molecule, and identifying the second nucleotide unit in the second binding complex thereby determining the sequence of the second portion of the concatemer template molecule. In some embodiments, the plurality of sequencing polymerases comprise any wild type or recombinant mutant sequencing polymerase described herein. The concatemer template molecule comprises tandem repeat sequences of a sequence of interest and at least one universal sequencing primer binding site. The plurality of nucleic acid primers can bind to a sequencing primer binding site along the concatemer template molecule. Exemplary multivalent molecules are shown in FIGS. 17-21.

### Sequencing-by-Binding

The present disclosure provides methods for sequencing any of the immobilized template molecules described herein, wherein the sequencing methods comprise a sequencing-by-binding (SBB) procedure which employs non-labeled chain-terminating nucleotides. In some embodiments, the SBB method comprises the steps of: (a) sequentially contacting a primed template nucleic acid with at least two separate mixtures under ternary complex stabilizing conditions, wherein the at least two separate mixtures each include a polymerase and a nucleotide, whereby the sequentially contacting results in the primed template nucleic acid being contacted, under the ternary complex stabilizing conditions, with nucleotide cognates for first, second and third base type base types in the template; (b) examining the at least two separate mixtures to determine whether a ternary complex formed; and (c) identifying the next correct nucleotide for the primed template nucleic acid molecule, wherein the next correct nucleotide is identified as a cognate of the first, second or third base type if ternary complex is detected in step (b), and wherein the next correct nucleotide is imputed to be a nucleotide cognate of a fourth base type based on the absence of a ternary complex in step (b); (d) adding a next correct nucleotide to the primer of the primed template nucleic acid after step (b), thereby producing an extended primer; and (e) repeating steps (a) through (d) at least once on the primed template nucleic acid that comprises the extended primer. Exemplary sequencing-by-binding methods are described in U.S. Pat. Nos. 10,246,744 and 10,731,141, the contents of each of which are hereby incorporated by reference in their entireties.

### Methods for Sequencing Using Phosphate-Chain Labeled Nucleotides

The present disclosure provides methods for sequencing using immobilized sequencing polymerases which bind non-immobilized template molecules, wherein the sequencing reactions are conducted with phosphate-chain labeled nucleotides. In some embodiments, the sequencing methods comprise step (a): providing a support having a plurality of sequencing polymerases immobilized thereon. In some embodiments, the sequencing polymerases comprises processive DNA polymerases. In some embodiments, the sequencing polymerases comprises wild type or mutant DNA polymerases, including for example a Phi29 DNA polymerase. In some embodiments, the support comprises a plurality of separate compartments and a sequencing polymerase is immobilized to the bottom of a compartment. In some embodiments, the separate compartments comprise a silica bottom through which light can penetrate. In some embodiments, the separate compartments comprise a silica bottom configured with a nanophotonic confinement structure comprising a hole in a metal cladding film (e.g., aluminum cladding film). In some embodiments, the hole in the metal cladding has a small aperture, for example, approximately 70 nm. In some embodiments, the height of the nanophotonic confinement structure is approximately 100 nm. In some embodiments, the nanophotonic confinement structure comprises a zero-mode waveguide (ZMW). In some embodiments, the nanophotonic confinement structure contains a liquid.

In some embodiments, the sequencing method further comprises step (b): contacting the plurality of immobilized sequencing polymerases with a plurality of single stranded circular nucleic acid template molecules and a plurality of oligonucleotide sequencing primers, under conditions suitable for individual immobilized sequencing polymerase to bind to individual single stranded circular template molecules, and suitable for individual sequencing primers to hybridize to individual single stranded circular template molecules, thereby generating a plurality of polymerase/template/primer complexes. In some embodiments, the individual sequencing primers hybridize to a universal sequencing primer binding site on the single stranded circular template molecule.

In some embodiments, the sequencing method further comprises step (c): contacting the plurality of polymerase/template/primer complexes with a plurality of phosphate chain labeled nucleotides comprising an aromatic base, a five-carbon sugar (e.g., ribose or deoxyribose), and phosphate chain comprising 3-20 phosphate groups, wherein the terminal phosphate group is linked to a detectable reporter moiety (e.g., a fluorophore). The first, second and third phosphate groups can be referred to as alpha, beta and gamma phosphate groups. In some embodiments, a particular detectable reporter moiety which is attached to the terminal phosphate group corresponds to the nucleotide base (e.g., dATP, dGTP, dCTP, dTTP or dUTP) to permit detection and identification of the nucleo-base. In some embodiments, the plurality of polymerase/template/primer complexes are contacted with the plurality of phosphate chain labeled nucleotides under a condition suitable for polymerase-catalyzed nucleotide incorporation. In some embodiments, the sequencing polymerases are capable of binding a complementary phosphate chain labeled nucleotide and incorporating the complementary nucleotide opposite a nucleotide in a template molecule. In some embodiment, the polymerase-catalyzed nucleotide incorporation reaction cleaves between the alpha and beta phosphate groups thereby releasing a multi-phosphate chain linked to a fluorophore.

In some embodiments, the sequencing method further comprises step (d): detecting the fluorescent signals emitted by the phosphate chain labeled nucleotides that are bound by the sequencing polymerases, and incorporated into the terminal ends of the sequencing primers. In some embodiments, step (d) further comprises identifying the phosphate chain labeled nucleotides that are bound by the sequencing polymerases, and incorporated into the terminal ends of the sequencing primers.

In some embodiments, the sequencing method further comprises step (d): repeating steps (c)-(d) at least once. In some embodiments, sequencing methods that employ phosphate chain labeled nucleotides can be conducted according to the methods described in U.S. Pat. Nos. 7,170,050; 7,302,146; and/or 7,405,281, the contents of each of which are incorporated by reference herein.

### Sequencing Polymerases

The present disclosure provides methods for sequencing nucleic acid molecules, where any of the sequencing methods described herein employ at least one type of sequencing polymerase and a plurality of nucleotides, or employ at least one type of sequencing polymerase and a plurality of nucleotides and a plurality of multivalent molecules comprising nucleotide units. In some embodiments, the sequencing polymerase(s) is/are capable of incorporating a complementary nucleotide opposite a nucleotide in a template molecule. In some embodiments, the sequencing polymerase(s) is/are capable of binding a complementary nucleotide unit of a multivalent molecule opposite a nucleotide in a template molecule. In some embodiments, the plurality of sequencing polymerases comprises recombinant mutant polymerases.

Examples of suitable polymerases for use in sequencing with nucleotides and/or multivalent molecules include but are not limited to: Klenow DNA polymerase; Thermus aquaticus DNA polymerase I (Taq polymerase); KlenTaq polymerase; Candidatus altiarchaeales archaeon; Candidatus Hadarchaeum Yellowstonense; Hadesarchaea archaeon; Euryarchaeota archaeon; Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases; Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or O reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as 9 degrees N, VENT®, DEEP VENT®, THERMINATOR™, Pfu, KOD, Pfx, Tgo and RB69 polymerases.

### Nucleotides

The present disclosure provides methods for sequencing nucleic acid molecules, where any of the sequencing methods described herein employ at least one nucleotide. The nucleotides comprise a base, sugar and at least one phosphate group. In some embodiments, at least one nucleotide in the plurality comprises an aromatic base, a five carbon sugar (e.g., ribose or deoxyribose), and one or more phosphate groups (e.g., 1-10 phosphate groups). The plurality of nucleotides can comprise at least one type of nucleotide selected from a group consisting of dATP, dGTP, dCTP, dTTP and dUTP. The plurality of nucleotides can comprise at a mixture of any combination of two or more types of nucleotides selected from a group consisting of dATP, dGTP, dCTP, dTTP and/or dUTP. In some embodiments, at least one nucleotide in the plurality is not a nucleotide analog. In some embodiments, at least one nucleotide in the plurality comprises a nucleotide analog.

In some embodiments, in any of the methods for sequencing nucleic acid molecules described herein, at least one nucleotide in the plurality of nucleotides comprise a chain of one, two or three phosphorus atoms wherein the chain is typically attached to the 5′ carbon of the sugar moiety via an ester or phosphoramide linkage. In some embodiments, at least one nucleotide in the plurality is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

In some embodiments, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to 3′ sugar position wherein the sugar comprises a ribose or deoxyribose sugar moiety. In some embodiments, the chain terminating moiety is removable/cleavable from 3′ sugar position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, silyl or acetal group. In some embodiments, the chain terminating moiety is cleavable/removable from the nucleotide, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, and/or disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride. In some embodiments, the chain terminating moiety may be cleavable/removable with nitrous acid. In some embodiments, a chain terminating moiety may be cleavable/removable using a solution comprising nitrite, such as, for example, a combination of nitrite with an acid such as acetic acid, sulfuric acid, or nitric acid. In some further embodiments, said solution may comprise an organic acid.

In some embodiments, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP). In some embodiments, the chain terminating moiety comprising one or more of a 3′-O-amino group, a 3′-O-aminomethyl group, a 3′-O-methylamino group, or derivatives thereof may be cleaved with nitrous acid, through a mechanism utilizing nitrous acid, or using a solution comprising nitrous acid. In some embodiments, the chain terminating moiety comprising one or more of a 3′-O-amino group, a 3′-O-aminomethyl group, a 3′-O-methylamino group, or derivatives thereof may be cleaved using a solution comprising nitrite. In some embodiments, for example, nitrite may be combined with or contacted with an acid such as acetic acid, sulfuric acid, or nitric acid. In some further embodiments, for example, nitrite may be combined with or contacted with an organic acid such as, for example, formic acid, acetic acid, propionic acid, butyric acid, isobutyric acid, or the like. In some embodiments, the chain terminating moiety comprises a 3′-acetal moiety which can be cleaved with a palladium deblocking reagent (e.g., Pd(0)).

In some embodiments, in any of the methods for sequencing nucleic acid molecules described herein, the nucleotide comprises a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, 3-O-benzyl, and 3′-O-benzyl, 3-acetal moiety or derivatives thereof.

In some embodiments, in any of the methods for sequencing nucleic acid molecules described herein, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, in any of the methods for sequencing nucleic acid molecules described herein, the cleavable linker on the nucleotide base comprises a cleavable moiety comprising an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the cleavable linker on the base is cleavable/removable from the base by reacting the cleavable moiety with a chemical agent, pH change, light or heat. In some embodiments, the cleavable moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the cleavable moieties aryl and benzyl are cleavable with H2 Pd/C. In some embodiments, the cleavable moieties amine, amide, keto, isocyanate, phosphate, thio, and/or disulfide are cleavable with phosphine or with a thiol group including beta-mercaptoethanol or dithiothritol (DTT). In some embodiments, the cleavable moiety carbonate is cleavable with potassium carbonate (K₂CO₃) in MeOH, with triethylamine in pyridine, or with Zn in acetic acid (AcOH). In some embodiments, the cleavable moieties urea and silyl are cleavable with tetrabutylammonium fluoride, pyridine-HF, with ammonium fluoride, or with triethylamine trihydrofluoride.

In some embodiments, in any of the methods for sequencing nucleic acid molecules described herein, the cleavable linker on the nucleotide base comprises cleavable moiety including an azide, azido or azidomethyl group. In some embodiments, the cleavable moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the methods for sequencing nucleic acid molecules described herein, the chain terminating moiety (e.g., at the sugar 2′ and/or sugar 3′ position) and the cleavable linker on the nucleotide base have the same or different cleavable moieties. In some embodiments, the chain terminating moiety (e.g., at the sugar 2′ and/or sugar 3′ position) and the detectable reporter moiety linked to the base are chemically cleavable/removable with the same chemical agent. In some embodiments, the chain terminating moiety (e.g., at the sugar 2′ and/or sugar 3′ position) and the detectable reporter moiety linked to the base are chemically cleavable/removable with different chemical agents.

### Reporter Moieties

The disclosure provides detectable reporter moieties, for use with the labeled nucleotides and multivalent molecules of the disclosure.

The term “reporter moiety”, “reporter moieties” or “detectable reporter moieties” and related terms refers to a compound that generates, or causes to generate, a detectable signal. A reporter moiety is sometimes called a “label”. Any suitable reporter moiety may be used, including luminescent, photoluminescent, electroluminescent, bioluminescent, chemiluminescent, fluorescent, phosphorescent, chromophore, radioisotope, electrochemical, mass spectrometry, Raman, hapten, affinity tag, atom, or an enzyme. A reporter moiety generates a detectable signal resulting from a chemical or physical change (e.g., heat, light, electrical, pH, salt concentration, enzymatic activity, or proximity events). A proximity event includes two reporter moieties approaching each other, or associating with each other, or binding each other. It is well known to one skilled in the art to select reporter moieties so that each absorbs excitation radiation and/or emits fluorescence at a wavelength distinguishable from the other reporter moieties to permit monitoring the presence of different reporter moieties in the same reaction or in different reactions. Two or more different reporter moieties can be selected having spectrally distinct emission profiles, or having minimal overlapping spectral emission profiles. Reporter moieties can be linked (e.g., operably linked) to nucleotides, nucleosides, nucleic acids, enzymes (e.g., polymerases or reverse transcriptases), or support (e.g., surfaces).

A reporter moiety (or label) comprises a fluorescent label or a fluorophore. Exemplary fluorescent moieties which may serve as fluorescent labels or fluorophores include, but are not limited to fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY™ FL C3-SE, BODIPY™ 530/550 C3, BODIPY™ 530/550 C3-SE, BODIPY™ 530/550 C3 hydrazide, BODIPY™ 493/503 C3 hydrazide, BODIPY™ FL C3 hydrazide, BODIPY™ FL IA, BODIPY™ 530/551 IA, Br-BODIPY™ 493/503, Cascade Blue™ and derivatives such as Cascade Blue™ acetyl azide, Cascade Blue™ cadaverine, Cascade Blue™ ethylenediamine, Cascade Blue™ hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor® dyes, DyLight™ dyes, Atto dyes, LightCycler® Red dyes, CAL Flour dyes, JOE and derivatives thereof, Oregon Green dyes, WellRED dyes, IRD dyes, phycoerythrin and phycobilin dyes, Malachite green, stilbene, DEG dyes, NR dyes, near-infrared dyes and others known in the art such as those described in Haugland, Molecular Probes Handbook, (Eugene, Oreg.) 6th Edition; Lakowicz, Principles of Fluorescence Spectroscopy, 2nd Ed., Plenum Press New York (1999), or Hermanson, Bioconjugate Techniques, 2nd Edition, or derivatives thereof, or any combination thereof. Cyanine dyes may exist in either sulfonated or non-sulfonated forms, and consist of two indolenin, benzo-indolium, pyridium, thiozolium, and/or quinolinium groups separated by a polymethine bridge between two nitrogen atoms. Commercially available cyanine fluorophores include, for example, Cy3, (which may comprise 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-1,3-dihydro-2H-indol-2-ylidene}prop-1-en-1-yl)-3,3-dimethyl-3H-indolium or 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-5-sulfo-1,3-dihydro-2H-indol-2-ylidene}prop-1-en-1-yl)-3,3-dimethyl-3H-indolium-5-sulfonate), Cy5 (which may comprise 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-indolin-2-ylidene) penta-1,3-dien-1-yl)-3,3-dimethyl-3H-indol-1-ium or 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-sulfoindolin-2-ylidene) penta-1,3-dien-1-yl)-3,3-dimethyl-3H-indol-1-ium-5-sulfonate), and Cy7 (which may comprise 1-(5-carboxypentyl)-2-[(1E,3E,5E,7Z)-7-(1-ethyl-1,3-dihydro-2H-indol-2-ylidene) hepta-1,3,5-trien-1-yl]-3H-indolium or 1-(5-carboxypentyl)-2-[(1E,3E,5E,7Z)-7-(1-ethyl-5-sulfo-1,3-dihydro-2H-indol-2-ylidene) hepta-1,3,5-trien-1-yl]-3H-indolium-5-sulfonate), where “Cy” stands for ‘cyanine’, and the first digit identifies the number of carbon atoms between two indolenine groups. Cy2 which is an oxazole derivative rather than indolenin, and the benzo-derivatized Cy3.5, Cy5.5 and Cy7.5 are exceptions to this rule.

In some embodiments, the reporter moiety can be a FRET pair, such that multiple classifications can be performed under a single excitation and imaging step. As used herein, FRET may comprise excitation exchange (Forster) transfers, or electron-exchange (Dexter) transfers.

### Multivalent Molecules

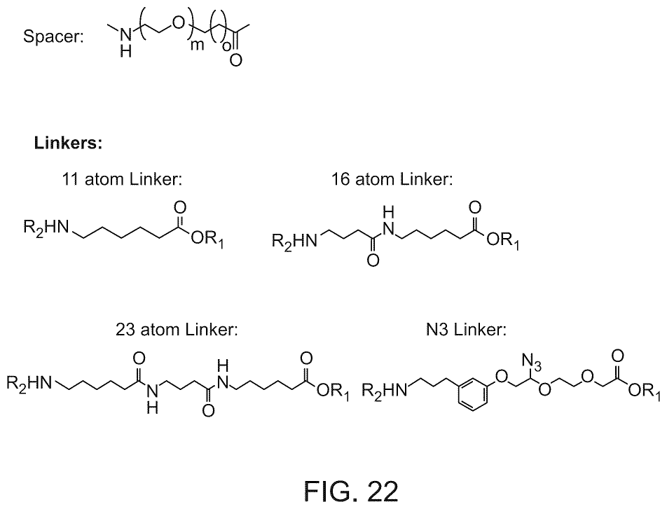

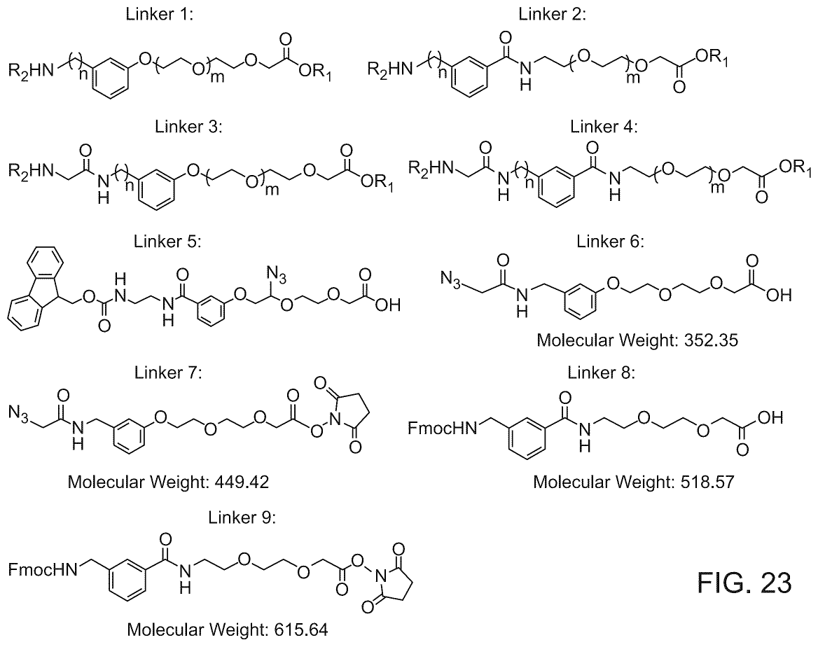

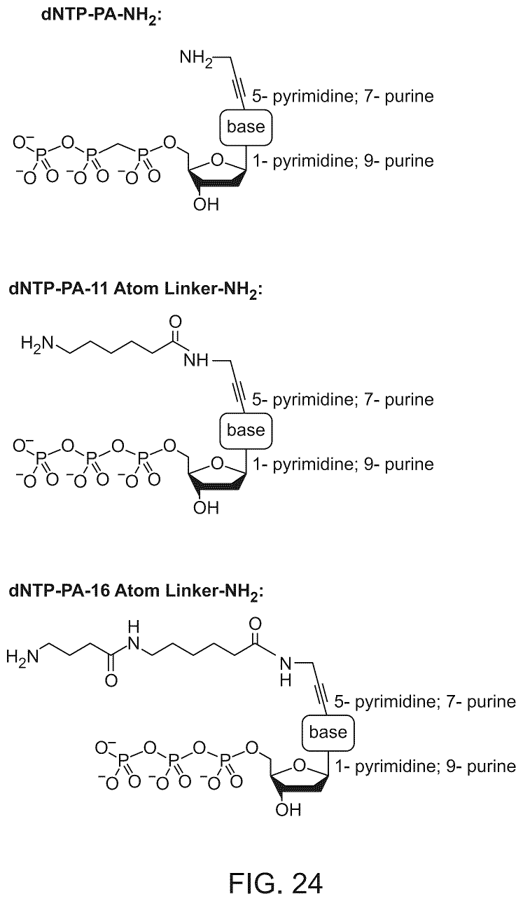

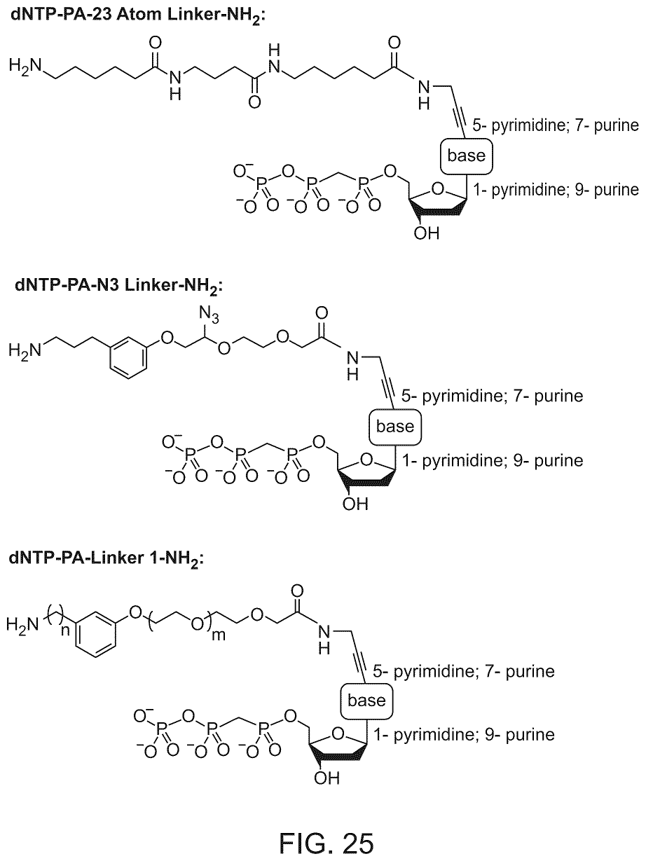

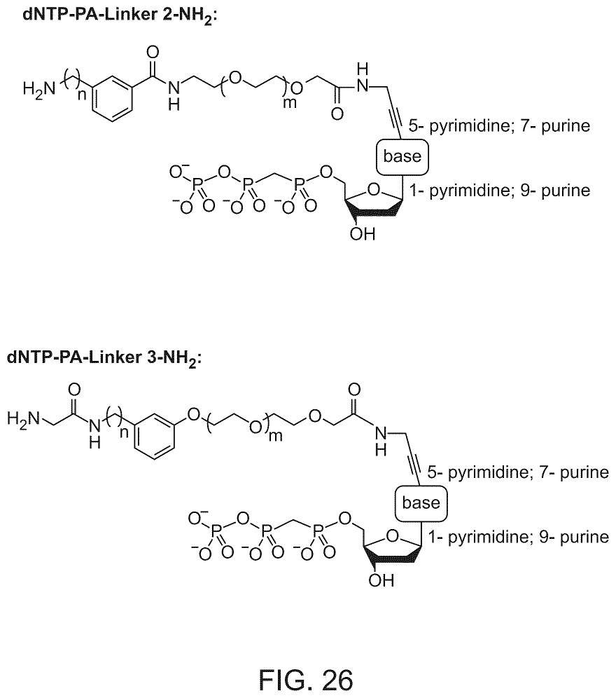

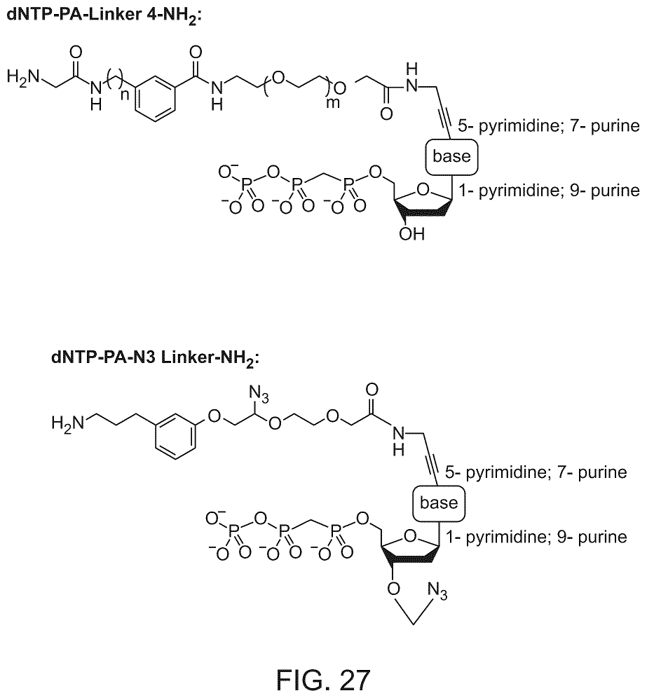

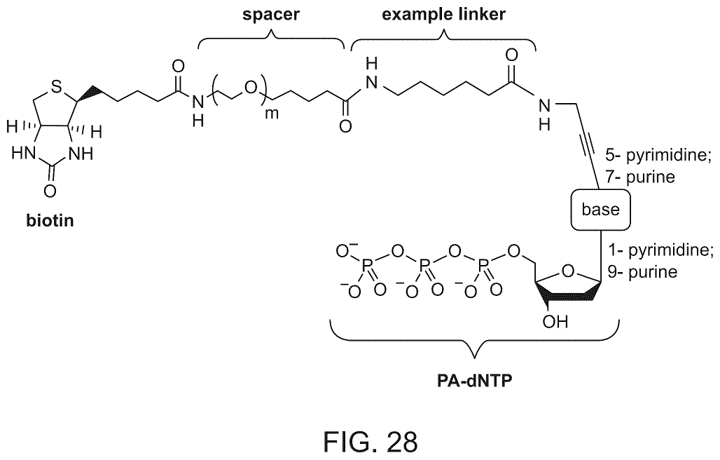

The present disclosure provides methods for sequencing nucleic acid molecules, where any of the sequencing methods described herein employ at least one multivalent molecule. In some embodiments, the multivalent molecule comprises a plurality of nucleotide arms attached to a core and having any configuration including a starburst, helter skelter, or bottle brush configuration (e.g., FIG. 17). In some embodiments, the multivalent molecule comprises: (1) a core; and (2) a plurality of nucleotide arms which comprise (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, and wherein the linker is attached to the nucleotide unit. In some embodiments, the nucleotide unit comprises a base, sugar and at least one phosphate group, and the linker is attached to the nucleotide unit through the base. In some embodiments, the linker comprises an aliphatic chain or an oligo ethylene glycol chain where both linker chains having 2-6 subunits. In some embodiments, the linker also includes an aromatic moiety. An exemplary nucleotide arm is shown in FIG. 21. Exemplary multivalent molecules are shown in FIGS. 17-20. An exemplary spacer is shown in FIG. 22 (top) and exemplary linkers are shown in FIG. 22 (bottom) and FIG. 15. Exemplary nucleotides attached to a linker are shown in FIGS. 24-27. An exemplary biotinylated nucleotide arm is shown in FIG. 28.

In some embodiments, a multivalent molecule comprises a core attached to multiple nucleotide arms, and wherein the multiple nucleotide arms have the same type of nucleotide unit which is selected from a group consisting of dATP, dGTP, dCTP, dTTP and dUTP.

In some embodiments, a multivalent molecule comprises a core attached to multiple nucleotide arms, wherein each arm includes a nucleotide unit. The nucleotide unit comprises an aromatic base, a five-carbon sugar (e.g., ribose or deoxyribose), and one or more phosphate groups (e.g., 1-10 phosphate groups). The plurality of multivalent molecules can comprise one type of multivalent molecule having one type of nucleotide unit selected from a group consisting of dATP, dGTP, dCTP, dTTP and dUTP. The plurality of multivalent molecules can comprise at a mixture of any combination of two or more types of multivalent molecules, where individual multivalent molecules in the mixture comprise nucleotide units selected from a group consisting of dATP, dGTP, dCTP, dTTP and/or dUTP, such that individual multivalent molecules comprise a single type of nucleotide unit (e.g., dATP on all arms of the individual multivalent molecule).

In some embodiments, the nucleotide unit comprises a chain of one, two or three phosphorus atoms wherein the chain is typically attached to the 5′ carbon of the sugar moiety via an ester or phosphoramide linkage. In some embodiments, at least one nucleotide unit is a nucleotide analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

In some embodiments, the multivalent molecule comprises a core attached to multiple nucleotide arms, wherein individual nucleotide arms comprise a nucleotide unit which is a nucleotide analog having a chain terminating moiety (e.g., blocking moiety) at the sugar 2′ position, at the sugar 3′ position, or at the sugar 2′ and 3′ position. In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to 3′ sugar position wherein the sugar comprises a ribose or deoxyribose sugar moiety. In some embodiments, the chain terminating moiety is removable/cleavable from 3′ sugar position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable/removable from the nucleotide unit, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, and/or disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, the nucleotide unit comprising a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, the multivalent molecule comprises a core attached to multiple nucleotide arms, wherein the nucleotide arms comprise a spacer, linker and nucleotide unit, and wherein the core, linker and/or nucleotide unit is labeled with a detectable reporter moiety. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, at least one nucleotide arm of a multivalent molecule has a nucleotide unit that is attached to a detectable reporter moiety. In some embodiments, the detectable reporter moiety is attached to the nucleotide base. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, the core of a multivalent molecule comprises an avidin-like or streptavidin-like moiety and the core attachment moiety comprises biotin. In some embodiments, the core comprises an streptavidin-type or avidin-type moiety which includes an avidin protein, as well as any derivatives, analogs and other non-native forms of avidin that can bind to at least one biotin moiety. Other forms of avidin moieties include native and recombinant avidin and streptavidin as well as derivatized molecules, e.g., non-glycosylated avidin and truncated streptavidins. For example, avidin moiety includes de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially-available products EXTRAVIDIN®, CAPTAVIDIN™, NEUTRAVIDIN™ and NEUTRALITE AVIDIN.

In any of the embodiments of methods for sequencing nucleic acid molecules described herein that include forming a binding complex, the binding complex can comprise (i) a polymerase, a nucleic acid template molecule duplexed with a primer, and a nucleotide, or the binding complex comprises (ii) a polymerase, a nucleic acid template molecule duplexed with a primer, and a nucleotide unit of a multivalent molecule. In some embodiments, the binding complex has a persistence time of greater than about 0.1, 0.2, 0.3, 0.4, 0.5, 0.6, 0.7, 0.8, 0.9 or 1 second. In some embodiments, the binding complex has a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds, and/or wherein the method is or may be carried out at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing. In some embodiments, the binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water. In some embodiments, the present disclosure provides said method wherein the binding complex is deposited on, attached to, or hybridized to, a surface showing a contrast to noise ratio in the detecting step of greater than 20. In some embodiments, the present disclosure provides said method wherein the contacting is performed under a condition that stabilizes the binding complex when the nucleotide or nucleotide unit is complementary to a next base of the template nucleic acid, and destabilizes the binding complex when the nucleotide or nucleotide unit is not complementary to the next base of the template nucleic acid.

### Compaction Oligonucleotides

The disclosure provides compaction oligos for use in the sequencing methods described herein.

A compaction oligonucleotide comprises a single-stranded linear oligonucleotide having a 5′ region that can hybridize to a first portion of a concatemer molecule and the compaction oligonucleotide having a 3′ region that can hybridize to a second portion of the concatemer molecule (e.g., the same concatemer molecule). In some embodiments, hybridization of the compaction oligonucleotides to individual concatemer molecules causes the concatemer molecule to collapse or fold into a DNA nanoball which is more compact in shape and size compared to a non-collapsed DNA molecule. A spot image of a DNA nanoball can be represented as a Gaussian spot and the size can be measured as a full width half maximum (FWHM). A smaller spot size as indicated by a smaller FWHM typically correlates with an improved image of the spot. In some embodiments, the FWHM of a DNA nanoball spot can be about 10 um or smaller. The DNA nanoball can be a compact nucleic acid structure having a full width half maximum (FWHM) that is smaller compared to a concatemer that is not collapsed/folded into a DNA nanoball.

In some embodiments, compaction oligonucleotides comprise a single stranded oligonucleotides comprising DNA, RNA, or a combination of DNA and RNA. The compaction oligonucleotides can be any length, including 20-150 nucleotides, or 30-100 nucleotides, or 40-80 nucleotides in length.

In some embodiments, the compaction oligonucleotides comprise a 5′ region and a 3′ region, and optionally an intervening region between 5′ and 3′ regions. The intervening region can be any length, for example about 2-20 nucleotides in length. The intervening region comprises a homopolymer having consecutive identical bases (e.g., AAA, GGG, CCC, TTT or UUU). The intervening region comprises a non-homopolymer sequence.

The 5′ region of the compaction oligonucleotides can be wholly complementary or partially complementary along its length to a first portion of a concatemer molecule. The 3′ region of the compaction oligonucleotides can be wholly complementary or partially complementary along its length to a second portion of a concatemer molecule. The 5′ region of the compaction oligonucleotides can hybridize to a first universal sequence portion of a concatemer molecule. The 3′ region of the compaction oligonucleotides can hybridize to a second universal sequence portion of a concatemer molecule. The 5′ and 3′ regions of the compaction oligonucleotide can hybridize to the concatemer to pull together distal portions of the concatemer causing compaction of the concatemer to form a DNA nanoball.

The 5′ region of the compaction oligonucleotide can have the same sequence as 3′ region. The 5′ region of the compaction oligonucleotide can have a sequence that is different from 3′ region. The 3′ region of the compaction oligonucleotide can have a sequence that is a reverse sequence of 5′ region.

### Nanopore Sequencing

In some embodiments, sequence data may be derived through nanopore sequencing, which comprises sequencing of a nucleic acid by translocating said nucleic acid across a membrane, such as through a pore, wherein sequence reads or base calls are made by measuring one or more signals during the translocation event, such as impedance, current, voltage, or capacitance. In some embodiments, the identity of a nucleotide may be determined by distinctive electrical signatures, such as the timing, duration, extent, or line shape of a current block, impedance change, voltage change, or capacitance change. Sequencing of nucleic acids by translocation across a membrane and/or through a pore does not foreclose alternative detection methods, such as optical, chemical, biochemical, fluorescent, luminescent, magnetic, electromagnetic, acoustic, or electroacoustic detection.

### Contrast to Noise Ratio

In some embodiments, the performance of nucleic acid hybridization and/or amplification reactions using the disclosed reaction formulations and low-binding supports may be assessed using fluorescence imaging techniques, wherein the contrast-to-noise ratio (CNR) of the images provides a key metric in assessing amplification specificity and non-specific binding on the support. CNR is described in US 2020/0149095, which is incorporated by reference herein, and is commonly defined as: CNR=(Signal-Background)/Noise. The background term is commonly taken to be the signal measured for the interstitial regions surrounding a particular feature (diffraction limited spot, DLS) in a specified region of interest (ROI). While signal-to-noise ratio (SNR) is often considered to be a benchmark of overall signal quality, it can be shown that improved CNR can provide a significant advantage over SNR as a benchmark for signal quality in applications that require rapid image capture (e.g., sequencing applications for which cycle times must be minimized), as shown in the example below. At high CNR the imaging time required to reach accurate discrimination (and thus accurate base-calling in the case of sequencing applications) can be drastically reduced even with moderate improvements in CNR. Improved CNR in imaging data on the imaging integration time provides a method for more accurately detecting features such as clonally-amplified nucleic acid colonies on the support surface.

In most ensemble-based sequencing approaches, the background term is typically measured as the signal associated with ‘interstitial’ regions. In addition to “interstitial” background (Bᵢₙₜₑᵣ), “intrastitial” background (Bᵢₙₜᵣₐ) exists within the region occupied by an amplified DNA colony. The combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. The Bᵢₙₜₑᵣ background signal arises from a variety of sources; a few examples include auto-fluorescence from consumable flow cells, non-specific adsorption of detection molecules that yield spurious fluorescence signals that may obscure the signal from the ROI, the presence of non-specific DNA amplification products (e.g., those arising from primer dimers). In typical next generation sequencing (NGS) applications, this background signal in the current field-of-view (FOV) is averaged over time and subtracted. The signal arising from individual DNA colonies (i.e., (Signal)-B(interstial) in the FOV) yields a discernable feature that can be classified. In some embodiments, the intrastitial background (B(intrastitial)) can contribute a confounding fluorescence signal that is not specific to the target of interest, but is present in the same ROI thus making it far more difficult to average and subtract.

Nucleic acid amplification on the low-binding coated supports described herein may decrease the B (interstitial) background signal by reducing non-specific binding, may lead to improvements in specific nucleic acid amplification, and may lead to a decrease in non-specific amplification that can impact the background signal arising from both the interstitial and intrastitial regions. In some embodiments, the disclosed low-binding coated supports, optionally used in combination with the disclosed hybridization and/or amplification reaction formulations, may lead to improvements in CNR by a factor of 2, 5, 10, 100, 250, 500 or 1000-fold over those achieved using conventional supports and hybridization, amplification, and/or sequencing protocols. Although described here in the context of using fluorescence imaging as the read-out or detection mode, the same principles apply to the use of the disclosed low-binding coated supports and nucleic acid hybridization and amplification formulations for other detection modes as well, including both optical and non-optical detection modes.

### ENUMERATED EMBODIMENTS

The disclosure can be further understood with reference to the following enumerated embodiments:

1. A solid-state optical test target comprising:
a) a first substrate comprising a transparent medium, and having top surface, bottom surface and one or more side surfaces, the top and bottom surfaces being flat, and the first substrate having a refractive index of [n-top substrate(1)]; and
b) a second substrate having top surface, bottom surface and side surfaces, the top surface being flat, wherein
   (i) at least a portion of the top surface of the second substrate comprises an opaque coating that forms a micropattern, the micropattern configured to include opaque portions and transparent portions,
   (ii) the first substrate is positioned on top of the second substrate, and the first substrate is positioned in direct contact with the micropattern on the second substrate,
   (iii) the solid-state optical test target lacks a flow cell and lacks a liquid,
   (iv) the thickness of the first substrate simulates the presence of a first hypothetical flow cell located between the first and second substrates, wherein the first hypothetical flow cell includes a first channel having a top surface and bottom surface, the first channel containing a first fluid, wherein the first channel has a first thickness of [T-channel(1)] and the first fluid has a refractive index of [n-fluid(1)], and
   (v) the thickness of the first substrate is configured to permit imaging of the bottom surface of the first channel of the first hypothetical flow cell.
      2. The solid-state optical test target of embodiment 1, wherein the top surface, bottom surface and one or more side surfaces have an even thickness.
      3. The solid-state optical test target of embodiment 1 or 2, wherein the first substrate comprises transparent glass.
      4. The solid-state optical test target of any one of embodiments 1-3, wherein the second substrate comprises transparent glass.
      5. The solid-state optical test target of any one of embodiments 1-4, wherein the top, side and/or bottom surfaces of the second substrate are transparent to permit light transmission through the top, side and/or bottom surfaces.
      6. The solid-state optical test target of any one of embodiments 1-5, wherein the thickness of the opaque coating that forms the micropattern on the top surface of the second substrate is about 100 nm.
      7. The solid-state optical test target of any one of embodiments 1-6, wherein the opaque coating comprises chromium or aluminum.
      8. The solid-state optical test target of any one of embodiments 1-7, wherein the transparent portions of the micropattern comprise regions of the top surface of the second substrate without the opaque coating.
      9. The solid-state optical test target of any one of embodiments 1-8, wherein the transparent portions of the micropattern comprise repeating shapes arranged in an array.
      10. The solid-state optical test target of any one of embodiments 1-9, wherein the transparent portions of the micropattern form a plurality of one type of shape, or a mixture of different types of shapes, wherein the one type of shape and the mixture of different types of shapes are selected from a group consisting of circles, squares and triangles.
      11. The solid-state optical test target of any one of embodiments 1-10, wherein the transparent portions of the micropattern forms the shape of at least one line.
      12. The solid-state optical test target of any one of embodiments 1-11, wherein the transparent portions of the micropattern form at least one alphanumeric character.
      13. The solid-state optical test target of any one of embodiments 1-12, wherein the transparent portions of the micropattern form a plurality of pinholes, wherein the plurality of pinholes fill a full field of view when the solid-state optical test target is positioned in an optical imaging system which comprises at least one objective lens, at least one tube lens, and a camera.
      14. The solid-state optical test target of any one of embodiments 1-13, wherein the transparent portions of the micropattern form a non-repeating rotationally symmetrical shape including concentric circles.
      15. The solid-state optical test target of embodiment 14, wherein the symmetrical shape comprises a bullseye or a plus sign (+).
      16. The solid-state optical test target of any one of embodiments 9-15, wherein the dimension of the transparent portions of the micropattern is about 1 micron.
      17. The solid-state optical test target of any one of embodiments 1-16, wherein the opaque portions of the micropattern comprising repeating shapes arranged in an array.
      18. The solid-state optical test target of any one of embodiments 1-17, wherein the opaque portions of the micropattern form a plurality of one type of shape, or a mixture of different types of shapes, where the one type of shape and the mixture of different types of shapes are selected from a group consisting of circles, squares and triangles.
      19. The solid-state optical test target of any one of embodiments 1-15, wherein the opaque portions of the micropattern form the shape of at least one line.
      20. The solid-state optical test target of any one of embodiments 1-19, wherein the opaque portions of the micropattern form at least one alphanumeric character.
      21. The solid-state optical test target of any one of embodiments 1-20, wherein the opaque portions of the micropattern form a plurality of pinholes, wherein the plurality of pinholes fill a full field of view when the solid-state optical test target is positioned in an optical imaging system which comprises at least one objective lens, at least one tube lens, and a camera.
      22. The solid-state optical test target of any one of embodiments 1-21, wherein the opaque portions of the micropattern form a non-repeating rotationally symmetrical shape including concentric circles.
      23. The solid-state optical test target of embodiment 22, wherein the symmetrical shape comprises a bullseye or plus sign (+).
      24. The solid-state optical test target of any one of embodiments 17-23, wherein the dimension of the opaque portions of the micropattern is about 1 micron.
      25. The solid-state optical test target of any one of embodiments 1-24, wherein the height/thickness of the first substrate [T-top substrate(1)] is related to the refractive index of the first substrate [n-top substrate(1)], the first height of the first channel [T-channel(1)] and the refractive index of the first fluid [n-fluid(1)], in an equation

[T-top substrate(1)]=([(T-channel(1)]*([(n-fluid(1)]/[n-top substrate(1)])) (Equation 2).

26. The solid-state optical test target of any one of embodiments 1-25, which is positioned in an optical imaging system which comprises at least one objective lens, at least one tube lens, and a camera.
27. The solid-state optical test target of embodiment 26, wherein the optical imaging system further comprises at least on light source and at least one filter.
28. The solid-state optical test target of embodiment 27, wherein the at least on light source comprises a laser or LED excitation light.
29. The solid-state optical test target of embodiment 27 or 28, wherein the at least one light source is positioned to excite a fluorophore in a sample.
30. The solid-state optical test target of any one of embodiments 1-29, wherein the first substrate is removable from the second substrate.
31. The solid-state optical test target of any one of embodiments 1-30, wherein the first substrate is replaced with a third substrate which comprises a transparent medium and having a top surface, a bottom surface and one or more side surfaces, and the third substrate having a refractive index of [n-top substrate(3)], wherein
(i) the third substrate is positioned on top of the second substrate, and the third substrate is positioned in direct contact with the micropattern on the second substrate,
(ii) the solid-state optical test target lacks a flow cell and lacks a liquid,
(iii) the height (thickness) of the third substrate is configured to simulate the presence of a second hypothetical flow cell located between the third and second substrates, wherein the second hypothetical flow cell includes a second channel having a top surface and a bottom surface, and the second channel containing a second fluid, wherein the second channel has a second thickness of [T-channel(2)] and the second fluid has a refractive index of [n-fluid(2)], and
(iv) the thickness of the third substrate is configured to permit imaging of the bottom surface of the second channel of the second hypothetical flow cell.
   32. The solid-state optical test target of any one of embodiments 1-31, wherein the top surface, bottom surface and one or more side surfaces have an even thickness.
   33. The solid-state optical test target of any one of embodiments 1-32, wherein the third substrate comprises transparent glass.
   34. The solid-state optical test target of any one of embodiments 1-32, wherein the height/thickness of the third substrate [T-top substrate(3)] is related to the refractive index of the third substrate [n-top substrate(3)], the second height of the second channel [T-channel(2)] and the refractive index of the second fluid [n-fluid(2)], in an equation

[T-top substrate(3)]=([(T-channel(2)]*([(n-fluid(2)]/[n-top substrate(3)])) (Equation 3).

35. The solid-state optical test target of embodiments 31 or 34, wherein the refractive index of the first substrate [n-top substrate(1)] is the same or different from the refractive index of the third substrate [n-top substrate(3)].
36. The solid-state optical test target of embodiments 31 or 34, wherein the height of the first hypothetical channel [T-channel(1)] is the same or different from the height of the second hypothetical channel [T-channel(2)].
37. The solid-state optical test target of embodiments 31 or 34, wherein the refractive index of the first fluid [n-fluid(1)] is the same or different from the refractive index of the second fluid [n-fluid(2)].
38. The solid-state optical test target of any one of embodiments 1-37, which is positioned in an optical imaging system which comprises at least one objective lens, at least one tube lens, and a camera.
39. The solid-state optical test target of any one of embodiments 1-38, wherein the optical imaging system further comprises at least on light source and at least one filter.
40. The solid-state optical test target of any one of embodiments 1-39, wherein the bottom surface of the second substrate comprises a reflecting coating, or wherein the bottom surface of the second substrate comprises a rough scatter surface.
41. The solid-state optical test target of embodiment 31, wherein the second substrate comprises a first fluorescent microscope slide that provides a continuous fluorescent field and comprises a material that has first fluorescence spectrum.
42. The solid-state optical test target of any one of embodiments 1-34, wherein first fluorescence spectrum comprises a green band emission.
43. The solid-state optical test target of any one of embodiments 1-42, comprising a second fluorescent microscope slide located under the first fluorescent microscope slide, wherein the second fluorescent microscope slide provides a continuous fluorescent field and has a second fluorescence spectrum that differs or does not substantially overlap with the first fluorescence spectrum of the first fluorescent microscope slide.
44. The solid-state optical test target of any one of embodiments 1-43, wherein the second fluorescent microscope slide has a fluorescence spectrum that produces a red band emission.
45. The solid-state optical test target of any one of embodiments 31-44, wherein the second substrate comprises a planar-shaped LED light.
46. An adaptive solid-state optical test target comprising:
a) a first substrate comprising a transparent medium and a top surface, a bottom surface and one or more side surfaces, the first substrate having at least two regions comprising different thicknesses, wherein the first region and has a first thickness and the second region and has a second thickness, and the first substrate comprises a refractive index of [n-top substrate(1)]; and
b) a second substrate comprising a top surface, a bottom surface and one or more side surfaces, the top surface being flat, wherein
   (i) at least a portion of the top surface of the second substrate comprises an opaque coating that forms a micropattern, wherein the micropattern is configured to include opaque portions and transparent portions,
   (ii) the first substrate is positioned on top of the second substrate, and the first substrate is positioned in direct contact with the micropattern on the second substrate,
   (iii) the solid-state optical test target lacks a flow cell and lacks a liquid,
   (iv) the thickness of the first region of the first substrate is configured to simulate the presence of a first hypothetical flow cell located between the first and second substrates, wherein the first hypothetical flow cell includes a first channel having a top surface and a bottom surface, and the first channel comprises a first fluid, wherein the first channel has a first thickness of [T-channel(1)] and the first fluid has a refractive index of [n-fluid(1)],
   (v) the thickness of the first region of the first substrate is configured to permit imaging of the bottom surface of the first channel of the first hypothetical cell, and
   (vi) the thickness of the second region of the first substrate is configured to permit imaging of the top surface of the first channel of the first hypothetical cell.
      47. The adaptive solid-state optical test target of embodiment 46, wherein the bottom surface is flat.
      48. The adaptive solid-state optical test target of embodiment 46 or 47, wherein the first and second regions are flat.
      49. The adaptive solid-state optical test target of any one of embodiments 46-48, wherein the transparent medium comprises transparent glass.
      50. The adaptive solid-state optical test target of any one of embodiments 46-49, wherein the second substrate comprises transparent glass.
      51. The adaptive solid-state optical test target of any one of embodiments 46-50, wherein the top surface, one or more side surfaces and/or bottom surface of the second substrate are transparent to permit light transmission through the top surface, one or more side surfaces and/or bottom surface.
      52. The adaptive solid-state optical test target of any one of embodiments 46-51, wherein the thickness of the opaque coating that forms the micropattern on the top surface of the second substrate is about 100 nm.
      53. The adaptive solid-state optical test target of any one of embodiments 46-52, wherein the opaque coating comprises chromium or aluminum.
      54. The adaptive solid-state optical test target of any one of embodiments 46-53, wherein the transparent portions of the micropattern comprise repeating shapes arranged in an array.
      55. The adaptive solid-state optical test target of any one of embodiments 46-54, wherein the transparent portions of the micropattern form a plurality of one type of shape, or a mixture of different types of shapes, where the one type of shape and the mixture of different types of shapes are selected from a group consisting of circles, squares and triangles.
      56. The adaptive solid-state optical test target of any one of embodiments 46-55, wherein the transparent portions of the micropattern forms the shape of at least one line.
      57. The adaptive solid-state optical test target of any one of embodiments 46-56, wherein the transparent portions of the micropattern form at least one alphanumeric character.
      58. The adaptive solid-state optical test target of any one of embodiments 46-57, wherein the transparent portions of the micropattern comprise regions of the top surface of the second substrate without the opaque coating.
      59. The adaptive solid-state optical test target of any one of embodiments 46-58, wherein the transparent portions of the micropattern form a plurality of pinholes, wherein the plurality of pinholes fill a full field of view when the solid-state optical test target is positioned in an optical imaging system which comprises at least one objective lens, at least one tube lens, and a camera.
      60. The adaptive solid-state optical test target of any one of embodiments 46-59, wherein the transparent portions of the micropattern form a non-repeating rotationally symmetrical shape including concentric circles.
      61. The adaptive solid-state optical test target of embodiment 60, wherein the symmetrical shape comprises a bullseye or plus sign (+).
      62. The adaptive solid-state optical test target of any one of embodiments 54-61, wherein the dimension of the transparent portions of the micropattern is about 1 micron.
      63. The adaptive solid-state optical test target of any one of embodiments 46-62, wherein the opaque portions of the micropattern comprising repeating shapes arranged in an array.
      64. The adaptive solid-state optical test target of any one of embodiments 46-63, wherein the opaque portions of the micropattern form a plurality of one type of shape, or a mixture of different types of shapes, where the one type of shape and the mixture of different types of shapes are selected from a group consisting of circles, squares and triangles.
      65. The adaptive solid-state optical test target of any one of embodiments 46-64, wherein the opaque portions of the micropattern forms the shape of at least one line.
      66. The adaptive solid-state optical test target of any one of embodiments 46-65, wherein the opaque portions of the micropattern form at least one alphanumeric character.
      67. The adaptive solid-state optical test target of any one of embodiments 46-66, wherein the opaque portions of the micropattern form a plurality of pinholes such that the pinholes fill a full field of view when the solid-state optical test target is positioned in an optical imaging system which comprises at least one objective lens, at least one tube lens, and a camera.
      68. The adaptive solid-state optical test target of any one of embodiments 46-67, wherein the opaque portions of the micropattern form a non-repeating rotationally symmetrical shape including concentric circles.
      69. The adaptive solid-state optical test target of embodiment 68, wherein the symmetrical shape comprises a bullseye or plus sign (+).
      70. The adaptive solid-state optical test target of any one of embodiments 63-69, wherein the dimension of the opaque portions of the micropattern is about 1 micron.
      71. The adaptive solid-state optical test target of any one of embodiments 46-70, wherein the height/thickness of the first region of the first substrate [T-top substrate(1)] is related to the refractive index of the first substrate [n-top substrate(1)], the first designated height of the first channel [T-channel(1)] and the refractive index of the first designated fluid [n-fluid(1)], in an equation

$$[ T - top substrate ( 1 ) ] = ( [ ( T - channel ( 1 ) ] * ( [ ( n - fluid ( 1 ) ] / [ n - top substrate ( 1 ) ] ) ) . \qquad ( Equation 2 )$$

72. The adaptive solid-state optical test target of any one of embodiments 46-71, which is positioned in an optical imaging system which comprises at least one objective lens, at least one tube lens, and a camera.
73. The adaptive solid-state optical test target of embodiment 72, wherein the optical imaging system further comprises at least on light source and at least one filter.
74. The adaptive solid-state optical test target of embodiment 73, wherein the at least on light source comprises a laser or LED excitation light.
75. The adaptive solid-state optical test target of embodiment 73 or 74, wherein the at least one light source is positioned to excite a fluorophore in a sample.
76. An adaptive solid-state optical test target comprising:
a) a substrate comprising a transparent medium with a top surface, a bottom surface and one or more side surfaces, the substrate having at least two regions with different thicknesses, wherein the first region has a first thickness and the second region has a second thickness, and the substrate comprises a refractive index of [n-top substrate(1)]; and
b) the substrate comprises at least one layer of fluorescent dye layered on the bottom surface of the substrate, wherein
   (i) at least a portion of the bottom surface of the substrate comprises an opaque coating that forms a micropattern, the micropattern configured to include opaque portions and transparent portions,
   (ii) the at least one fluorescent dye layer is layered on the opaque coating such that the opaque coating is disposed between the bottom surface of the substrate and the at least one fluorescent dye layer,
   (iii) the adaptive solid-state optical test target lacks a flow cell and lacks a liquid,
   (iv) the thickness of the first region of the substrate is configured to simulate the presence of a first hypothetical flow cell located between the first and second substrates, wherein the first hypothetical flow cell includes a first channel having a top surface and a bottom surface, the first channel containing a first fluid, wherein the first channel has a first thickness of [T-channel(1)] and the first fluid has a refractive index of [n-fluid(1)],
   (v) the thickness of the first region of the substrate is configured to permit imaging of the bottom surface of the first channel of the first hypothetical flow cell, and
   (vi) the thickness of the second region of the substrate is configured to permit imaging of the top surface of the first channel of the first hypothetical flow cell.
      77. The adaptive solid-state optical test target of embodiment 76, wherein the bottom surface is flat.
      78. The adaptive solid-state optical test target of embodiment 76 or 77, wherein the first and second regions are flat.
      79. The adaptive solid-state optical test target of any one of embodiments 76-78, wherein the transparent medium comprises transparent glass.
      80. The adaptive solid-state optical test target of any one of embodiments 76-79, wherein the thickness of the opaque coating that forms the micropattern on the bottom surface of the substrate is about 100 nm.
      81. The adaptive solid-state optical test target of any one of embodiments 76-80, wherein the opaque coating comprises Chromium or aluminum.
      82. The adaptive solid-state optical test target of any one of embodiments 76-81, wherein the transparent portions of the micropattern comprise repeating shapes arranged in an array.
      83. The adaptive solid-state optical test target of any one of embodiments 76-82, wherein the transparent portions of the micropattern form a plurality of one type of shape, or a mixture of different types of shapes, where the one type of shape and the mixture of different types of shapes are selected from a group consisting of circles, squares and triangles.
      84. The adaptive solid-state optical test target of any one of embodiments 76-83, wherein the transparent portions of the micropattern forms the shape of at least one line.
      85. The adaptive solid-state optical test target of any one of embodiments 76-84, wherein the transparent portions of the micropattern form at least one alphanumeric character.
      86. The adaptive solid-state optical test target of any one of embodiments 76-85, wherein the transparent portions of the micropattern comprise regions of the top surface of the second substrate without the opaque coating.
      87. The adaptive solid-state optical test target of any one of embodiments 76-86, wherein the transparent portions of the micropattern form a plurality of pinholes, and wherein the plurality of pinholes fill a full field of view when the solid-state optical test target is positioned in an optical imaging system which comprises at least one objective lens, at least one tube lens, and a camera.
      88. The adaptive solid-state optical test target of any one of embodiments 76-87, wherein the transparent portions of the micropattern form a non-repeating rotationally symmetrical shape including concentric circles.
      89. The adaptive solid-state optical test target of embodiment 88, wherein the symmetrical shape comprises a bullseye or plus sign (+).
      90. The adaptive solid-state optical test target of any one of embodiments 82-89, wherein the dimension of the transparent portions of the micropattern is about 1 micron.
      91. The adaptive solid-state optical test target of any one of embodiments 76-90, wherein the opaque portions of the micropattern comprising repeating shapes arranged in an array.
      92. The adaptive solid-state optical test target of any one of embodiments 76-91, wherein the opaque portions of the micropattern form a plurality of one type of shape, or a mixture of different types of shapes, where the one type of shape and the mixture of different types of shapes are selected from a group consisting of circles, squares and triangles.

93. The adaptive solid-state optical test target of any one of embodiments 76-92, wherein the opaque portions of the micropattern forms the shape of at least one line.

94. The adaptive solid-state optical test target of any one of embodiments 76-93, wherein the opaque portions of the micropattern form at least one alphanumeric character.
95. The adaptive solid-state optical test target of any one of embodiments 76-94, wherein the opaque portions of the micropattern form a plurality of pinholes, and wherein the plurality of pinholes fill a full field of view when the solid-state optical test target is positioned in an optical imaging system which comprises at least one objective lens, at least one tube lens, and a camera.
96. The adaptive solid-state optical test target of any one of embodiments 76-95, wherein the opaque portions of the micropattern form a non-repeating rotationally symmetrical shape including concentric circles.
97. The adaptive solid-state optical test target of embodiment 96, wherein the symmetrical shape comprises a bullseye or plus sign (+).
98. The adaptive solid-state optical test target of any one of embodiments 91-97, wherein the dimension of the opaque portions of the micropattern is about 1 micron.
99. The adaptive solid-state optical test target of any one of embodiments 76-98, wherein the height/thickness of the first region of the substrate [T-top substrate(1)] is related to the refractive index of the substrate [n-top substrate(1)], the first designated height of the first channel [T-channel(1)] and the refractive index of the first designated fluid [n-fluid(1)], in an equation

$$[ T - top substrate ( 1 ) ] = ( [ ( T - channel ( 1 ) ] * ( [ ( n - fluid ( 1 ) ] / [ n - top substrate ( 1 ) ] ) ) . \qquad ( Equation 2 )$$

100. The adaptive solid-state optical test target of any one of embodiments 76-99, further comprising a second layer of fluorescent dye layered on the first layer of fluorescent dye.
101. The adaptive solid-state optical test target of embodiment 100, wherein the first layer of fluorescent dye comprises a fluorescent dye having an excitation spectrum of 520-540 nm which can produce a green band emission of 530-630 nm.
102. The adaptive solid-state optical test target of embodiment 101, wherein the first layer of fluorescent dye comprises a mixture of two fluorescent dyes, wherein the first fluorescent dye comprises an excitation spectrum of 520-540 nm which can produce a green band emission of 530-630 nm, and the second fluorescent dye comprises an excitation spectrum of 630-650 nm which can produce a red band emission of 630-690 nm.
103. The adaptive solid-state optical test target of embodiment 101, wherein the first layer of fluorescent dye comprises a first fluorescent dye having an excitation spectrum of 520-540 nm which can produce a green band emission of 530-630 nm, and the second layer of fluorescent dye comprises a second fluorescent dye having an excitation spectrum of 630-650 nm which can produce a red band emission of 630-690 nm.
104. The adaptive solid-state optical test target of any one of embodiments 76-103, wherein the adaptive solid-state optical test target is positioned in an optical imaging system which comprises at least one objective lens, at least one tube lens, and a camera.
105. The adaptive solid-state optical test target of embodiment 104, wherein the optical imaging system further comprises at least on light source and at least one filter.
106. The adaptive solid-state optical test target of embodiment 105, wherein the at least on light source comprises a laser or LED excitation light.
107. The adaptive solid-state optical test target of embodiment 105 or 106, wherein the at least one light source is positioned to excite a fluorophore in a sample.
108. A fluorescent solid-state optical test target comprising:
a) a substrate comprising a transparent medium comprising a top surface, a bottom surface and one or more side surfaces, wherein at least a portion of the bottom surface of the substrate comprises an opaque coating that forms a micropattern, the micropattern configured to include opaque portions and transparent portions, and the substrate having a refractive index of [n-top substrate(1)]; and
b) at least one layer of fluorescent dyes layered on the bottom surface of the substrate where the fluorescent dye layer is layered on the opaque coating, wherein
   (i) the fluorescent solid-state optical test target lacks a flow cell and lacks a liquid,
   (ii) the thickness of the substrate is configured to simulate the presence of a first hypothetical flow cell located between the first and second substrates, wherein the first hypothetical flow cell includes a first channel having a top surface and bottom surface, and the first channel containing a designated first fluid, wherein the first channel has a first designated thickness of [T-channel(1)] and the first designated fluid has a refractive index of [n-fluid(1)], and
   (iii) the thickness of the first substrate is configured to permit imaging of the bottom surface of the first channel of the first hypothetical flow cell.
      109. The fluorescent solid-state optical test target of embodiment 108, wherein the top surface, bottom surface and one or more side surfaces have an even thickness.
      110. The fluorescent solid-state optical test target of embodiment 108 or 109, wherein the top surface and the bottom surface are flat.
      111. The fluorescent solid-state optical test target of any one of embodiments 108-110, wherein the transparent medium comprises transparent glass.
      112. The fluorescent solid-state optical test target of any one of embodiments 108-111, wherein the thickness of the opaque coating that forms the micropattern on the bottom surface of the substrate is about 100 nm.
      113. The fluorescent solid-state optical test target of any one of embodiments 108-112, wherein the opaque coating comprises chromium or aluminum.
      114. The fluorescent solid-state optical test target of any one of embodiments 108-113, wherein the transparent portions of the micropattern comprise repeating shapes arranged in an array.
      115. The fluorescent solid-state optical test target of any one of embodiments 108-114, wherein the transparent portions of the micropattern form a plurality of one type of shape, or a mixture of different types of shapes, where the one type of shape and the mixture of different types of shapes are selected from a group consisting of circles, squares and triangles.
      116. The fluorescent solid-state optical test target of any one of embodiments 108-115, wherein the transparent portions of the micropattern forms the shape of at least one line.
      117. The fluorescent solid-state optical test target of any one of embodiments 108-116, wherein the transparent portions of the micropattern form at least one alphanumeric character.
      118. The fluorescent solid-state optical test target of any one of embodiments 108-117, wherein the transparent portions of the micropattern comprise regions of the bottom surface of the substrate without the opaque coating.
      119. The fluorescent solid-state optical test target of any one of embodiments 108-117, wherein the transparent portions of the micropattern form a plurality of pinholes such that the pinholes fill a full field of view when the solid-state optical test target is positioned in an optical imaging system which comprises at least one objective lens, at least one tube lens, and a camera.
      120. The fluorescent solid-state optical test target of any one of embodiments 108-118, wherein the transparent portions of the micropattern form a non-repeating rotationally symmetrical shape including concentric circles.
      121. The fluorescent solid-state optical test target of embodiment 120, wherein the symmetrical shape comprises a bullseye or plus sign (+).
      122. The fluorescent solid-state optical test target of any one of embodiments 114-121, wherein the dimension of the transparent portions of the micropattern is about 1 micron.
      123. The fluorescent solid-state optical test target of any one of embodiments 108-122, wherein the opaque portions of the micropattern comprising repeating shapes arranged in an array.
      124. The fluorescent solid-state optical test target of any one of embodiments 108-123, wherein the opaque portions of the micropattern form a plurality of one type of shape, or a mixture of different types of shapes, where the one type of shape and the mixture of different types of shapes are selected from a group consisting of circles, squares and triangles.
      125. The fluorescent solid-state optical test target of any one of embodiments 108-124, wherein the opaque portions of the micropattern forms the shape of at least one line.
      126. The fluorescent solid-state optical test target of any one of embodiments 108-125, wherein the opaque portions of the micropattern form at least one alphanumeric character.
      127. The fluorescent solid-state optical test target of any one of embodiments 108-126, wherein the opaque portions of the micropattern form a plurality of pinholes such that the pinholes fill a full field of view when the solid-state optical test target is positioned in an optical imaging system which comprises at least one objective lens, at least one tube lens, and a camera.
      128. The fluorescent solid-state optical test target of any one of embodiments 123-127, wherein the opaque portions of the micropattern form a non-repeating rotationally symmetrical shape including concentric circles.
      129. The fluorescent solid-state optical test target of embodiment 128, wherein the symmetrical shape comprises a bullseye or plus sign (+).
      130. The fluorescent solid-state optical test target of any one of embodiments 123-129, wherein the dimension of the opaque portions of the micropattern is about 1 micron.
      131. The fluorescent solid-state optical test target of any one of embodiments 108-130, wherein the height/thickness of the first region of the substrate [T-top substrate(1)] is related to the refractive index of the substrate [n-top substrate(1)], the first designated height of the first channel [T-channel(1)] and the refractive index of the first designated fluid [n-fluid(1)], in an equation

$$[ T - top substrate ( 1 ) ] = ( [ ( T - channel ( 1 ) ] * ( [ ( n - fluid ( 1 ) ] / [ n - top substrate ( 1 ) ] ) ) . \qquad ( Equation 2 )$$

132. The fluorescent solid-state optical test target of any one of embodiments 108-131, further comprising a second layer of fluorescent dye layered on the first layer of fluorescent dye.
133. The fluorescent solid-state optical test target of any one of embodiments 108-132, wherein the first layer of fluorescent dyes comprises a fluorescent dye having an excitation spectrum of 520-540 nm which can produce a green band emission of 530-630 nm.
134. The fluorescent solid-state optical test target of any one of embodiments 108-133, wherein the first layer of fluorescent dyes comprises a mixture of two fluorescent dyes, the first fluorescent dye having an excitation spectrum of 520-540 nm which can produce a green band emission of 530-630 nm, and the second fluorescent dye having an excitation spectrum of 630-650 nm which can produce a red band emission of 630-690 nm.
135. The fluorescent solid-state optical test target of any one of embodiments 108-134, wherein the first layer of fluorescent dye comprises a first fluorescent dye having an excitation spectrum of 520-540 nm which can produce a green band emission of 530-630 nm, and the second layer of fluorescent dye comprises a second fluorescent dye having an excitation spectrum of 630-650 nm which can produce a red band emission of 630-690 nm.
136. The fluorescent solid-state optical test target of any one of embodiments 108-135, wherein the fluorescent solid-state optical test target is positioned in an optical imaging system which comprises at least one objective lens, at least one tube lens, and a camera.
137. The fluorescent solid-state optical test target of any one of embodiment 136, wherein the optical imaging system further comprises at least on light source and at least one filter.
138. The fluorescent solid-state optical test target of embodiment 137, wherein the at least on light source comprises a laser or LED excitation light.
139. The fluorescent solid-state optical test target of embodiment 137 or 138, wherein the at least one light source is positioned to excite a fluorophore in a sample.
140. A method for evaluating the performance of an optical imaging system, comprising the steps:
a) positioning the solid-state optical test target of any one of embodiments 1-30 in an optical imaging system comprising at least one objective lens, at least one tube lens, a camera, at least on light source and at least one filter;
b) detecting light transmitted through the first substrate; and
c) evaluating the performance of the optical imaging system based on the light that is transmitted through the first substrate.
   141. A method for evaluating the performance of an optical imaging system, comprising the steps:
a) positioning the adaptive solid-state optical test target of any one of embodiments 31-40 in an optical imaging system comprising at least one objective lens, at least one tube lens, a camera, at least on light source and at least one filter;
b) detecting light transmitted through the third substrate; and
c) evaluating the performance of the optical imaging system based on the light that is transmitted through the third substrate.
   142. A method for evaluating the performance of an optical imaging system, comprising the steps:
a) positioning the solid-state optical test target of any one of embodiments 41-44, in an optical imaging system which comprises at least one objective lens, at least one tube lens, a camera, at least on light source and at least one filter;
b) detecting light transmitted through the substrate; and
c) evaluating the performance of the optical imaging system based on the light that is transmitted through the substrate.
   143. A method for evaluating the performance of an optical imaging system, comprising the steps:
a) positioning the solid-state optical test target of embodiment 45, in an optical imaging system which comprises at least one objective lens, at least one tube lens, a camera, at least on light source and at least one filter;
b) detecting light transmitted through the substrate; and
c) evaluating the performance of the optical imaging system based on the light that is transmitted through the substrate.
   144. A method for evaluating the performance of an optical imaging system, comprising the steps:
a) positioning the adaptive solid-state optical test target of any one of embodiments 46-75 in an optical imaging system comprising at least one objective lens, at least one tube lens, a camera, at least on light source and at least one filter;
b) detecting light transmitted through the first region of the first substrate;
c) detecting light transmitted through the second region of the first substrate; and
d) evaluating the performance of the optical imaging system based on the light that is transmitted through the first and second regions of the first substrate.
   145. A method for evaluating the performance of an optical imaging system, comprising the steps:
a) positioning the adaptive solid-state optical test target of any one of embodiments 76-107 in an optical imaging system comprising at least one objective lens, at least one tube lens, a camera, at least on light source and at least one filter;
b) detecting light transmitted through the first region of the first substrate;
c) detecting light transmitted through the second region of the first substrate; and
d) evaluating the performance of the optical imaging system based on the light that is transmitted through the first and second regions of the first substrate.
   146. A method for evaluating the performance of an optical imaging system, comprising the steps:
a) positioning the fluorescent solid-state optical test target of any one of embodiments 108-139 in an optical imaging system comprising at least one objective lens, at least one tube lens, a camera, at least on light source and at least one filter;
b) detecting light transmitted through the substrate; and
c) evaluating the performance of the optical imaging system based on the light that is transmitted through the substrate.
   147. The method of any one of embodiments 140-146, wherein the at least on light source comprises a laser or LED excitation light.
   148. The method of any one of embodiments 140-147, wherein the at least one light source is positioned to excite a fluorophore in a sample.
   149. The method of any one of embodiments 140-148, wherein evaluating the performance of the optical imaging system comprises any one or more of:
(i) determining the accuracy of the optical alignment;
(ii) determining the autofocus accuracy;
(iii) calibrating the light source;
(iv) calibrating the camera;
(v) determining an image uniformity correction;
(vi) determining distortion levels;
(vii) determining contrast across a field of view;
(viii) determining alignment of the camera (sensor);
(ix) determining the focal distance of the camera (sensor);
(x) determining field flatness;
(xi) determining focus repeatability;
(xii) determining spherical aberration or chromatic aberration;
(xiii) determining alignment of different color channels;
(xiv) determining point spread function measurement; and/or
(xv) determining a modulated transfer function (MTF).
   150. A flow cell device comprising:
a support comprising one or more substrates comprising one or more channels;
an inlet in the one or more substrates; and
an outlet in the one or more substrates,
wherein the one or more channels run from the inlet to the outlet, and
wherein the one or more channels comprise a surface coated with a plurality of fluorescent beads that are immobilized to the surface.
   151. The flow cell device of embodiment 150, wherein the plurality of fluorescent beads are chemically immobilized to the surface.
   152. The flow cell device of embodiment 150 or 151, wherein the surface is passivated.
   153. The flow cell device of any of embodiments 150-152, wherein the surface is passivated with a coating that can immobilize polynucleotides.
   154. The flow cell device of embodiment 153, wherein the polynucleotides comprise surface capture primers, nucleic acid template molecules, or both.
   155. The flow cell device of any of embodiments 150-154, wherein the surface comprises a plurality of polynucleotides captured thereon.
   156. The flow cell device of any of embodiments 150-155, wherein the flow cell device is configured to simultaneous image the polynucleotides and the plurality fluorescent beads in a first sequencing cycle via a first channel using a sequencing system.
   157. The flow cell device of any of embodiments 150-156, wherein the polynucleotides are imaged in a first sequencing cycle via a first channel, and the plurality of fluorescent beads are imaged in a second sequencing cycle via a second channel using a sequencing system.
   158. The flow cell device of any of embodiments 150-157, wherein the first cycle is not a dark cycle and the second cycle is a dark cycle.
   159. The flow cell device of any of embodiments 150-158, wherein the plurality of fluorescent beads comprise one, two, three, four, five or six different types of beads, and wherein each type of bead emits a different color or combination of colors in response to excitement by a laser.
   160. The flow cell device of any of embodiments 159, wherein the flow cell devices comprises an about equal amount of the two, three, four, five, or six different types of beads.
   161. The flow cell device of any of embodiments 150-160, wherein at least a portion of the polynucleotides moves relative to the surface and wherein the plurality fluorescent beads remain immobilized relative to the surface from a first sequencing cycle to a second sequencing cycle in a sequencing run.
   162. The flow cell device of any of embodiments 150-161, wherein at least a portion of the polynucleotides move from first positions in a first flow cell image acquired via a first channel in a first sequencing cycle to second positions in a second flow cell image acquired via a second channel in the first sequencing cycle, and wherein the plurality of fluorescent beads remain immobilized relative to the surface in the first and second flow cell images.
   163. The flow cell device of any of embodiments 150-162, wherein the flow cell device is configured to be used on a sequencing system for calibrating the sequencing system.
   164. The flow cell device of any of embodiments 150-163, wherein the sequencing system comprises one, two, three, four, five, or six color channels, and wherein the flow cell devised is used for calibrating the one, two, three, four, five, or six color channels of the sequencing system.
   165. The flow cell device of any of embodiments 150-164, wherein the flow cell device enables image registration of images of the polynucleotides taken on a sequencing system between different sequencing cycles or color channels of the same sequencing cycle, or a combination thereof, wherein the image registration is based on the relative positions of the plurality of fluorescent beads.
   166. The flow cell device of any of embodiments 150-165, wherein the one or more substrates comprises a top substrate and a bottom substrate.
   167. The flow cell device of embodiment 166, wherein the one or more channels are defined between the top substrate and the bottom substrate.
   168. The flow cell device of any of embodiments 150-167, wherein the surface is an interior top surface, an interior bottom surface, or both, of the one or more channels.
   169. The flow cell device of any of embodiments 150-168, wherein the plurality of fluorescent beads emit a first fluorescent light in response to laser excitement in a first sequencing cycle in a first sequencing run.
   170. The flow cell device of any of embodiments 150-169, wherein the first fluorescent light comprises a first wavelength, a first intensity, a first color, or a combination thereof.
   171. The flow cell device of any of embodiments 150-170, wherein the plurality of fluorescent beads emit a second fluorescent light in response to laser excitement in an additional sequencing cycle in the first sequencing run.
   172. The flow cell device of embodiment 171, wherein the additional sequencing cycle is a 100th cycle, a 110th cycle, a 120th cycle, or a 130th cycle.
   173. The flow cell device of embodiment 171 or 172, wherein the second fluorescent light comprises a second wavelength, a second intensity, a second color, or a combination thereof.
   174. The flow cell device of embodiment 173, wherein the second intensity is less than about 10%, 8%, or 5% different from the first intensity.
   175. The flow cell device of any of embodiments 150-174, wherein the plurality of fluorescent beads emit a third fluorescent light in response to laser excitement in a first sequencing cycle in a second sequencing run after storage of the flow cell device.
   176. The flow cell device of embodiment 175, wherein the storage comprises about 6 months at about room temperature.
   177. The flow cell device of embodiment 175 or 176, wherein the third fluorescent light comprises a third wavelength, a third intensity, a third color, or a combination thereof.
   178. The flow cell device of any of embodiments 175-177, wherein the third intensity is less than about 10%, 8%, or 5% different from the first intensity.
   179. The flow cell device of any of embodiments 150-178, wherein the plurality of fluorescent beads emit a fourth fluorescent light in response to laser excitement in a first sequencing cycle in a third sequencing run after exposing the flow cell device for about 30 minutes to an about 100° C. environment after the first sequencing run.
   180. The flow cell device of embodiment 179, wherein the fourth fluorescent light comprises a fourth wavelength, a fourth intensity, a fourth color, or a combination thereof.
   181. The flow cell device of embodiment 179 or 180, wherein the fourth intensity is about less than 10%, 8%, or 5% different from the first intensity.
   182. The flow cell device of any of embodiments 150-181, wherein the plurality of fluorescent beads emit a fifth fluorescent light in response to laser excitement in a first sequencing cycle in a fourth sequencing run after drying the flow cell device and refilling the flow cell with reagents at least once, twice, 5 times, 10 times, or 15 times after the first sequencing run.
   183. The flow cell device of embodiment 182, wherein the flow cell has been dried and refilled more than 20 times after the first sequencing run.
   184. The flow cell device of embodiment 182 or 183, wherein the fifth fluorescent light comprises a fifth wavelength, a fifth intensity, a fifth color, or a combination thereof.
   185. The flow cell device of any of embodiments 182-184, wherein the fifth intensity is less than about 10%, 8%, or 5% different from the first intensity.
   186. The flow cell device of any of embodiments 150-185, wherein two or more of the first, second, third, fourth, and fifth wavelengths are about identical.
   187. The flow cell device of any of embodiments 150-186, wherein the first fluorescent light is obtained from a first channel, and wherein the plurality of fluorescent beads emit sixth fluorescent light in response to laser excitement in the first sequencing cycle in a second channel in the first sequencing run.
   188. The flow cell device of embodiment 187, wherein the sixth fluorescent light comprises a sixth wavelength, a sixth intensity, a sixth color, or a combination thereof.
   189. The flow cell device of embodiment 187 or 188, wherein the sixth intensity is about less than 10%, 8%, or 5% different from the first intensity.
   190. The flow cell device of any of embodiments 187-189, wherein two or more of the first, second, third, fourth, fifth, and sixth wavelengths are about identical.
   191. The flow cell device of any of embodiments 187-190, wherein the first and the sixth wavelengths are different, and the first and the sixth colors are different.
   192. The flow cell device of any of embodiments 150-191, wherein the first, second, third, fourth, fifth, or sixth wavelength is within a range from about 150 nm to about 850 nm.
   193. The flow cell device of any of embodiments 150-192, wherein the first, second, third, fourth, fifth, or sixth color is red, green, blue, yellow, or a combination thereof.
   194. The flow cell device of any of embodiments 150-193, wherein two or more of the first, second, third, fourth, fifth, and sixth colors are about identical.
   195. The flow cell device of any of embodiments 150-194, wherein the fluorescent beads are about randomly distributed on the surface.
   196. The flow cell device of any of embodiments 150-195, wherein an imaging area on the surface comprises about 150,000 to about 450,000 fluorescent beads.
   197. The flow cell device of any of embodiments 150-196, wherein an imaging area comprises at least a portion of a subtile of the flow cell device.
   198. The flow cell device of any of embodiments 150-197, wherein the fluorescent beads comprise microspheres loaded with fluorescent dyes.
   199. The flow cell device of embodiment 198, wherein the microspheres comprise a diameter of about 0.1 μm to about 1.0 um.
   200. The flow cell device of any of embodiments 150-199, wherein the fluorescent beads comprise quantum dots.
   201. The flow cell device of any of embodiments 150-200, wherein the one or more substrates comprise glass or plastic.
   202. The flow cell device of any of embodiments 150-201, wherein the first, second, or sixth wavelength, the first, second, or sixth color, the first, second, or sixth intensity, or combinations thereof are configured to enable image registration of polynucleotides imaged using a sequencing system between different sequencing cycles or between different color channels.
   203. The flow cell device of any of embodiments 150-202, wherein the first, second, sixth wavelength, the first, second, or sixth color, the first, second, or sixth intensity, or combinations thereof are configured to enable calibration of a sequencing system.
   204. The flow cell device of any of embodiments 150-203, wherein the fluorescent beads are covalently attached to the surface.
   205. The flow cell device of any of embodiments 150-204, wherein at least a portion of the polynucleotides move from first positions in a first flow cell image acquired via a first channel in a first sequencing cycle to second positions in a second flow cell image acquired via a second channel in a second sequencing cycle, wherein fluorescent beads in the plurality remain immobilized relative to the surface in the first and second flow cell images, and wherein the first sequencing cycle and the second sequencing cycle are different.
   206. The flow cell device of any of embodiments 150-205, wherein at least a portion of the polynucleotides move from first positions in a first flow cell image acquired via a first channel in a first sequencing cycle to second positions in a second flow cell image acquired via a second channel in a second sequencing cycle, wherein fluorescent beads in the plurality remain immobilized relative to the surface in the first and second flow cell images, and wherein the first sequencing cycle and the second sequencing cycle are different and wherein the first channel and the second channel are identical.
   207. The flow cell device of any of embodiments 150-206, wherein at least part of the support is transparent.
   208. The flow cell device of any of embodiments 150-207, wherein at least part of the one or more substrates is transparent.
   209. The flow cell device of any of embodiments 150-208, wherein the support is solid.
   210. The flow cell device of any of embodiments 150-209, wherein an intensity of some or all of the fluorescent beads are less than 50%, 40%, 30%, 20%, or 10% different from an intensity of some or all of polynucleotides in flow cell images obtained from a same channel.
   211. The flow cell device of any of embodiments 164-210, wherein the one or more channels comprises 1, 2, 3, 4, 5, 6, 7, or 8 channels.
   212. The flow cell device of any of embodiments 150-211, wherein a first sequencing cycle and a second sequencing cycle are a same sequencing cycle.
   213. The flow cell device of any of embodiments 150-212, wherein a first sequencing cycle and a second sequencing cycle are different sequencing cycles.
   214. The flow cell device of any of embodiments 150-213, wherein the first channel and the second channel are a same channel.
   215. The flow cell device of any of embodiments 150-180, wherein the first channel and the second channel are a different channel.
   216. A method of calibrating a sequencing system, the method comprising:
generating first flow cell images by imaging the flow cell device of any one of embodiments 150-215 using a sequencing system in a first sequencing cycle using a first channel;
generating second flow cell images by imaging the flow cell device using the sequencing system in a second sequencing cycle using a second channel; and
calibrating the sequencing system by analyzing the first flow cell images and the second flow cell images.
   217. The method of embodiment 216, wherein the first and second sequencing cycle are a same cycle.
   218. The method of embodiment 216, wherein the first and second sequencing cycle are different cycles.
   219. The method of embodiment 216, wherein the first and second channel are a same channel.
   220. The method of embodiment 216, wherein the first and second channel are different channels.
   221. A method of performing image registration of flow cell images, the method comprising:
generating first images by imaging the flow cell device of any one of embodiments 150-215 using a sequencing system in a first sequencing cycle using a first channel;
generating second images by imaging the flow cell device using the sequencing system in a second sequencing cycle using a second channel; and
performing image registration by analyzing the first flow cell images and the second flow cell images.
   222. The method of embodiment 221, wherein the first and second sequencing cycle are a same cycle.
   223. The method of embodiment 221, wherein the first and second sequencing cycle are different cycles.
   224. The method of embodiment 221, wherein the first and second channel are a same channel.
   225. The method of embodiment 221, wherein the first and second channel are different channels.
   226. A method of performing image registration of flow cell images, the method comprising:
generating first images by imaging the flow cell device of any one of embodiments 150-215 using a sequencing system in a first sequencing cycle using a first channel;
generating second images by imaging the flow cell device using the sequencing system in a second sequencing cycle using a second channel; and
performing image registration based on positions of fluorescent beads and positions of polynucleotides of the flow cell device in the first flow cell images and the second flow cell images.
   227. The method of embodiment 226, wherein the first and second sequencing cycle are a same cycle.
   228. The method of embodiment 226, wherein the first and second sequencing cycle are different cycles.
   229. The method of embodiment 226, wherein the first and second channel are a same channel.
   230. The method of embodiment 226, wherein the first and second channel are different channels.

It is to be appreciated that the Detailed Description section, and not any other section, is intended to be used to interpret the claims. Other sections may set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries may be defined as long as the specified functions and relationships (or equivalents thereof) are appropriately performed. Also, alternative embodiments may perform functional blocks, steps, operations, methods, etc. using orderings different from those described herein.

References herein to “one embodiment,” “an embodiment,” “an example embodiment,” “some embodiments,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein.

Additionally, some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

While preferred embodiments of the present invention have been shown and described herein, it will be obvious to those skilled in the art that such embodiments are provided by way of example only. The breadth and scope of this disclosure should not be limited by any of the above-described exemplary embodiments. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of using an optical test target for evaluating performance of optical imaging systems, the method comprising:
positioning the optical test target in an optical imaging system, wherein the optical test target comprises a first substrate, a second substrate, and an opaque coating that forms a micropattern, the micropattern including opaque portions and transparent portions;
detecting a light signal transmitted through the optical test target using an image sensor of the optical imaging system;
evaluating performance of the optical imaging system based on detection of the light signal; and
adjusting the optical imaging system based on the evaluation.

2. The method of claim 1 further comprising:
imaging one or more samples during sequencing using the optical imaging system without removing the optical test target from the optical imaging system,
wherein the optical imaging system is comprised in a sequencing system.

3. The method of claim 2, wherein the one or more samples comprises in situ cells or tissue.

4. The method of claim 2, wherein performing sequencing of one or more samples using the adjusted optical imaging system without removing the optical test target from the optical imaging system comprises:
illuminating the one or more samples using an excitation light source of the optical imaging system, wherein the excitation light source is different from an illumination light source of the optical imaging system for generating the light signal for the optical test target.

5. The method of claim 1, wherein positioning the optical test target in the optical imaging system comprises:
positioning the optical test target in the optical imaging system permanently.

6. The method of claim 2, wherein positioning the optical test target in an optical imaging system comprises:
positioning the optical test target in the optical imaging system in a location without interference with performing sequencing of one or more samples using the sequencing system.

7. The method of claim 1, wherein detecting light signal transmitted through the optical test target using the image sensor of the optical imaging system comprises:
detecting light transmitted through the first substrate but not transmitted through the second substrate.

8. The method of claim 1, wherein detecting the light signal transmitted through the optical test target using the image sensor of the optical imaging system comprises:
generating one or more images based on detected light transmitted through the optical test target using a hardware processor of a sequencing system.

9. The method of claim 1, wherein the optical test target comprises:
(a) the first substrate comprising a transparent medium, and having a top surface and a bottom surface, wherein the first substrate has a refractive index of [n-top substrate(1)]; and
(b) the second substrate having top surface and a bottom surface, wherein the micropattern is disposed upon the second substrate, wherein
the first substrate is positioned on top of the second substrate, and the first substrate is positioned in direct contact with the micropattern,
the thickness of the first substrate simulates the presence of a first hypothetical flow cell located between the first and second substrates, wherein the first hypothetical flow cell includes a first channel having a top surface and a bottom surface, the first channel containing a first fluid, wherein the first channel has a first thickness of [T-channel(1)] and the first fluid has a refractive index of [n-fluid(1)], and
the thickness of the first substrate is configured to permit imaging of the bottom surface of the first channel of the first hypothetical flow cell.

10. The method of claim 2, wherein the opaque coating is configured to allow light signal at the first substrate to leak through the opaque coating, and wherein a level of the light signal via leakage is less than ±20% different from a background signal level in an image of the one or more samples acquired using the optical imaging system during a sequencing run.

11. The method of claim 2, wherein the micropattern comprises one or more first features and one or more second features, and wherein the one or more second features comprise transparent portions of the micropattern having a first size in a range from 80% to 120% of a second size of a cluster or polony immobilized on a flow cell device during a sequencing run.

12. The method of claim 1, wherein evaluating performance of the optical imaging system based on detection of the light signal comprises:
determining color cross talk between two or more color channels of the optical imaging system.

13. The method of claim 12, wherein determining color cross talk between two or more color channels of the optical imaging system comprises:
determining an intensity level of the transparent portions in a first image of the optical test target acquired using a first color channel having a predetermined emission filter of the optical imaging system; and
if the intensity level of the transparent portions is greater than a predetermined threshold, determining that color cross talk exists between the two or more color channels,
wherein the predetermined emission filter is configured to only pass a light signal within a wavelength range that is identical to a second wavelength range of emission light of a second color channel of the optical imaging system.

14. The method of claim 1, wherein evaluating performance of the optical imaging system based on detection of the light signal comprises:
determining chromatic aberration of the optical imaging system.

15. The method of claim 14, wherein determining chromatic aberration of the optical imaging system comprises:
determining spatial offsets of the micropattern at two different locations in a first image of the optical test target acquired using a first color channel and in a second image of the optical test target acquired using a second color channel of the optical imaging system.

16. The method of claim 1, wherein evaluating performance of the optical imaging system based on the detected light signal comprises:
determining optical alignment of two or more color channels of the optical imaging system;
determining field flatness of the optical imaging system;
determining level of focus of the transparent portions; or
determining motion of the optical test target caused by a sequencing system.

17. The method of claim 1, wherein the light signal transmitted through the optical test target comprises a first and second color, and wherein a variance in power of the light signal in the first and second color is less than ±10%.

18. The method of claim 17, wherein the first color of the light signal transmitted through the optical test target comprises a variance in power across a field of view (FOV) of the optical test target, and wherein the variance in power across the FOV is less than ±10%.

19. The method of claim 1 further comprising:
directing the light signal from an illumination source to the optical test target via a first light path.

20. The method of claim 19, wherein the first light path is different from an excitation light path from an excitation light source of the optical imaging system to one or more samples to be sequenced using the optical imaging system.

21. The method of claim 1, wherein the optical test target lacks a fluorescent dye or a fluorophore.

22. The method of claim 2, wherein positioning the optical test target in the optical imaging system comprises:
positioning the optical test target at a same z level along a z axis but a different (x,y) location as the one or more samples to be sequenced using the sequencing system, wherein the z axis is orthogonal to a sample plane or sample stage of the sequencing system.

23. The method of claim 1, wherein evaluating performance of the optical imaging system based on detection of the light signal comprises:
evaluating performance of the optical imaging system based on the light signal of one or more first features comprising transparent portions of the micropattern of the optical test target; and
subsequent to adjusting the optical imaging system based on the evaluation, the method further comprises:
detecting second light signal transmitted through the optical test target using the image sensor of the optical imaging system;
evaluating performance of the optical imaging system based on detection of the second light signal of one or more second features comprising transparent portions of the micropattern; and
adjusting the optical imaging system based on the evaluation.

24. A solid-state optical test target comprising:
a) a first substrate comprising a transparent medium, and having a top surface and a bottom surface, wherein the first substrate has a refractive index of [n-top substrate(1)]; and
b) a second substrate having top surface and a bottom surface, wherein
(i) at least a portion of the top surface of the second substrate comprises an opaque coating that forms a micropattern, the micropattern configured to include opaque portions and transparent portions,
(ii) the first substrate is positioned on top of the second substrate, and the first substrate is positioned in direct contact with the micropattern of the second substrate,
(iii) the solid-state optical test target lacks a flow cell and lacks a liquid,
(iv) the thickness of the first substrate simulates the presence of a first hypothetical flow cell located between the first and second substrates, wherein the first hypothetical flow cell includes a first channel having a top surface and a bottom surface, the first channel containing a first fluid, wherein the first channel has a first thickness of [T-channel(1)] and the first fluid has a refractive index of [n-fluid(1)], and
(v) the thickness of the first substrate is configured to permit imaging of the bottom surface of the first channel of the first hypothetical flow cell.

25. A method of sequencing a sample comprising:
(A) positioning a solid-state optical test target in a sequencing system comprising an optical imaging system before sequencing the sample, the solid-state optical test target comprising:
a) a first substrate comprising a transparent medium, and having a top surface and a bottom surface, wherein the first substrate has a refractive index of [n-top substrate(1)]; and
b) a second substrate having top surface and a bottom surface, wherein
(i) at least a portion of the top surface of the second substrate comprises an opaque coating that forms a micropattern, the micropattern configured to include opaque portions and transparent portions, wherein the opaque portions and/or the transparent portions of the micropattern form a plurality of one type of shape or a mixture of different types of shapes, wherein the one type of shape or the mixture of different types of shapes are selected from the group consisting of circles, squares and triangles,
(ii) the first substrate is positioned on top of the second substrate, and the first substrate is positioned in direct contact with the micropattern on the second substrate,
(iii) the solid-state optical test target lacks a flow cell and lacks a liquid,
(iv) the thickness of the first substrate simulates the presence of a first hypothetical flow cell located between the first and second substrates, wherein the first hypothetical flow cell includes a first channel having a top surface and bottom surface, the first channel containing a first fluid, wherein the first channel has a first thickness of [T-channel(1)] and the first fluid has a refractive index of [n-fluid(1)], and
(v) the thickness of the first substrate is configured to permit imaging of the bottom surface of the first channel of the first hypothetical flow cell;
(B) evaluating the performance of the optical imaging system; and
(C) subjecting the sample to next-generation sequencing using the sequencing system.

26. An adaptive solid-state optical test target comprising:
a) a first substrate comprising a transparent medium and a top surface, a bottom surface and one or more side surfaces, the first substrate having at least two regions comprising different thicknesses, wherein the first region and has a first thickness and the second region and has a second thickness, and the first substrate comprises a refractive index of [n-top substrate(1)]; and
b) a second substrate comprising a top surface, a bottom surface and one or more side surfaces, the top surface being flat, wherein
(i) at least a portion of the top surface of the second substrate comprises an opaque coating that forms a micropattern, wherein the micropattern is configured to include opaque portions and transparent portions,
(ii) the first substrate is positioned on top of the second substrate, and the first substrate is positioned in direct contact with the micropattern on the second substrate,
(iii) the solid-state optical test target lacks a flow cell and lacks a liquid,
(iv) the thickness of the first region of the first substrate is configured to simulate the presence of a first hypothetical flow cell located between the first and second substrates, wherein the first hypothetical flow cell includes a first channel having a top surface and a bottom surface, and the first channel comprises a first fluid, wherein the first channel has a first thickness of [T-channel(1)] and the first fluid has a refractive index of [n-fluid(1)],
(v) the thickness of the first region of the first substrate is configured to permit imaging of the bottom surface of the first channel of the first hypothetical cell, and
(vi) the thickness of the second region of the first substrate is configured to permit imaging of the top surface of the first channel of the first hypothetical cell.

27. An adaptive solid-state optical test target comprising:
a) a substrate comprising a transparent medium with a top surface, a bottom surface and one or more side surfaces, the substrate having at least two regions with different thicknesses, wherein the first region has a first thickness and the second region has a second thickness, and the substrate comprises a refractive index of [n-top substrate(1)]; and
b) the substrate comprises at least one layer of fluorescent dye layered on the bottom surface of the substrate, wherein
(i) at least a portion of the bottom surface of the substrate comprises an opaque coating that forms a micropattern, the micropattern configured to include opaque portions and transparent portions,
(ii) the at least one fluorescent dye layer is layered on the opaque coating such that the opaque coating is disposed between the bottom surface of the substrate and the at least one fluorescent dye layer,
(iii) the adaptive solid-state optical test target lacks a flow cell and lacks a liquid,
(iv) the thickness of the first region of the substrate is configured to simulate the presence of a first hypothetical flow cell located between the substrate and a second substrate, wherein the first hypothetical flow cell includes a first channel having a top surface and a bottom surface, the first channel containing a first fluid, wherein the first channel has a first thickness of [T-channel(1)] and the first fluid has a refractive index of [n-fluid(1)],
(v) the thickness of the first region of the substrate is configured to permit imaging of the bottom surface of the first channel of the first hypothetical flow cell, and
(vi) the thickness of the second region of the substrate is configured to permit imaging of the top surface of the first channel of the first hypothetical flow cell.
